(19) United States
(12) Patent Application Publication
(10) Pub. No.: US 2026/0146061 A1
(43) Pub. Date: May 28, 2026

(54) SYSTEMS AND METHODS FOR END TO END CONTINUOUS DOWNSTREAM PROCESSING

(71) Applicant: AstraZeneca AB, Södertälje (SE)

(72) Inventors: Jon COFFMAN, Gaithersburg, MD (US); Sevda DELDARI, Gaithersburg, MD (US); Ujwal PATIL, Gaithersburg, MD (US); Irina RAMOS, Gaithersburg, MD (US); Keith MORGAN, Gaithersburg, MD (US); Taylor BLOOM, Gaithersburg, MD (US); Kareem FAKHFAKH, Gaithersburg, MD (US)

(21) Appl. No.: 18/873,009

(22) PCT Filed: Jun. 9, 2023
(86) PCT No.: PCT/IB2023/055975

§ 371 Date: Dec. 9, 2024

Related U.S. Application Data
(60) Provisional application No. 63/366,159, filed on Jun. 10, 2022.

(51) Int. Cl. C07K 1/34; C07K 1/18

(52) U.S. Cl. CPC C07K 1/34; C07K 1/18

(57) ABSTRACT

Provided herein are methods of purifying proteins of interest using continuous counter-current downstream processing. Also provided herein are methods of purifying proteins of interest using continuous counter-current affinity nanoparticle dialysis.

### CROSS-REFERENCE TO RELATED APPLICATIONS

This application claims priority benefit of U.S. Provisional Patent Application No. 63/366,159, filed Jun. 10, 2022, which is incorporated by reference herein in its entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 50,990 Byte xml file named “CMTFF-100-WO-PCT.xml” created on Jun. 7, 2023.

### BACKGROUND OF THE DISCLOSURE

As biologics move to the forefront of drug development, the need for improved manufacturing processes has grown. With increasing projected demands for recombinant protein therapeutics, more cost effective and flexible manufacturing processes are needed. Indeed, various economic analyses estimate that process development and clinical manufacturing costs can constitute 40-60 percent of a drug's development cost. Along with commercial manufacturing, driven largely by downstream processing of consumable material costs, this can reach up to 25 percent of the sales revenue for a biologic. Accordingly, there is a need for more efficient downstream processing.

### SUMMARY OF THE DISCLOSURE

The present disclosure is directed to a method for purifying a product of interest using counter-current flow filtration, comprising: (a) contacting a first solution comprising the product of interest and impurities with a binding molecule to form a complex, wherein the complex comprises the product of interest bound to the binding molecule; (b) contacting a first flow solution comprising the complex with a first side of a semi-permeable membrane, wherein the complex has a molecular weight that exceeds the molecular weight cutoff of the semi-permeable membrane such that the complex is retained on the first side of the membrane; (c) passing the impurities through the semi-permeable membrane, wherein the impurities have a molecular weight below the molecular weight cutoff of the semi-permeable membrane and are retained on the second side of the semi-permeable membrane in a second flow solution that is counter-current to the first flow solution; and (d) dissociating the complex to form free product of interest and free binding molecule.

In one aspect, the method further comprises (e) regenerating the binding molecule, wherein the regenerated binding molecule is capable of forming a complex upon contact with the product of interest in the first solution or a second solution.

The present disclosure also provides a method for purifying a product of interest using counter-current flow filtration, comprising: (a) contacting a first flow solution comprising the product of interest and impurities with a first side of a semi-permeable membrane, wherein the product of interest passes through the semi-permeable membrane to form a complex with a binding molecule on a second side of a semi-permeable membrane, wherein the complex has a molecular weight that exceeds the molecular weight cutoff of the semi-permeable membrane such that the complex is retained on the second side of the semi-permeable membrane; (b) optionally retaining the impurities on the first side of a semi-permeable membrane or wherein the impurities flow through the semi-permeable membrane, wherein the impurities have a molecular weight below the molecular weight cutoff of the semi-permeable membrane and are either retained on the first side of the semi-permeable membrane in the first flow solution that is counter-current to the second flow solution or pass through the semi-permeable membrane to the second side of the semi-permeable membrane; wherein the second flow solution flow rate is lower than first flow solution flow rate, and (c) dissociating the complex to form free product of interest and free binding molecule. The method further comprises (e) regenerating the binding molecule, wherein the regenerated binding molecule is capable of forming a complex upon contact with the product of interest in the first flow solution or the second flow solution. In some aspects, the regenerated binding molecule is again passed through the second side of the semi-permeable membrane in the second flow solution.

In one aspect, the second flow solution comprises a second binding molecule that can bind to impurities in the first solution and/or second flow solution. In another aspect, unbound binding molecules diffuse through the semi-permeable membrane into the second flow solution.

In some aspects, the product of interest is a protein. In one aspect, the binding molecule comprises Protein A, Protein G, cation exchange resin, or anion exchange resin. In another aspect, the binding molecule comprises Protein A. In one aspect, the first solution comprising the product of interest is obtained from a bioreactor. In one aspect, the second flow solution comprises a positively charged polymer. In another aspect, the positively charged polymer is DEAE dextran.

In one aspect, the impurities comprise low molecular weight species. In another aspect, the positively charged polymer binds low molecular weight species that have diffused through the semi-permeable membrane.

In one aspect, steps (a) through (c) are repeated, and wherein the binding molecule comprises Protein A. In another aspect, steps (a) through (c) are repeated, and wherein the binding molecule comprises a cation-exchange resin. In another aspect, steps (a) through (c) are repeated, and wherein the binding molecule comprises an anion-exchange resin.

In some aspects, the binding molecule comprises an assembled nanoparticle. In some aspects, the assembled nanoparticle is an assembled ferritin nanoparticle comprising 24 fusion protein monomers. In some aspects, each fusion protein monomer comprises i) a self-assembling nanoparticle monomer; ii) a linker; and iii) an immunoglobulin binding domain; In some aspects, the fusion protein monomer comprises an amino acid sequence at least 90%, 95%, 98% or 100% identical to one of SEQ ID NOs: 1-3. In some aspects, the immunoglobulin binding domain is a protein A Z-domain. In some aspects, the protein A Z-domain comprises an amino acid sequence at least 90%, 95%, 98% or 100% identical to one of SEQ ID NOs: 4-7. In some aspects, the linker comprises an amino acid sequence selected from one of SEQ ID NOs: 8-27. In some aspects, the fusion protein monomer that is not complexed and/or assembled diffuses through the semi-permeable membrane into the second flow solution. In some aspects, the fusion protein monomer further comprises a purification tag at one terminus of the fusion protein monomer. In some aspects, the purification tag comprises 6, 8 or 10 repeated histidines. In some aspects, the fusion protein monomer further comprises a protease site between the purification tag and the remainder of the fusion protein monomer. In some aspects, fusion protein monomer comprises a protease site is a HRV-3C protease site. In some aspects, the fusion protein monomer comprises an amino acid sequence at least 90%, 95%, 98% or 100% identical to one of SEQ ID NOs: 28-36. In some aspects, the fusion protein monomer comprises an amino acid sequence at least 90%, 95%, 98% or 100% identical to SEQ ID NO: 29, SEQ ID NO: 30 or SEQ ID NO: 32 or is encoded by a nucleic acid comprising SEQ ID NO 52. In some aspects, the fusion protein monomer comprises 2 to 5 immunoglobulin binding domains. In some aspects, the 2 to 5 immunoglobulin binding domains are protein A Z-domains. In some aspects, the 2 to 5 immunoglobulin binding domains are separated from one another by linkers. In some aspects, the fusion protein monomer is capable of assembling into an assembled nanoparticle comprising 24 fusion protein monomers.

In some aspects, steps (b) and (c) are repeated. In some aspects, steps (b) and (c) are repeated 3 times, 4 times, 5 times, 6 times, 7 times, 8 times, 9 times, 10 times or more than 10 times.

In some aspects, permeate from step (b) and/or step (c) is recycled into the first and/or and upstream flow solution. In some aspects, the permeate from step (b) and/or step (c) is recycled into the first flow solution and/or the second flow solution and/or an upstream solution. In one aspect, the dissociated protein is diafiltrated. In some aspects, the semi-permeable membrane is a dialysis membrane.

In one aspect, the first flow solution and/or second flow solution has a flow rate of about 30 to about 60 mL/minute. In another aspect, the first flow solution and second flow solution have the same flow rate. In another aspect, the first flow solution and second flow solution have different flow rates. In a further aspect, the first flow solution and/or second solution are pulsed. In another aspect, the pulsing enhances mass transfer across the semi-permeable membrane. In another aspect, the pulse volume is less than the volume of a pore of the semi-permeable membrane. In another aspect, the pulse volume is about half of the volume of a pore of the semi-permeable membrane. In another aspect, the pulse volume is greater than the volume of a pore of the semi-permeable membrane.

In one aspect, about 0.1 kg/day, about 0.5 kg/day, about 1 kg/day, about 2 kg/day, about 3 kg/day, about 4 kg/day, about 5 kg/day, about 6 kg/day, about 7 kg/day, about 8 kg/day, about 9 kg/day or about 10 kg/day of product of interest is purified. In another aspect, the product of interest comprises an antibody, an antigen binding fragment, a fusion protein, a naturally occurring protein, a chimeric protein, or any combination thereof. In another aspect, the protein comprises an antibody selected from IgM, IgA, IgE, IgD, and IgG, and wherein optionally about 10 mM to about 1M NaCl, or more preferably about 150 mM NaCl, is added to the first flow solution. In another aspect, the protein comprises an antibody and the antibody is an IgG antibody selected from IgG1, IgG2, IgG3, and IgG4. In another aspect, the antibody is a monoclonal antibody.

In one aspect, the first flow solution is concentrated to about 50 g/L to about 100 g/L of product of interest. In one aspect, the product of interest is a monoclonal antibody. In one aspect, the free product of interest is concentrated to about 50 g/L to about 100 g/L.

In one aspect, wherein the complex has a molecular weight that is about 2 times, about 3 times, about 4 times, about 5 times, about 6 times, about 7 times, about 8 times, about 9 times, or about 10 times the size of the molecular weight cutoff of the semi-permeable membrane. In some aspects, steps (a) through (c) are performed a second time and wherein the second flow solution of the second time is added to the first flow solution of the first time, and wherein the MWCO of the filter used the second time has a MWCO the same size or larger than steps (a)-(c) performed the first time. In some aspects, steps (a) through (c) are performed a second time and wherein the first flow solution of the second time is added to the first flow solution of the first time, and wherein the MWCO of the filter used the second time has a MWCO the same size or larger than steps (a)-(c) performed the first time. In some aspects, steps (a) through (c) are performed a third time and wherein the second flow solution of the third time is added to the first flow solution of the second time, and wherein the MWCO of the filter used the third time has a MWCO the same size or larger than steps (a)-(c) performed the first time or the second time. In some aspects, steps (a) through (c) are performed a third time and wherein the first flow solution of the third time is added to the first flow solution of the second time, and wherein the MWCO of the filter used the third time has a MWCO the same size or larger than steps (a)-(c) performed the first time or the second time. In some aspects, steps (a) through (c) the second time and the third time are performed in series. In some aspects, the first flow solution or the second flow solution of the third time flows into the first flow solution of the second time, and/or wherein the first flow solution or the second flow solution of the second time flows into the first flow solution of the first time.

In one aspect, the present disclosure provides a method of using a solution effluent from the filtrate or dialysate of a continuous downstream step as a wash for an upstream step, wherein the upstream step has a filter with a MWCO the same size or larger than the downstream step that generated the effluent.

In one aspect, the present disclosure provides a method of using a solution effluent from the retentate of a continuous downstream step as a wash for an upstream step, where the upstream step has a filter with a MWCO the same size or smaller than the downstream step that generated the effluent, and the filtrate is directed downstream.

### BRIEF DESCRIPTION OF THE DRAWINGS

FIG. 1 shows a schematic of the continuous counter-current affinity colloidal capture step.

FIG. 2 shows critical flux data for the dewatering stage.

FIGS. 3A and 3B. FIG. 3A shows a schematic of three-stage CM dewatering, and FIG. 3B shows a schematic of a complete continuous downstream process.

FIG. 4 shows the size of the complex forming at different molar ratio of sProA/mAb1.

FIGS. 5A and 5B show schematics of three-stages of dewatering and counter-current washing. FIG. 5C shows an affinity colloid separation with soluble Protein A used to capture antibodies. FIG. 5D shows a soluble Protein A purification cycle using dialysis-based Protein A recycling.

FIG. 6 shows the critical flux data for the washing stage.

FIG. 7 shows the effect of salt concentration on partition coefficient of sProA and mAb during first elution step.

FIG. 8 shows data generated during the first elution of CEX polishing step where the partition coefficient of sProA and mAb1 is measured at pH 3 and different concentration of NaCl.

FIG. 9 shows a schematic diagram of a one-stage system. The feed containing mAb, impurities and resin slurry is pumped into the TFF module using peristaltic pump (P1). The permeate flux is regulated by peristaltic pump (P2).

FIG. 10 shows a schematic diagram of single-pass tangential flow filtration ultrafiltration system. The feed containing mAb, impurities and resin slurry is pumped into the TFF module using peristaltic pump (P1). The permeate flux is regulated by peristaltic pump (P2).

FIG. 11 shows the partitioning coefficient of host cell proteins (HCPs) and Kp impurity at various offered HCP concentrations in 10 g/L mAb pool for the 5% Fractogel TMAE resin.

FIGS. 12A-12B. FIG. 12A shows A preparative SEC chromatogram indicating fractions pooled based on analytical SEC column results. FIG. 12B shows A280 HPLC-SEC traces (TSKgel SuperSW mAb) of fractions from each of the characteristic peaks (A3, B3, and C3). Fractions containing only peaks 1 and 2 on HPLC-SEC were pooled. FIG. 12C shows an SEC chromatogram (isocratic elution phase) for 7.8 mL of 2.2 mg/ml dialyzed ferritin (50 mM tris pH7.4) loaded onto an XK26/70 pre-packed Superose 6 column with flowing 50 mM Tris pH 7.4 mobile phase at 40 cm/h and the corresponding SDS PAGE gel of mock pool samples (L1-4) representing each peak (1-4) showing that all peaks principally contain ferritin (31 kDa band).

FIG. 13 shows the results of the method used to create antibody and nanoparticle samples that do not result in precipitation.

FIGS. 14A-14D show affinity chromatograms for (FIG. 14A) soluble protein A (cycle 0), (FIG. 14B) purified Nanoparticle (cycle 1), (FIG. 14C) nanoparticle flowthrough from cycle 1 (Cycle 1b), (FIG. 14D) neutralized eluate from cycle 1 (Cycle 2). All soluble protein A (sProA) was found in the eluate, based on peak area and not mass balance. Nanoparticle also showed binding, elution, and binding again even after elution.

FIG. 15 shows preparative SEC chromatogram overlays showing isocratic elution for three neutral pH runs: Antibody and nanoparticle together, antibody alone, and nanoparticle alone.

FIG. 16 shows SDS-PAGE results from running the fractions of the neutral pH antibody and nanoparticle mixture on preparative SEC.

FIG. 17 shows preparative SEC chromatogram overlays showing isocratic elution of three separate low pH loads: Antibody and nanoparticle together, antibody alone, and nanoparticle alone

FIG. 18 shows SDS-PAGE results from running the fractions of the low pH antibody and nanoparticle mixture on SEC.

FIG. 19 shows a comparison of antibody and nanoparticle mixture on SEC column at neutral pH and low pH.

FIG. 20 shows SDS PAGE Gels for load, retentate, and filtrate samples at neutral and low pH with ladder. An annotated Image of the ladder is included alongside indications of the heavy chain (HC), light chain (LC), and ferritin monomer (NP384). The dotted box highlights lanes demonstrating the filter based separation.

FIG. 21 shows Sieving coefficients list for each of the separations are shown alongside the volumes for retentate and filtrate after centrifugation at 2000 g for 15 s at RT.

FIG. 22 shows normalized heat plot from isothermal titration calorimetry; 0.5 g/L Nanoparticle was titrated against mAb shows average of 14 binding sites per nanoparticle with an equilibrium dissociation constant of 25 nM.

FIG. 23 shows a schematic of a one-stage system. The feed containing mAb, and CM is pumped into the TFF module using peristaltic pump (P1). The permeate flux is regulated by peristaltic pump (P2) on retentate outlet.

FIG. 24 shows size exclusion chromatographs of the size enrichment feed, 0.5 mg/mL nanoparticle the retentate after 2 diavolumes, and final product of a 300 kDa TFF size enrichment

FIG. 25 shows a schematic for a multistage counter-current single-pass TFF

FIG. 26 shows the effect of pre-charging the membrane with conditioned media on operating TMP

FIG. 27 shows a SDS-PAGE gel for a TFF elution step. Polished mAb 0.5 g/L and 0.05 g/L, wells 1 and 2 respectively; pure nanoparticle 0.23 g/L and 0.02 g/L, wells 3 and 4, respectively; well 5-Bio-Rad Precision Plus unstained protein marker; well 6-TFF Feed; TFF permeate samples well P1 to P5; TFF Retentate samples wells R1 to R5.

FIG. 28 shows a Schematic for Continuous Counter-Current Affinity Nanoparticle Dialysis operation (C3ANDo)

FIGS. 29A and 29B. FIG. 29A shows effective diffusion coefficient of mAb in ˜500 kDa membrane at various feed flux and impact on mAb recovery (FIG. 29B).

FIG. 30 shows a scheme for a C3ANDo capture step.

FIG. 31 shows a scheme for a C3ANDo elution investigation.

FIG. 32 shows an SDS-PAGE gel for a C3ANDo Elution step, comprising a re-circulation phase (40-640 min) along with the single-pass (wells 4-9) samples, and Bio-Rad Precision Plus unstained protein marker as a molecular weight ladder.

FIG. 33 shows SDS-PAGE gel for a C3ANDo Elution step, comprising a re-circulation phase (wells 10-12) and single-pass (wells 4-9) samples, and Bio-Rad Precision Plus unstained protein marker as a molecular weight ladder (well 3).

### DETAILED DESCRIPTION

The present disclosure provides a highly effective approach to remove contaminants during protein purification using counter-current filtration, without the need for chromatography. As such, the present disclosure provides methods for purifying a product of interest that uses approximately 1/10^{th} the amount of water and solutions as chromatographic processes.

### 1. Definitions

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a feed medium,” is understood to represent one or more feed mediums. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation of modification, such as conjugation with a labeling component. Also included in the definition are, for example, polypeptides containing one or more analogs of an amino acid (including, for example, unnatural amino acids, etc.), as well as other modifications known in the art. The term “polypeptide” and “protein” as used herein specifically encompass antibodies and Fc domain-containing polypeptides (e.g., immunoadhesins).

An “anion exchange resin” or “anion exchange polymer” refers to a solid or liquid phase which is positively charged, thus having one or more positively charged ligands attached thereto. Any positively charged ligand attached to the solid phase suitable to form the anionic exchange resin can be used, such as quaternary amino groups.

A “cation exchange resin” or “cation exchange polymer” refers to a solid or liquid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described herein.

As used herein, the term “product of interest” is used in its broadest sense to include any product (either natural or recombinant) including proteins, nucleic acids, macromolecules, or lipids present in a mixture, for which purification is desired. Such product of interest includes, without limitation, enzymes, hormones, growth factors, cyotokines, immunoglobulins (e.g., antibodies), and/or any fusion proteins. Such product of interest may also include DNA, RNA, cDNA, and antisense oligonucleotides. In some aspects, the product of interest refers to any protein that can be produced by the methods described herein. In some aspects, the product of interest refers to any DNA or RNA sequence that can be produced by the methods described herein. In some aspects, the product of interest is a protein of interest.

As used herein, the term “protein of interest” is used in its broadest sense to include any protein (either natural or recombinant), present in a mixture, for which purification is desired. Such proteins of interest include, without limitation, enzymes, hormones, growth factors, cytokines, immunoglobulins (e.g., antibodies), and/or any fusion proteins. In some aspects, the protein of interest refers to any protein that can be produced by the methods described herein. In some aspects, the protein of interest is an antibody. In some aspects, the protein of interest is a recombinant protein.

The terms “purifying,” “separating,” or “isolating,” as used interchangeably herein, refer to increasing the degree of purity of a product of interest from a composition or sample comprising the product of interest and one or more impurities. Typically, the degree of purity of the product of interest is increased by removing (completely or partially) at least one impurity from the composition.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

As used herein, the term “continuous counter-current modular tangential flow filtration” or “CMTFF” refers to the general technique described herein where tangential flow filtration is used in one or more individual implementations, optionally in series or in parallel, wherein the flow of the permeate side of the membrane flows counter-current to the retentate side of the membrane.

As used herein, the term “C3ANDo” or “continuous counter-current affinity nanoparticle dialysis” refers to the general dialysis approach that is used herein, where one or more individual implementations, optionally in series or in parallel, wherein the flow of the lumen side of the dialysis module flows counter-current to the shell side of the module.

As used herein, the term “sProA” or “soluble Protein A” refers to any Protein A that is soluble in solution. Typically, the Protein A is therefore not bound to a chromatography resin or membrane bound. In some aspects, the Protein A is derived from Staphylococcus. The soluble Protein A will retain binding to the Fc region of an IgG immunoglobulin.

As used herein the term “contaminant” is used in its broadest sense to cover any undesired component or compound within a mixture. In cell cultures, cell lysates, or clarified bulk (e.g., clarified cell culture supernatant), contaminants include, for example, host cell nucleic acids (e.g., DNA) and host cell proteins present in a cell culture medium. Host cell contaminant proteins include, without limitation, those naturally or recombinantly produced by the host cell, as well as proteins related to or derived from the product of interest (e.g., proteolytic fragments) and other process related contaminants. In certain embodiments, the contaminant precipitate is separated from the cell culture using another means, such as centrifugation, sterile filtration, depth filtration and tangential flow filtration.

The term “HMW Species” refers to any one or more unwanted proteins present in a mixture. High molecular weight species can include dimers, trimers, tetramers, or other multimers. These species are often considered product related impurities, and can either be covalently or non-covalently linked, and can also, for example, consist of misfolded monomers in which hydrophobic amino acid residues are exposed to a polar solvent, and can cause aggregation.

The term “LMW Species” refers to any one or more unwanted species present in a mixture. Low molecular weight species are often considered product related impurities, and can include clipped species, charge variants, or half molecules for compounds intended to be dimeric (such as monoclonal antibodies).

The term “Host Cell Proteins” or HCP refers to the undesirable proteins generated by a host cell unrelated to the production of the intended product of interest. Undesirable host cell proteins can be secreted into the upstream cell culture supernatant. Undesirable host cell proteins can also be released during cell lysis. The cells used for upstream cell culture require proteins for growth, transcription, and protein synthesis, and these unrelated proteins are undesirable in a final drug product.

The term “fed-batch culture” or “fed-batch culture process” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified.

The term “chromatography” refers to any kind of technique which separates a protein of interest (e.g., an antibody) from other molecules (e.g., contaminants) present in a mixture. Usually, the protein of interest is separated from other molecules (e.g., contaminants) as a result of differences in rates at which the individual molecules of the mixture migrate through a stationary medium under the influence of a moving phase, or in bind and elute processes. The term “matrix” or “chromatography matrix” are used interchangeably herein and refer to any kind of sorbent, resin or solid phase which in a separation process separates a protein of interest (e.g., an Fc region containing protein such as an immunoglobulin) from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g. controlled pore glass), poly(styrenedivinyl)benzene, polyacrylamide, ceramic particles and derivatives of any of the above. Examples for typical matrix types suitable for the method of the present disclosure are cation exchange resins, affinity resins, anion exchange resins or mixed mode resins.

A “ligand” is a functional group that is attached to the chromatography matrix and that determines the binding properties of the matrix. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). Some preferred ligands that can be used herein include, but are not limited to, strong cation exchange groups, such as sulphopropyl, sulfonic acid; strong anion exchange groups, such as trimethylammonium chloride; weak cation exchange groups, such as carboxylic acid; weak anion exchange groups, such as N5N diethylamino or DEAE; hydrophobic interaction groups, such as phenyl, butyl, propyl, hexyl; and affinity groups, such as Protein A, Protein G, and Protein L. In order that the present disclosure can be more readily understood, certain terms are first defined. As used in this application, except as otherwise expressly provided herein, each of the following terms shall have the meaning set forth below. Additional definitions are set forth throughout the application.

The term “affinity chromatography” refers to a protein separation technique in which a protein of interest (e.g., an Fc region containing protein of interest or antibody) is specifically bound to a ligand which is specific for the protein of interest. Such a ligand is generally referred to as a biospecific ligand. In some aspects, the biospecific ligand (e.g., Protein A or a functional variant thereof) is covalently attached to a chromatography matrix material and is accessible to the protein of interest in solution as the solution contacts the chromatography matrix. The protein of interest generally retains its specific binding affinity for the biospecific ligand during the chromatographic steps, while other solutes and/or proteins in the mixture do not bind appreciably or specifically to the ligand. Binding of the protein of interest to the immobilized ligand allows contaminating proteins or protein impurities to be passed through the chromatography matrix while the protein of interest remains specifically bound to the immobilized ligand on the solid phase material. The specifically bound protein of interest is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column. Any component can be used as a ligand for purifying its respective specific binding protein, e.g., antibody. However, in various methods according to the present disclosure, Protein A is used as a ligand for an Fc region containing a target protein. The conditions for elution from the biospecific ligand (e.g., Protein A) of the target protein (e.g., an Fc region containing protein) can be readily determined by one of ordinary skill in the art. In some aspects, Protein G or Protein L or a functional variant thereof can be used as a biospecific ligand. In some aspects, a biospecific ligand such as Protein A is used at a pH range of 5-9 for binding to an Fc region containing protein, washing or re-equilibrating the biospecific ligand/target protein conjugate, followed by elution with a buffer having pH above or below 4 which contains at least one salt. In some aspects, the protein A is not bound to a chromatography column resin and is therefore soluble Protein A or “sProA”.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

The term “conductivity” as used herein, refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is milli Siemens per centimeter (mS/cm), and can be measured using a conductivity meter.

The term “mobile phase” as used herein refers to the liquid or gas that flows through a chromatography system, moving the materials to be separated at different rates over the stationary phase. A mobile phase can be polar or non-polar. Polar mobile phases are commonly employed in connection with non-polar stationary phase, and these chromatography separations are known as reversed phase chromatography. Conversely, non-polar mobile phases are often employed in connection with polar stationary phases, and are commonly known as normal phase chromatography.

The term “stationary phase” as used herein in the context of chromatography refers to the solid or liquid phase of a chromatography system on which the materials to be separated are selectively adsorbed. Commonly, a silica is used as a stationary phase.

The term “chromatography column” or “column” in connection with chromatography as used herein, refers to a container, frequently in the form of a cylinder or a hollow pillar which is filled with the chromatography matrix or resin. The chromatography matrix or resin is the material which provides the physical and/or chemical properties that are employed for purification.

The terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange (CEX), anion exchange (AEX), and mixed mode chromatography.

As used herein “perfusion” or “perfusion culture” or “perfusion culture process” refers to continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This continuous culture strategy generally comprises culturing mammalian cells, e.g., non-anchorage dependent cells, expressing a polypeptide and/or virus of interest during a production phase in a continuous cell culture system.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. In some aspects, an antibody is a full-length antibody.

An immunoglobulin may derive from any of the commonly known isotypes, including but not limited to IgA, secretory IgA, IgG, IgD, IgE, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. The term “antibody” can include multivalent antibodies capable of binding more than two antigens (e.g., trivalent antibody). A trivalent antibody are IgG-shaped bispecific antibodies composed of two regular Fab arms fused via flexible linker peptides to one asymmetric third Fab-sized binding module. This third module replaces the IgG Fc region and is composed of the variable region of the heavy chain fused to CH3 with “knob”-mutations, and the variable region of the light chain fused to CH3 with matching “holes”. The hinge region does not contain disulfide bonds to facilitate antigen access to the third binding site. Where not expressly stated, and unless the context indicates otherwise, the term “antibody” includes monospecific, bispecific, or multi-specific antibodies, as well as a single chain antibody.

The term “antigen-binding portion” or “antigen-binding fragment” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

An “isolated antibody” refers to an antibody that is substantially free of other antibodies having different antigenic specificities (e.g., an isolated antibody that binds specifically to PD-L1 is substantially free of antibodies that bind specifically to antigens other than PD-L1). An isolated antibody that binds specifically to PD-1 may, however, have cross-reactivity to other antigens, such as PD-L1 molecules from different species. Moreover, an isolated antibody can be substantially free of other cellular material and/or chemicals.

A “bispecific” or “bifunctional antibody” is an artificial hybrid antibody having two different heavy/light chain pairs, giving rise to two antigen binding sites with specificity for different antigens. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic, or other techniques known to those skilled in the art.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

In aspects of the fusion protein monomers, the self-assembling nanoparticle monomer is a ferritin monomer. Ferritin is an intracellular protein that stores iron and releases it as needed. Ferritin is widely conserved and is found in almost all living organisms. In its native state, ferritin is a globular protein of 24 subunits that self-assembles to form a hollow nanoparticle. Vertebrates have two types of ferritin, a light subunit (L) with a molecular mass of approximately 19 kDa and a heavy subunit (H) with molecular mass of approximately 21 kDa. Amphibians also express an additional type of ferritin designated as M ferritin. Most organisms express a ferritin that is similar to vertebrate H-type ferritin.

Ferritin is a hollow globular protein of mass 474 kDa and comprises 24 ferritin monomer subunits. Thus, as used herein, An “assembled ferritin nanoparticle” refers to the assembled protein comprising 24 ferritin monomer subunits. In some aspects, the ferritin monomer subunit is a “fusion protein monomer” that contains at least one self-assembling (sa) ferritin monomer sequence, and one or more affinity ligands that are separated by any number of homogenous or heterogeneous linker sequences. A histidine tag (e.g. 8 histidines) can also be included to ease purification by immobilized metal affinity chromatography (IMAC). When a histidine tag is included, cleavable sequence can be included to optionally cleave the histidine tag enzymatically with protease after expression.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “culturing” refers to growing one or more cells in vitro under defined or controlled conditions. Examples of culturing conditions which can be defined include temperature, gas mixture, time, and medium formulation.

The term “inoculation” as used herein refers to the addition of cells to culture medium to start the culture.

The term “induction” or “induction phase” or “growth phase” of the cell culture as used herein refers to the initial seeding of the bioreactor (e.g., seed bioreactor) at the outset of upstream cell culture, and includes the period of exponential cell growth (for example, the log phase) where cells are primarily dividing rapidly. During this phase, the rate of increase in the density of viable cells is higher than at any other time point.

As used herein, the term “production phase” of the cell culture refers to the period of time during which cell growth is stationary or is maintained at a near constant level. The density of viable cells remains approximately constant over a given period of time. Logarithmic cell growth has terminated and protein production is the primary activity during the production phase. The medium at this time is generally supplemented to support continued protein production and to achieve the desired glycoprotein product.

As used herein, the terms “expression” or “expresses” are used to refer to transcription and translation occurring within a cell. The level of expression of a product gene in a host cell can be determined on the basis of either the amount of corresponding mRNA that is present in the cell or the amount of the protein encoded by the product gene that is produced by the cell, or both.

As used herein, the terms “culture medium” and “cell culture medium” and “feed medium” and “fermentation medium” refer to a nutrient solutions used for growing and or maintaining cells, especially mammalian cells. Without limitation, these solutions ordinarily provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range. The nutrient solution can be supplemented electively with one or more components from any of the following categories: (1) hormones and other growth factors such as, serum, insulin, transferrin, and epidermal growth factor; (2) salts, for example, magnesium, calcium, and phosphate; (3) buffers, such as HEPES; (4) nucleosides and bases such as, adenosine, thymidine, and hypoxanthine; (5) protein and tissue hydrolysates, for example peptone or peptone mixtures which can be obtained from purified gelatin, plant material, or animal byproducts; (6) antibiotics, such as gentamycin; (7) cell protective agents, for example pluronic polyol; and (8) galactose. Commercially available media such as Ham's F10 (Sigma), Minimal Essential Medium ((MEM), (Sigma)), RPMI-1640 (Sigma), and Dulbecco's Modified Eagle's Medium ((DMEM), (Sigma)) are suitable for culturing the host cells. In addition, any of the media described in Ham et al., Meth. Enz. 58:44 (1979), Barnes et al., Anal. Biochem. 102:255 (1980) can be used as culture media for the host cells. Any other necessary supplements can also be included at appropriate concentrations.

Various aspects of the disclosure are described in further detail in the following subsections.

### II. Counter-Current Filtration

The present disclosure provides a highly effective approach to remove contaminants during protein purification using counter-current filtration, without the need for chromatography. As such, the present disclosure provides methods for purifying a product of interest that uses approximately 1/10^{th} the amount of water and solutions as chromatographic processes.

The present disclosure provides a method for purifying a product of interest using counter-current flow filtration, comprising: (a) contacting a first solution comprising the product of interest and impurities with a binding molecule to form a complex, wherein the complex comprises the product of interest bound to the binding molecule; (b) contacting a first flow solution comprising the complex with a first side of a semi-permeable membrane, wherein the complex has a molecular weight that exceeds the molecular weight cutoff of the semi-permeable membrane such that the complex is retained on the first side of the membrane; (c) passing the impurities through the semi-permeable membrane, wherein the impurities have a molecular weight below the molecular weight cutoff of the semi-permeable membrane and are retained on the second side of the semi-permeable membrane in a second flow solution that is counter-current to the first flow solution; and (d) dissociating the complex to form free product of interest and free binding molecule, and (e) passing the either of the product of interest or the binding molecule through the semi-permeable membrane, wherein the smaller of either the product of interest or the binding molecule have a molecular weight below the molecular weight cutoff of the semi-permeable membrane and are retained on the second side of the semi-permeable membrane in a second flow solution that is counter-current to the first flow solution, while the larger of either product of interest or the binding molecule is retained on the first side of the membrane.

The present disclosure provides a method for purifying a product of interest using modular counter-current flow filtration, comprising: (a) contacting a first solution comprising the product of interest and impurities with a binding molecule to form a complex, wherein the complex comprises the product of interest bound to the binding molecule; (b) contacting a first flow solution comprising the complex with a first side of a semi-permeable membrane under pressure, wherein the complex has a molecular weight that exceeds the molecular weight cutoff of the semi-permeable membrane such that the complex is retained on the first side of the membrane; (c) passing the impurities through the semi-permeable membrane by convection, wherein the impurities have a molecular weight below the molecular weight cutoff of the semi-permeable membrane to become the filtrate; and (d) dissociating the complex to form free product of interest and free binding molecule, and (e) passing the either the product of interest or the binding molecule through the semi-permeable membrane, wherein the smaller of either the product of interest or the binding molecule have a molecular weight below the molecular weight cutoff of the semi-permeable membrane and are retained on the second side of the semi-permeable membrane in a second flow solution that is counter-current to the first flow solution, while the larger of either product of interest or the binding molecule is retained on the first side of the membrane.

In some cases, the filtrate from one module is used to dilute another solution upstream of the semi-permeable membrane, such that the filtrate is used in a counter current manner.

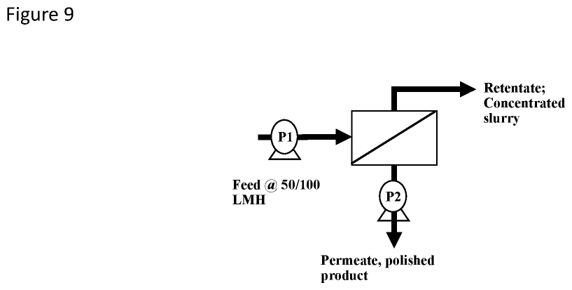

In some aspects, the method further comprises: (e) regenerating the binding molecule by contacting the solution comprising the binding molecule with a first side of a semi-permeable membrane, wherein the binding molecule has a molecular weight that exceeds the molecular weight cutoff of the semi-permeable membrane such that the binding molecule is retained on the first side of the membrane, wherein the regenerated binding molecule is capable of forming a complex upon contact with the product of interest in the first solution or a second solution. See FIG. 9. In some aspects, the second flow solution comprises a second binding molecule that can bind to impurities in the first solution and/or second flow solution. In some aspects, unbound binding molecules diffuse through the semi-permeable membrane into the second flow solution.

In some aspects, the binding molecule comprises Protein A, Proteins G, cation exchange resin, or anion exchange resin. In some aspects, the binding molecule comprises Protein A.

A “cation exchange resin” or “cation exchange membrane” refers to a solid phase which is negatively charged, and which has free anions for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group In some aspects, the cation exchange resin is selected from those having a sulfonate based group (e.g., MonoS, MiniS, Source 15S and 30S, SP SEPHAROSE® Fast Flow, SP SEPHAROSE® High Performance, Capto S, Capto SP ImpRes from GE Healthcare, TOYOPEARL® SP-650S and SP-650M from Tosoh, MACRO-PREP® High S from BioRad, Ceramic HyperD S, TRISACRYL® M and LS SP and Spherodex LS SP from Pall Technologies); a sulfoethyl based group (e.g., FRACTOGEL® SE, from EMD, POROS® S-10 and S-20 from Applied Biosystems); a sulphopropyl based group (e.g., TSK Gel SP 5 PW and SP-5 PW-HR from Tosoh, POROS® HS-20, HS 50, and POROS® XS from Life Technologies); a sulfoisobutyl based group (e.g., FRACTOGEL® EMD SO₃⁻ from EMD); a sulfoxyethyl based group (e.g., SE52, SE53 and Express-Ion S from Whatman), a carboxymethyl based group (e.g., CM SEPHAROSER Fast Flow from GE Healthcare, Hydrocell CM from Biochrom Labs Inc., MACRO-PREP® CM from BioRad, Ceramic HyperD CM, TRISACRYL® M CM, TRISACRYL® LS CM, from Pall Technologies, Matrx CELLUFINE® C500 and C200 from Millipore, CM52, CM32, CM23 and Express-Ion C from Whatman, TOYOPEARL® CM-650S, CM-650M and CM-650C from Tosoh); sulfonic and carboxylic acid based groups (e.g., BAKERBOND® Carboxy-Sulfon from J. T. Baker); a carboxylic acid based group (e.g., WP CBX from J. T Baker, DOWEX®. MAC-3 from Dow Liquid Separations, AMBERLITE® Weak Cation Exchangers, DOWEX® Weak Cation Exchanger, and DIAION® Weak Cation Exchangers from Sigma-Aldrich and FRACTOGEL® EMD COO—from EMD); a sulfonic acid based group (e.g., Hydrocell SP from Biochrom Labs Inc., DOWEX® Fine Mesh Strong Acid Cation Resin from Dow Liquid Separations, UNOsphere S, WP Sulfonic from J. T. Baker, SARTOBIND® S membrane from Sartorius, AMBERLITE® Strong Cation Exchangers, DOWEX® Strong Cation and DIAION® Strong Cation Exchanger from Sigma-Aldrich); or a orthophosphate based group (e.g., P11 from Whatman). Other cation exchange resins include carboxy-methyl-cellulose, BAKERBOND ABX™, Ceramic HyperD Z, Matrex Cellufine C500, Matrex Cellufine C200, or any combination thereof.

An “anion exchange resin” or “anion exchange membrane” refers to a solid phase which is positively charged, thus having one or more positively charged ligands attached thereto. Any positively charged ligand attached to the solid phase suitable to form the anionic exchange resin can be used, such as quaternary amino groups. Commercially available anion exchange resins include DEAE cellulose. In some aspects, the anion exchange resin is selected from DEAE cellulose, POROS® PI 20, PI 50, HQ 10, HQ 20, HQ 50, D 50 from Applied Biosystems, SARTOBIND® Q from Sartorius, MonoQ, MiniQ, Source 15Q and 30Q, Q, DEAE and ANX SEPHAROSER Fast Flow, Q SEPHAROSER High Performance, QAE SEPHADEX® and FAST Q SEPHAROSE® (GE Healthcare), WP PEI, WP DEAM, WP QUAT from J. T. Baker, Hydrocell DEAE and Hydrocell QA from Biochrom Labs Inc., UNOsphere Q, MACRO-PREPR. DEAE and MACRO-PREP® High Q from Biorad, Ceramic HyperD Q, ceramic HyperD DEAE, TRISACRYL® M and LS DEAE, Spherodex LS DEAE, QMA SPHEROSIL® LS, QMA SPHEROSIL® M and MUSTANG® Q from Pall Technologies, DOWEX® Fine Mesh Strong Base Type I and Type II Anion Resins and DOWEX® MONOSPHER E 77, weak base anion from Dow Liquid Separations, INTERCEPT® Q membrane, Matrex CELLUFINE® A200, A500, Q500, and Q800, from Millipore, FRACTOGEL® EMD TMAE, FRACTOGEL® EMD DEAE and FRACTOGEL® EMD DMAE from EMD, AMBERLITE® weak strong anion exchangers type I and II, DOWEX® weak and strong anion exchangers type I and II, DIAION® weak and strong anion exchangers type I and II, DUOLITE® from Sigma-Aldrich, TSK gel Q and DEAE 5 PW and 5 PW-HR, TOYOPEARL® SuperQ-650S, 650M and 650C, QAE-550C and 650S, DEAE-650M and 650C from Tosoh, QA52, DE23, DE32, DE51, DE52, DE53, Express-Ion D or Express-Ion Q from Whatman, and SARTOBIND® Q (Sartorius Corporation, New York, USA). Other anion exchange resins include POROS XQ, Sartobind® Q, Q Sepharose™ XL, Q Sepharose™ big beads, DEAE Sephadex A-25, DEAE Sephadex A-50, QAE Sephadex A-25, QAE Sephadex A-50, Q Sepharose™ high performance, Q Sepharose™ XL, Resource Q, Capto Q, Capto DEAE, Toyopearl GigaCap Q, Fractogel EMD TMAE HiCap, Nuvia Q, or PORGS PI, or any combination thereof.

In some aspects, the first solution comprising the product of interest can be obtained from a bioreactor. In some aspects, the first solution can be obtained from the bioreactor without any other downstream processing steps prior to performing the methods described herein.

In some aspects, the second flow solution comprises a positively charged polymer. In some aspects, the positively charged polymer can comprise DEAE dextran. In some aspects, the positively charged polymer can bind low molecular weight species that have diffused through the semi-permeable membrane.

In some aspects, steps (a) through (c) of the methods described herein are repeated, and wherein the binding molecule comprises Protein A. In some aspects, steps (a) through (c) of the methods described herein are repeated, and wherein the binding molecule comprises cation-exchange resin. In some aspects, steps (a) through (c) of the methods described herein are repeated, and wherein the binding molecule comprises anion-exchange resin.

In some aspects, dissociated protein is diafiltrated.

In some aspects, the first flow solution and/or second flow solution can have a flow rate of at least about 10 mL/minute to about 1,000 mL/minute. In some aspects, the first flow solution and/or second flow solution can have a flow rate of about 10 mL/minute to about 9,000 mL/minute. In some aspects, the first flow solution and/or second flow solution can have a flow rate of about 10 mL/minute to about 8,000 mL/minute. In some aspects, the first flow solution and/or second flow solution can have a flow rate of about 10 mL/minute to about 7,000 mL/minute. In some aspects, the first flow solution and/or second flow solution can have a flow rate of about 10 mL/minute to about 6,000 mL/minute. In some aspects, the first flow solution and/or second flow solution can have a flow rate of about 10 mL/minute to about 5,000 mL/minute. In some aspects, the first flow solution and/or second flow solution can have a flow rate of about 10 mL/minute to about 4,000 mL/minute. In some aspects, the first flow solution and/or second flow solution can have a flow rate of about 10 mL/minute to about 3,000 mL/minute. In some aspects, the first flow solution and/or second flow solution can have a flow rate of about 10 mL/minute to about 2,000 mL/minute. In some aspects, the first flow solution and/or second flow solution can have a flow rate of about 30 mL/minute to about 60 mL/minute, about 31 mL/minute to about 60 mL/min, about 32 mL/minute to about 60 mL/min, about 33 mL/minute to about 60 mL/min, about 34 mL/minute to about 60 mL/min, about 35 mL/minute to about 60 mL/min, about 36 mL/minute to about 60 mL/min, about 37 mL/minute to about 60 mL/min, about 38 mL/minute to about 60 mL/min, about 39 mL/minute to about 60 mL/min, about 40 mL/minute to about 60 mL/min, about 41 mL/minute to about 60 mL/min, about 42 mL/minute to about 60 mL/min, about 43 mL/minute to about 60 mL/min, about 44 mL/minute to about 60 mL/min, about 45 mL/minute to about 60 mL/min, about 46 mL/minute to about 60 mL/min, about 47 mL/minute to about 60 mL/min, about 48 mL/minute to about 60 mL/min, about 49 mL/minute to about 60 mL/min, about 50 mL/minute to about 60 mL/min, about 51 mL/minute to about 60 mL/min, about 52 mL/minute to about 60 mL/min, about 53 mL/minute to about 60 mL/min, about 54 mL/minute to about 60 mL/min, about 55 mL/minute to about 60 mL/min, about 56 mL/minute to about 60 mL/min, about 57 mL/minute to about 60 mL/min, about 58 mL/minute to about 60 mL/min, or about 59 mL/minute to about 60 mL/min. In some aspects, the first flow solution and the second flow solution have the same flow rate. In some aspects, the first and second flow solution have different flow rates.

In some aspects, the first and/or second flow rate can comprise about 30 mL/minute, about 31 mL/minute, about 32 mL/minute, about 33 mL/minute, about 34 mL/minute, about 35 mL/minute, about 36 mL/minute, about 37 mL/minute, about 38 mL/minute, about 39 mL/minute, about 40 mL/minute, about 41 mL/minute, about 42 mL/minute, about 43 mL/minute, about 44 mL/minute, about 45 mL/minute, about 46 mL/minute, about 47 mL/minute, about 48 mL/minute, about 49 mL/minute, about 50 mL/minute, about 51 mL/minute, about 52 mL/minute, about 53 mL/minute, about 54 mL/minute, about 55 mL/minute, about 56 mL/minute, about 57 mL/minute, about 58 mL/minute, about 59 mL/minute, or about 60 mL/minute.

In some aspects, the first flow rate is higher than the second flow rate. In some aspects, the second flow rate is higher than the first flow rate.

In some aspects, the first flow solution and/or the second flow solution are pulsed. In some aspects, the pulsing enhances mass transfer across the semi-permeable membrane. In some aspects, the pulse volume is less than the sum total volume of pores of the semi-permeable membrane. In some aspects, the pulse volume is about half of the sum total volume of pores of the semi-permeable membrane. In some aspects, the pulse volume is greater than the sum total volume of pores of the semi-permeable membrane. In some aspects, a peristaltic pump can be used to generate the pulse in the first and/or second flow solution. In some aspects, a piston pump can be used to generate the pulse in the first and/or second flow solution. In some aspects, a piezoelectric actuator can be used to generate the pulse in the first and/or second flow solution. In some aspects, the pulse flow is not fully reversible, creating a diffusive mass transfer.

In some aspects, the semi-permeable membrane has a molecular weight cutoff of about 1,000 kDa. In some aspects, Protein A has a molecular weight in excess of 1,000 kDa, wherein the Protein A is unable to diffuse through the semi-permeable membrane. In some aspects, the product of interest is a monoclonal antibody with a molecular weight of about 150 kDa. In some aspects, the complex comprising the monoclonal antibody and Protein has a molecular weight exceeding 1,000 kDa. In some aspects, the semi-permeable membrane is a ceramic membrane.

In some aspects, the semi-permeable membrane has a molecular weight cutoff of about 500 kDa to about 1000 kDa. In some aspects, the complex comprising the monoclonal antibody and Protein A has a molecular weight exceeding 1,000 kDa. In some aspects, the Host Cell Proteins (HCP) has a molecular weight of less than 150 kDa.

In some aspects, the complex comprising the product of interest (e.g., monoclonal antibody) and the binding molecule (e.g., Protein A) are dissociated forming free product of interest (e.g., monoclonal antibody) and free binding molecule (e.g., Protein A). In some aspects, the product of interest (e.g., monoclonal antibody) has a molecular weight in excess of the molecular weight cutoff of the semi-permeable membrane and the free binding molecule (e.g., Protein A) has a molecular weight smaller than the molecular weight cutoff of the semi-permeable membrane, wherein the free binding molecule (e.g., Protein A) is removed by diffusion through the semi-permeable membrane. In some aspects, the product of interest (e.g., monoclonal antibody) has a molecular weight smaller than the molecular weight cutoff of the semi-permeable membrane and the free binding molecule (e.g., Protein A) has a molecular weight in excess of the molecular weight cutoff of the semi-permeable membrane, wherein the product of interest (e.g., monoclonal antibody) is removed by diffusion through the semi-permeable membrane.

In some aspects, the molecular weight cutoff of the semi-permeable membrane is from about 1 kDa to about 10 kDa. In some aspects, the molecular weight cutoff of the semi-permeable membrane is from about 100 kDa to 200 kDa. In some aspects, the molecular weight cutoff of the semi-permeable membrane is from about 300 kDa to 400 kDa. In some aspects, the molecular weight cutoff of the semi-permeable membrane is from about 400 kDa to 500 kDa. In some aspects, the molecular weight cutoff of the semi-permeable membrane is from about 500 kDa to 600 kDa. In some aspects, the molecular weight cutoff of the semi-permeable membrane is from about 600 kDa to 700 kDa. In some aspects, the molecular weight cutoff of the semi-permeable membrane is from about 700 kDa to 800 kDa. In some aspects, the molecular weight cutoff of the semi-permeable membrane is from about 900 kDa to 1,000 kDa.

In some aspects, the binding molecule can comprise a Protein A mimetic peptide. In some aspects, the Protein A mimetic peptide can comprise proteinogenic amino acids and/or unnatural amino acids and/or synthetic amino acids arranged non-circular form with no disulfide linkage or in branched form or in circular form with one or more covalent linkage such as disulfide linkage.

In some aspects, the methods described herein are capable of purifying about 0.1 kg/day, about 0.5 kg/day, about 1 kg/day, about 2 kg/day, about 3 kg/day, about 4 kg/day, about 5 kg/day, about 6 kg/day, about 7 kg/day, about 8 kg/day, about 9 kg/day or about 10 kg/day of product of interest.

In some aspects, the purified product of interest is free from residual Protein A.

In some aspects, the methods described herein can comprise a first counter-current filtration, wherein the binding molecule comprises Protein A. In some aspects, the method further comprises a second counter-current filtration, wherein the binding molecule comprises a cation exchange resin or an anion exchange resin. In some aspects, the method further comprises a third counter-current filtration, wherein the binding molecule comprises a cation exchange resin or an anion exchange resin. In some aspects, the ion exchange resin is different between the second and third counter-current filtration steps. In some aspects, the method further comprises a diafiltration step.

In some aspects, the methods described herein are capable of purifying about 0.1 kg/day to about 0.5 kg/day of product of interest. In some aspects, the methods described herein are capable of purifying about 0.5 kg/day to about 1 kg/day of product of interest. In some aspects, the methods described herein are capable of purifying about 1 kg/day to about 2 kg/day of product of interest. In some aspects, the methods described herein are capable of purifying about 2 kg/day to about 3 kg/day of product of interest. In some aspects, the methods described herein are capable of purifying about 3 kg/day to about 4 kg/day of product of interest. In some aspects, the methods described herein are capable of purifying about 4 kg/day to about 5 kg/day of product of interest. In some aspects, the methods described herein are capable of purifying about 5 kg/day to about 6 kg/day of product of interest. In some aspects, the methods described herein are capable of purifying about 6 kg/day to about 7 kg/day of product of interest. In some aspects, the methods described herein are capable of purifying about 7 kg/day to about 8 kg/day of product of interest. In some aspects, the methods described herein are capable of purifying about 8 kg/day to about 9 kg/day of product of interest. In some aspects, the methods described herein are capable of purifying about 9 kg/day to about 10 kg/day of product of interest.

Without wishing to be bound by theory, in some aspects, the methods described herein can use an affinity colloid separation. In some aspects, the methods described herein can use an anion exchange polymer affinity colloid separation. In some aspects, the methods described herein can use Protein A to capture product of interest (e.g., monoclonal antibody) by diffusion through the semi-permeable membrane. In some aspects, the methods described herein can use Protein A to capture product of interest (e.g., monoclonal antibody) and impurities (e.g., HCP) can diffuse through the semi-permeable membrane. In some aspects, the product of interest complexed with the binding molecule (e.g., monoclonal antibody bound to Protein A) can be dissociated with low pH. In some aspects, the Protein A can diffuse through the semi-permeable membrane.

In some aspects, the product of interest purification methods can be modularly combined. In some aspects, the product of interest (e.g., monoclonal antibody) obtained from a bioreactor can be first subjected to the Protein A affinity colloid separation described herein. The product of interest (e.g., monoclonal antibody) can then further be subjected to a cation exchange polymer affinity colloid separation, followed by an anion exchange polymer affinity colloid separation, followed by a diafiltration step.

In some aspects, dirty product (e.g., product of interest) is obtained from a bioreactor and one of the separation methods described herein is used (e.g., affinity colloid separation, anion exchange polymer affinity colloid separation, etc.) to concentrate and purify the product (e.g., product of interest). A second separation method is then performed on the concentrated product. The dirty permeate from the second separation is fed back into the dirty product stream. The concentrated product (e.g., product of interest) from the second separation method is then diafiltrated. The slightly dirty permeate from the diafiltration step is fed back into the concentrated product (e.g., product of interest) from the first separation method.

In some aspects, the methods disclosed herein can be performed at elevated temperatures, due to increased diffusivity and decreased viscosity. In some aspects, the methods described herein can be performed at temperatures of about 37° C., about 38° C., about 39° C., or about 40° C. In some aspects, semi-permeable ceramic membranes are used in the methods described herein when performed at temperatures of about 37° C., about 38° C., about 39° C., or about 40° C.

In some aspects, semi-permeable ceramic membranes can be used in dialysis or diafiltration/ultrafiltration modes. These have the advantage of multiple cleaning and use compared to polymeric based semipermeable membranes.

In some aspects, semi-permeable membrane fouling can be reduced by a variety of methods, including but not limited to, back flushing, forward flushing, pulsed operation, ultrasonic fouling reduction, gas entrainment, and/or electrophoretic fouling reduction.

In some aspects, high performance tangential flow filtration (HPTFF) can also be used for the diafiltration/ultrafiltration. HPTFF is known to those skilled in the art as an ultrafiltration operation where the permeate is recycled co-currently with the retentate to maintain a more consistent trans-membrane pressure.

In some aspects, the methods herein can be performed with the first solution comprising the product of interest having concentrations of product of interest of about 20 g/L, about 25 g/L, about 30 g/L, about 35 g/L, about 40 g/L, about 45 g/L, about 50 g/L, about 55 g/L, about 60 g/L, about 65 g/L, about 70 g/L, about 75 g/L, about 80 g/L, about 85 g/L, about 90 g/L, about 95 g/L, about 100 g/L, about 110 g/L, about 120 g/L, about 130 g/L, about 140 g/L, about 150 g/L, about 160 g/L, about 170 g/L, about 180 g/L, about 190 g/L, about 200 g/L, or in excess of 200 g/L.

### Dialysis Effluent

In some aspects, dialysate solution is acceptable for using upstream in a previous step. In some aspects, the dialysate solution is titrated to a new pH and/or mixed with a high concentration salt solution to achieve the desired ionic strength for use in an upstream step. In some aspects, other solutions are added to the dialysate solution, including but not limited to, solutions of arginine, urea, guanidine, ethanol, isopropyl alcohol, other alcohols, caprylic acid, and other solutions suitable in washing the product of interest. In some aspects, this system runs continuously. In some aspects, the dialysate solution is stored in a small surge tank. In some aspects, the surge tank is smaller than one day's volume of solution. In some aspects, recycling of solutions from a downstream step to an upstream step requires the use of a surge tank.

In some aspects, the permeate from an ultrafiltration step can also be used in a countercurrent manner from step to step (e.g., as described herein). In some aspects, the permeate from a single pass tangential flow filtration step can also be used in a countercurrent manner from step to step (e.g., as described herein). In some aspects, the dialysate from a dialysis step can be used in a countercurrent manner, from step to step. In some aspects, the retentate from a dialysis step can also be used in a countercurrent manner, from step to step.

In some aspects, the previous upstream step that receives the countercurrent solution, or a step after the receiving step, must have an effluent to waste that facilitates the removal of any impurities that can be in the recycled solution. In some aspects, if the recycled solution comes from the permeate of a step that has a semi-permeable membrane with a molecular weight cut-off (MWCO), the receiving step of a subsequent step must have a membrane with the same MWCO or larger that is directed to waste. If not, even small quantities of material larger than the upstream step's MWCO can build up on the system over time. In some aspects, the upstream step can have a semi-permeable membrane with a MWCO greater than the downstream step, such that any large material that passes through the downstream filter is able to easily pass through the upstream filter to the waste stream.

In some aspects, if the recycled solution comes from the retained portion of a semi-permeable membrane, the receiving step must have a semi-permeable membrane the same size or smaller than the MWCO of the downstream step, and the retentate of the upstream step must go to waste.

In some aspects, all combinations of recycling are contemplated, from the retained or permeate portion of the downstream step, to the upstream step, where the upstream step (or a step subsequent to the upstream step yet prior to the downstream step) has a waste stream on the retained portion of the permeate portion of the filter.

In some aspects, the build up of trace materials in a recycled loop can be taken care of by diverting a portion the recycle line to waste, or by periodically emptying the recycle loop.

In some aspects, if the step is an anion exchange step, the receiving step or a subsequent step must have an effluent that removes anionic compounds. In some aspects, if the step is a cation exchanges step, the receiving step must have an effluent that removes cationic compounds. For example, if the downstream step involves an anion exchange nanoparticle or polymer, cationic components will flow through the permeate or dialysate of the membrane. If this material is used upstream, the upstream step must allow a fraction of the cationic components flow go to drain. This can be done with a cation exchange nanoparticle or polymer that binds retains cationic compounds on the retentate side, while effluent flowing to the downstream step comes from the permeate. Or, the upstream step could be an affinity step, which is orthogonal to ion exchange, and the cationic components would flow through the affinity step to the waste line.

In some aspects, in the absence of such a removal step upstream, even trace levels of an impurity can build up in the recycle loop. In some aspects, this build up can be managed by a split stream, where a portion of the recycle loop goes to waste. This split stream is less efficient method, as product is also directed to waste.

In some aspects, the present disclosure is directed to a method of using a solution effluent from a continuous downstream step as a wash for an upstream step. In some aspects, the solution effluent is mixed with a solution stream to adjust the pH, or salt concentration, or other excipient concentration to a desired level.

In some aspects, the present disclosure is directed to a method of using a solution effluent from the filtrate or dialysate of a continuous downstream step as a wash for an upstream step, wherein the upstream step has a filter with a MWCO the same size or larger than the downstream step that generated the effluent.

In some aspects, the present disclosure is directed to a method of using a solution effluent from the retentate of a continuous downstream step as a wash for an upstream step, wherein the upstream step has a filter with a MWCO the same size or smaller than the downstream step that generated the effluent, and the filtrate is directed downstream.

In some aspects, the present disclosure is directed to a method of using a solution effluent from a continuous downstream step as a wash for an upstream step, where the downstream step retains anions, and the upstream step allows cations to go to waste.

In some aspects, the present disclosure is directed to a method of using a solution effluent from a continuous downstream step as a wash for an upstream step, wherein the downstream step retains cations, and the upstream step allows anions to go to waste.

In some aspects, the present disclosure is directed to a method of using a solution effluent from a continuous downstream step as a wash for an upstream step, wherein the downstream step retains certain classes of impurities, and the upstream step allows these impurities to go to waste.

In some aspects, the upstream step is a concentration step for an Fc-containing protein, and the upstream step is a continuous Protein A wash step.

In some aspects, the present disclosure provides an effluent stream from a continuous concentration step in the recycle of the binding molecule to wash the complex of the binding molecule and the product of interest. In some aspects, a titrant, salt or other excipients are added to the effluent stream. In some aspects, the dialysis or ultrafiltration step is continuous. In some aspects, the effluent does into a small tank, wherein the tank size is less than one day's storage of effluent. In some aspects, the effluent foes into a small tank, wherein the tank size is less than three hours storage of effluent.

In some aspects, the recycling of binding molecule are Protein A affinity ligands.

In some aspects, the present disclosure is directed to a method of using the solution effluent from a dialysis step or a ultrafiltration concentration step in a step upstream of the dialysis or ultrafiltration step. In some aspects, a titrant, salt, or other excipient is added to the effluent. In some aspects, the dialysis or ultrafiltration step is continuous. In some aspects, the effluent does into a small tank, wherein the tank size is less than one day's storage of effluent. In some aspects, the effluent does into a small tank, wherein the tank size is less than three hours storage of effluent. In some aspects, the product of interest is a protein, DNA, RNA, a virus or virus like particle, a synthetic molecule, or part of an affinity complex.

### ProA Recycle SPTFF Effluent

Typically, the stream of affinity moiety taken from the step that separates the affinity ligand from the product of interest is dilute. A semi-permeable membrane with a MWCO lower than the MW of the affinity ligand can be used to concentrate the affinity ligand prior to recycling it. The permeate or dialysate from this concentration step can be used in a counter current mode as part of the wash for the affinity step. This solution can be titrated or mixed with other solutions which may have a high concentration of salt or other compounds related helpful to remove impurities in the wash.

In some aspects, the present disclosure is directed to a method of using an effluent stream from a continuous concentration step in the recycle of the affinity moiety to wash the complex of the affinity moiety and the product of interest. In some aspects, the method further comprises adding a titrant, salt, or other excipient to the effluent stream. In some aspects, the dialysis or ultrafiltration step is continuous. In some aspects, the effluent does into a small tank, wherein the tank size is less than one day's storage of effluent. In some aspects, the effluent goes into a small tank, wherein the tank size is less than three hours storage of effluent. In some aspects, the binding molecules are Protein A. In some aspects, the product of interest has a HIS tag. In some aspects, the Protein A is larger than 500 kDa. In some aspects, the Protein A is smaller than 50 kDa.

### Binding Molecule

In some aspects, when the binding molecule (e.g., also referred to as affinity moiety) is a recombinant protein, and the product of interest is a recombinant protein, the binding molecule can be made in the same cell expression system as the product of interest. In some aspects, the growth media used to generate the binding molecule is the same as the growth media used to generate the product of interest. This method of producing the binding molecule reduces the overall cost of the binding molecule, since the impurities in the binding molecule are the same as that in the product of interest. This similarity of the impurities means that the binding molecule (e.g., affinity moiety) can be purified so that it is cleaner than the step at which the binding molecule (e.g., affinity moiety) and product of interest are mixed. When this step is the capture step, and the product of interest has a great degree of impurities, the binding molecule can be dirtier than one would expect, because the step will purify the binding molecule/product of interest complex.

For example, the product of interest has well over 100,000 ppm of host cell proteins (HCPs) upon capture. The binding molecule can have 1,000 ppm or even 10,000 ppm HCP and still purify the product of interest with minimal impact. Typically, one would expect the binding molecule to have a purity of less than 100 ppm or even less than 10 ppm of HCP. The higher the allowable impurity level for the binding molecule, the cheaper it is to manufacture.

The initial purification of the binding molecule in such a system is relatively easy. If the binding molecule is larger than the product of interest, the binding molecule can be passed through a series of the same filters as used in with the product of interest. The binding molecule is first concentrated by a asymmetric dialysis or single-pass TFF step, then washed in a dialysis or CM-TFF step. The material is finally filtered through a CM-TFF step or a single pass TFF step with a MWCO or pore size larger than the of the affinity moiety.

If the binding molecule is smaller than the product of interest, then it can be purified through the use of a system similar to the “elution” step of the affinity/target cycle.

### Wash Solutions

In some aspects, suitable solutions for washing the product of interest during the one or more wash steps comprise solutions made from arginine, urea, guanidine, ethanol, isopropyl alcohol, other alcohols, caprylic acid, and other solutions suitable.

### Design of Binding Molecule (e.g., Affinity Moiety)

In some aspects, the binding molecule has two unique characteristics. First, it is designed to be easily separable from the product of interest in non-binding conditions. Second, the complex formed between the binding molecule and product of interest contains two or more products of interest (e.g., monoclonal antibodies) under binding conditions. In some aspects, the number of products of interest per complex is 3, 4, 5, 6, 7, 8, 9, 10, 11, 12, 13, 14, 15, 16, 17, 18, 19, 20, or more than 20. In some aspects, the molar ratio of the binding molecule to the product of interest is about 1:2 or 1:3 or higher. In some aspects, the binding molecule is capable of binding twice or more its weight in product of interest. In some aspects, the mass ratio of the binding molecule to the product of interest is about 1:2, about 1:3, about 1:4, about 1:5, about 1:6, about 1:7, about 1:8 about 1:9, or about 1:10.

In some aspects, the binding molecule is to be easily separable from the product of interest under “elution” or non-binding conditions. For instance, Fc-containing moieties elute or cease binding to Protein A when the pH is lowered to approximately 3.5.

In some aspects, the Protein A (e.g., binding molecule) can be constructed to be significantly smaller than the Fc-containing compound (e.g., product of interest). In some aspects, for ultrafiltration separations, this means that the Protein A (e.g., binding molecule) has to be ⅕^{th} to 1/10^{th} the size of the Fc-containing compound (e.g., product of interest). In some aspects, for dialysis separations, acceptable purification can occur if the Protein A is less than ⅓ the size of the Fc-containing compound. In some aspects, if the product of interest is a typical antibody (150 kDa MW), then the Protein A moiety should be less than 30 kDa. A 100 kDa MWCO membrane is sufficient to retain the antibody and allow significant portion of the affinity ligand to pass through. Native Protein A has a MW of between 40-60 kDa, which is about ⅓ that of an antibody. However, native Protein A cannot be separated with a ultrafiltration or dialysis device. Protein A is either retained with the antibody, or they both go through the membrane.

For example, a single domain of Protein A can bind an antibody in solution, but it does not create a complex of two or more antibodies, and the ultrafiltration or diafiltration of a crude feedstream does not remove much more impurities than the same system and just the unbound antibody.

In some aspects, a Protein A construct with two binding domains can bind two antibodies (e.g., product of interest), which can allow a complex to form that is filterable. Under elution conditions, some of the Protein A construct can pass through a ultrafilter or diafilter with a 100 kDa MWCO or a 75 kDa MWCO while retaining the antibody.

In some aspects, the Protein A can be constructed to be significantly larger than the Fc-containing compound (e.g., product of interest). For example, a Protein A moiety greater than 500 kDa can be created. A 500 kDa membrane is sufficient to allow the antibody to flow through the membrane by convection while retaining the affinity ligand. A 300 kDa membrane can also allow passage of the antibody, but with less efficiency. In dialysis, a 300 kDa membrane is sufficient to allow the antibody to diffuse through. Larger MWCO membranes also are efficient.

In some aspects, a binding molecule made from a peptide or protein can have a his-tag (typically a six-histidine tag on the amino or carboxy end of the protein). If the elution conditions are above pH 7, divalent cations such as copper or cobalt can be added to aggregate the binding molecule, allowing filtration. At pH 4 or 5, the his tag becomes positively charged, and cation exchange methods can be used to separate the binding molecule from the product of interest.

In some aspects, a product of interest comprising DNA can have a sequence that specifically binds a large nanoparticle or microparticle, thus allowing the separation to occur.

In some aspects, a binding molecule comprises elastin-like peptide sequences that allow spontaneous aggregation under certain conditions.

In some aspects, a binding molecule comprises multiple possible built-in ways of allowing a separation under elution/non-binding conditions.

In some aspects, a binding molecule comprising a small peptide can be found, for instance by phage display screening, for small molecular weight synthetic or naturally occurring molecules. In some aspects, the peptide can be separated from the small molecular weight compound if it is significantly larger than the compound under non-binding conditions. Most peptides would be larger than the small molecule targets mentioned above.

In some aspects, the binding molecules must have an affinity constant much less than the concentration of the product of interest in the solution.

In some aspects, polymers can be used as binding molecules. In some aspects, the polymers are ionic and/or hydrophobic.

In some aspects, the complex formed by the binding molecule and product of interest contains at least two products of interest per complex or at least three products of interest per complex. In some aspects, the size of the complex is significantly larger than the product of interest itself to facilitate separation across a semi-permeable membrane.

In some aspects, a Protein A molecule (e.g., binding molecule) can bind three antibodies (e.g., product of interest) and be purified on a 300 kDa membrane or a 500 kDa membrane. There is passage of the complex through a 700 kDa membrane.

In some aspects, a very large Protein A (e.g., binding molecule) could bind one antibody (e.g., product of interest) and be retained by a semi-permeable membrane, but the cost of the protein A is prohibitive. Further, the separation of the large MW Protein A from the antibody becomes more challenging, because the concentration of the Protein A is high, and it is retained by the membrane. The retention of the high concentration Protein A causes the semi-permeable membrane to have reduced performance. The flux goes down, the resistance goes up, the antibody can be more highly retained. Thus, a low concentration of Protein A compared to Antibody is preferred. Thus, in some aspects, the ratio of antibody to protein A is 2:1 or more preferably 3:1 mole per mole. In some aspects, for very large Protein A moieties, such as those higher than 500 kDa, the molar ratio would be 4:1 or 8:1 or even 20:1. A 8:1 molar ratio is a mass ratio of 2.4:1 antibody:Protein A. higher mass ratios are preferred due to cost considerations and filter efficiency.

In some aspects, a very small Protein A molecule (e.g., binding molecule) should efficiently bind at least two antibodies (e.g., product of interest), such that a chain reaction can occur and a complex of three or more antibodies can be formed. In some aspects, the size of the complex can be controlled with the addition of Protein A molecules that bind only one antibody, creating a “dead-end’ in the chain reaction. The size of the complex should not be too high, as the complex becomes difficult to filter. In some aspects, for Protein A/mAb complexes, the size of the complex can be 300 kDa, 500 kDa, 700 kDa, 1 MDa. In some aspects, the size can be as small as 70 nm and as large as 1 um and the complex filtered out by an 0.2 um filter. In some aspects, the size may be 10 um or 100 um. The cost of microfilters is typically cheaper than nanofilters, so the largest filterable particle is preferred. A 300 kDa particle does allow economic purification, even though a 300 kDa membrane is more expensive than a 0.2 um membrane.

In some aspects, a very small Protein A molecule (e.g., binding molecule) can bind one antibody (e.g., product of interest), and it can contain another affinity domain that binds to another Protein A molecule, that itself is bound to an antibody. Thus, in some aspects, a large complex can be formed. For instance, in some aspects, a single domain Protein A can have a his-tag that, in the presence of chelated metal ions such as cobalt, copper, nickel or others, can bind the Protein A domains to one another, such that a complex of Protein A and antibody is formed. His-tagged proteins are known to those skilled in the art of protein expression and purification. Without being restricted, the binding molecule, in some aspects, a Protein A domain, can be tagged with 3-6 histidine. In some aspects, the molar ratio of Protein A to mAb is about 2:1, but the mass ratio is quite high, around 1:10, or 1:15 or even 1:30. In some aspects, the size of the complex can be controlled with the addition of Protein A domains without his-tags. In some aspects, the ratio of non-his-tagged to his-tagged Protein A can be 1:2 (which creates relatively small particles), 1:3, 1:5, 1:10 or higher. In some aspects, the more his-tagged Protein A relative to the non-his-tagged Protein A will generate larger and larger particles. In some aspects, the larger particles create difficult to filter particles. In some aspects, the preferred ratio is 1:3 to 1:5. The single-domain Protein A (with or without histidine) can be separated by ultrafiltration or dialysis with a 50 kDa membrane, more preferably with a 75 kDa, and most preferably with a 100 kDa membrane. The his-tagged Protein A passes through the membrane while the antibody is retained. In some aspects, the chelating metal is added to the Protein A before the antibody, and in some aspects, the antibody is bound to the Protein A, and then the chelating metal is added.

The methods of the present disclosure may also involve the use of a self-assembling nanoparticle. Such a nanoparticle would have the capacity to assemble and bind multiple antibodies simultaneously, creating a large complex. In some aspects, the binding molecule is a nanoparticle. Self-assembling nanoparticles are formed from the self-association of the monomers encoded in each plasmid. The number of monomers that assemble into the nanoparticle varies (e.g. about 2, about 5, about 12, about 24-60 or more). The MW of the fusion nanoparticle is the product of monomer size and number of monomers in the nanoparticle while the MW of the mAb bound species is the product of monomer MW, mAb MW, number of monomers in the nanoparticle, % capacity (mol mAb/mol ProA Z binding domains). While the nanoparticle, present on the retentate side of a filtration membrane after elution, should have an apparent MW larger than the cutoff of the elution membrane filter (e.g. 300 or 500 kDa MWCO), the affinity bound mAb-nanoparticle complex should have a MW larger the cutoff of the wash and binding membrane filter (e.g. 500, 750, 1000 kDa MWCO or 0.22 um). Optional additional Protein A Z-domains add mass to the nanoparticle and additional mass to the mAb-nanoparticle complex. Similarly, mass ratio of bound mAb to nanoparticle increases with number of Z-domains only if additional mAb can bind.

Self-assembling nanoparticles span various sizes, number of comprising homo-monomers, and sequences. Ferritin nanoparticles form a 12 nm pore containing hollow sphere composed of 24 monomers, each containing 5 helical domains. It was selected for its pH resistance <4 required to maintain its structure in the face of conditions used to dissociate mAb from an affinity ligand. Helicobacter pylori ferritin above was modified from the WT form to include amino acids at its N terminus that would allow for a favorable spatial separation of N-termini that would be modified with an affinity ligand. Stable Protein 1 (SP1) exhibits high temperature stability and pH stability, is composed of 12 subunits that have been shown as reliable supports for enzymes and carbon nanotube binding proteins which formed bio-materials. SP1 also has a feature that it can reversibly form nano-rods which may be favorable for mAb (and nanoparticle) size retention over large pore sizes.

In aspects of the methods, the fusion protein monomer comprises: i) a self-assembling nanoparticle monomer; ii) a linker; and iii) an immunoglobulin binding domain. In aspects, the linker connects the self-assembling nanoparticle monomer and the immunoglobulin binding domain.

In aspects, the methods further comprise regenerating the fusion protein monomer, wherein the regenerated fusion protein monomer is again capable of forming an assembled nanoparticle and a complex upon contact with the protein of interest in the first flow solution or the second flow solution.

In aspects of the methods, the self-assembling nanoparticle monomer is a ferritin monomer. In aspects of the fusion protein monomers, the ferritin monomer is a Helicobacter pylori ferritin monomer or a Pyrococcus furiosus ferritin monomer. In aspects, the ferritin monomer is a Helicobacter pylori ferritin monomer. In aspects, the ferritin monomer is a Pyrococcus furiosus ferritin monomer. In aspects, the ferritin monomer is a human ferritin monomer. In aspects, the ferritin monomer is a human H-chain ferritin monomer. In aspects, the ferritin monomer is a human L-chain ferritin monomer. In aspects, the ferritin monomer is a ferritin monomer disclosed in Table 1 below.

In aspects of the methods, the ferritin monomer comprises an amino acid sequence at least 90%, 95%, 98% or 100% identical to SEQ ID NO: 1. In aspects, the ferritin monomer comprises an amino acid sequence at least 90%, 95%, 98% or 100% identical to SEQ ID NO: 1. In aspects, the ferritin monomer comprises an amino acid sequence at least 90%, 95%, 98% or 100% identical to SEQ ID NO: 2. In aspects, the ferritin monomer comprises an amino acid sequence at least 90%, 95%, 98% or 100% identical to SEQ ID NO: 2.

In aspects, the assembled nanoparticle is an assembled ferritin nanoparticle. In aspects, the assembled ferritin nanoparticle comprises any of the ferritin monomers discussed above. In aspects, the assembled ferritin nanoparticle comprises more than one type of the ferritin monomers discussed above.

In some aspects, the bispecific nature of the small molecular binding molecule does not have to bind the same site on the product of interest. One can create a complex of two or more products of interest by binding to two different sites on the product of interest. In some aspects, this heterogeneous bispecific affinity is preferred for molecules that do not have two binding sites on them. Antibodies can bind two Protein A molecules because antibodies are dimers, so that the complex can get larger. Most molecules will not have two binding sites for affinity, so the bispecific nature of the affinity ligand requires two different affinity moieties on the same site.

In some aspects, a bispecific peptide can be created from joining together two affinity peptides such that the combined peptide binds to two different epitopes on a small synthetic or natural target, thus allowing a larger complex to be found and purified by a nanofiltration system such as those described. In some aspects, MWCO are expected to be much lower than for proteins, DNA, RNA, or viruses or virus like particles. MWCO near 500 Da, 1 kDa, 2 kDa, 4 kDa, 8 kDa, 16 kDa, 32 kDa can be useful in such a system.

In some aspects, an affinity moiety made of a small DNA or RNA strand can be found, for instance by common screening methods, for small molecular weight synthetic molecules. A bispecific moiety can be created from two different affinity moieties that binds to two different epitopes on a small synthetic target, thus allowing a larger complex to be found and purified by a nanofiltration system such as those described. In some aspects, MWCO are expected to be much lower than for proteins, DNA, RNA, or viruses or virus like particles. MWCO near 500 Da, 1 kDa, 2 kDa, 4 kDa, 8 kDa, 16 kDa, 32 kDa may all be useful in such a system.

In some aspects, a binding molecule made of a small peptide can be found, for instance by phage display screening, for small molecular weight synthetic or naturally occurring molecules. Some targets might include aspirin or a metabolite like those discussed in Mulukutla et al, Biotech Bioeng, V 114, 2017, which inhibit CHO cell growth. These affinity ligands must have an affinity constant much less than the concentration of the product in the solution. In some aspects, the peptide can be separated from the small molecular weight compound if it is significantly larger than the compound under non-binding conditions.

In some aspects, the present disclosure provides a binding molecule that has two or more binding sites, wherein each of the binding sites binds to an epitope on a product of interest, and the complex comprising the binding molecule and product of interest is larger than the product of interest. In some aspects, the complex is about 25% larger, about 50% larger, about 100% larger, about 200% larger, or about 400% larger than the product of interest. In some aspects, the complex comprises one binding molecule and about two or more products of interest. In some aspects, the complex loses affinity for the product of interest under specific elution conditions. In some aspects, the binding molecule has 2, 3, 4, 5, 6, 7, 8, 9, or 10 binding sites for the product of interest. In some aspects, the binding molecule comprises of two or more Protein A binding domains with a molecular weight of about 300 kDa, about 400 kDa, about 600 kDa, or about 1,000 kDa. In some aspects, the two or more Protein A binding domains are capable of binding the mass of the target Fc-containing molecule twice the mass of the Protein A. In some aspects, the complex comprises three or more fab-fragments with a molecular weight of abut 100 kDa, about 300 kDa, about 600 kDa, or about 1,000 kDa. In some aspects, the complex comprises DNA with two or more affinity sites with a molecular weight of about 300 kDa, about 400 kDa, about 600 kDa, or about 1,000 kDa. In some aspects, the complex comprises peptides with two or more binding sites with a molecular weight of about 1 kDa, about 2 kDa, about 4 kDa, about 8 kDa, or about 16 kDa. In some aspects, the complex comprises RNA with two or more affinity sites with a molecular weight of about 300 kDa, about 400 kDa, about 600 kDa, or about 1,000 kDa.

In some aspects, the filtration is continuous dialysis. In some aspects, the filtration is continuous modular countercurrent tangential flow filtration. In some aspects, the complex is separated into product of interest and binding molecule by filtration under specific elution conditions, and the MWCO of the membrane or the pore size of the membrane is smaller than the binding molecule and larger than the product of interest.

In some aspects, the binding molecule has two or more binding sites for a product of interest, wherein one of the sites binds to a first target of interest, and the other site binds to a second product of interest. In some aspects, the binding molecule has a molecular weight smaller than the product of interest. In some aspects, a second binding molecule only has one of these binding sites. In some aspects, a mixture of the binding molecules and the product of interest create a complex with a weight ratio of at least one unit binding molecule and about two or more units of product of interest. In some aspects, the ratio of binding molecule to complex is controlled by the size of the complex. In some aspects, the ratio binding molecule to product of interest is about 1:3, about 1:5 or about 1:10. In some aspects, filtration is of the complex is used to purify the product of interest. In some aspects, the filtration is continuous dialysis. In some aspects, the filtration is continuous modular countercurrent tangential flow filtration. In some aspects, the binding molecules and the product of interest are separated by filtration under elution conditions, wherein the binding molecules pass through the filer while the product of interest is retained. In some aspects, the filter area is greater than 1 m². In some aspects, the filtration requires the addition of a polymer or particle larger than the molecular weight cut off of the filter being used, that binds to the affinity moiety.

In some aspects, the binding molecule has two or more binding sites, wherein one of the sites binds to one of two similar epitopes on a product of interest, and the other site binds to one of two similar epitopes on a separate product of interest. In some aspects, a separate binding molecule only has one of these binding sites. In some aspects, the complex comprises at least one binding molecule and two or more products of interest. In some aspects, this ratio is controlled by the size of the complex. In some aspects, the ratio is about 1:3, about 1:5, or about 1:10. In some aspects, the complex is filtrated to purify the product of interest. In some aspects, the filtration is continuous dialysis. In some aspects, the filtration is modular countercurrent tangential flow filtration. In some aspects, the binding molecule and the product of interest are separated by filtration under elution conditions. In some aspects, the filtration requires the addition of a polymer or particle larger than the MWCO of the filter being used, that binds to the binding molecule. In some aspects, the binding molecule has a molecular weight three times larger than the product of interest.

### Dialysis Based Affinity Separations

In some aspects, the binding molecule can be placed in the dialysate solution if the size of the binding molecule is larger than the MWCO of the dialysis membrane, and the product of interest is smaller than the MWCO of the membrane. In some aspects, the product of interest flows into the dialysis device on the retentate side of the filter, and the larger binding molecule is suspended in the dialysis solution on the other side of the membrane, in a countercurrent flow to the product of interest flow. The unbound large binding molecule cannot diffuse across the membrane to the retentate side because it is larger than the MWCO. The product of interest diffuses through the membrane and binds to the large binding molecule. After binding, it cannot diffuse or convect back to the retentate side, because the complex is too large.

In some aspects, a dialysis process uses a higher flux of solution in the clean dialysis side of the membrane compared to the retentate side of the membrane to allow diffusion. The alpha, or ratio of the dialysis flow rate to the retentate flow rate, is typically 2, 4, 8, or 16 times larger than the retentate flow rate.

The addition of a large binding molecule to the dialysis solution allows the flow rate of the dialysis solution to be much lower than without. The ratio of useful flow rates, or alpha, is proportional to the affinity constant and inversely proportional to the concentration of the affinity ligand. Some equilibrium models can apply, like the Langmuir isotherm. For example, in some aspects, in a system that has a MWCO of 500 kDa, or 700 kDa, or 1000 kDa, a large Protein A moiety can be used to maintain the concentration of the target equal to that in the retentate (alpha=1), or more preferably, to decrease the alpha to 1/2, 1/4, 1/8, 1/16, 1/32, 1/64 or 1/128, and therefore increase the concentration of an antibody from the retentate to the dialysisate by a factor of 2, 4, 8, 16, 32, 64, or 128 times.

Under flow conditions, the amount of product of interest going through the semi-permeable membrane is related to the diffusivity of the product of interest in the membrane, the affinity and concentration of the binding molecule on the dialysis side of the membrane, and the area of the membrane, and inversely to the thickness of the membrane, among other factors. In some aspects, the mass transport of the product of interest in the lumen can impact the amount of product of interest going through the membrane, especially when the thickness of the retentate flow field is much greater than the thickness of the membrane (e.g., in the case of hollow fibers, this is when the hollow fiber lumen is much bigger than the membrane). Methods to induce radial flow in the lumen of the membrane, or orthogonal flow in the case of a flat sheet, are known. Similarly, the mass transport may be impacted by the flow distribution of the dialysis fluid. In some aspects, methods to provide radial flow, to allow the dialysate material to reach the inner part of a bundle of hollow fiber membranes, are used. In some aspects, these methods include spacing the hollow fibers on the top and bottom of the cartridge to allow radial flow, using dean vortices, using pulsed flow, using gas bubbles to provide radial mixing. In some aspects, kinked membranes or twisted membranes can also provide radial mixing. In some aspects, in the case of flat sheets many of these same methods can be used. In some aspects, screens can also be used to create mixing orthogonal to the membrane.

### III. Product of Interest

In some aspects, the methods disclosed herein can be applied to any protein product (e.g., a product of interest). In some aspects, the protein product is a therapeutic protein. In some aspects, the therapeutic protein is selected from an antibody or antigen-binding fragment thereof, an Fc fusion protein, an anticoagulant, a blood clotting factor, an engineered protein scaffold, an enzyme, a growth factor, a hormone, an interferon, an interleukin, a receptor, and a thrombolytic. In some aspects, the protein product is an antibody or antigen-binding fragment thereof. In some aspects, the protein is a recombinant protein.

In some aspects, the protein product is an antibody or an antigen binding fragment thereof. In some aspects, the protein product is a chimeric polypeptide comprising an antigen binding fragment of an antibody. In certain embodiments, the protein product is a monoclonal antibody or an antigen binding fragment thereof (“mAb”). The antibody can be a human antibody, a humanized antibody, or a chimeric antibody. In certain embodiments, the protein product is a bispecific antibody.

In some aspects, a mixture comprising the protein product and the contaminant comprises a product of a prior purification step. In some aspects, the mixture is the raw product of a prior purification step. In some aspects, the mixture is a solution comprising the raw product of a prior purification step and a buffer, e.g., the starting buffer. In some aspects, the mixture comprises the raw product of a prior purification step reconstituted in the starting buffer.

In some aspects, the source of the protein product is bulk protein. In some aspects, the source of the protein product is a composition comprising protein product and non-protein components. The non-protein components can include DNA and other contaminants.

In some aspects, the source of the protein product is from an animal. In some aspects, the animal is a mammal such as a non-primate (e.g., cow, pig, horse, cat, dog, rat etc.) or a primate (e.g., monkey or human). In some aspects, the source is tissue or cells from a human. In certain aspects, such terms refer to a non-human animal (e.g., a non-human animal such as a pig, horse, cow, cat or dog). In some aspects, such terms refer to a pet or farm animal. In some aspects, such terms refer to a human.

In some aspects, the protein products purified by the methods described herein are fusion proteins. A “fusion” or “fusion protein” comprises a first amino acid sequence linked in frame to a second amino acid sequence with which it is not naturally linked in nature. The amino acid sequences which normally exist in separate proteins can be brought together in a fusion polypeptide, or the amino acid sequences which normally exist in the same protein can be placed in a new arrangement in the fusion polypeptide. A fusion protein is created, for example, by chemical synthesis, or by creating and translating a polynucleotide in which the peptide regions are encoded in the desired relationship. A fusion protein can further comprise a second amino acid sequence associated with the first amino acid sequence by a covalent, non-peptide bond or a non-covalent bond. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In a particular aspect, the fusion protein further comprises a third polypeptide which, as discussed in further detail below, can comprise a linker sequence.

In some aspects, the proteins purified by the methods described herein are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In some aspects, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In some aspects, antibodies described herein are IgG antibodies, or a class (e.g., human IgG1 or IgG4) or subclass thereof. In a aspects, the antibody is a humanized monoclonal antibody. In some aspects, the antibody is a human monoclonal antibody, preferably that is an immunoglobulin. In some aspects, an antibody described herein is an IgG1, or IgG4 antibody.

The present disclosure is directed to methods disclosed herein, wherein the product of interest is an antibody, an antigen-binding antibody fragment, a fusion protein, a naturally occurring protein, a chimeric protein, or any combination thereof. In some aspects, the product of interest is a full length IgG antibody. In some aspects, the antibody is an IgG1, IgG2, IgG3, and/or IgG4, or hybrids thereof. In some aspects, the antibody is a monoclonal antibody.

In some aspects, the methods disclosed herein are accomplished using bacterial cells, yeast cells, insect cells, or mammalian cells. In some aspects, the mammalian cells are Chinese hamster ovary cells. In some aspects, the product of interest are prepared by the methods disclosed herein.

SEQUENCES

| SEQ | | |
|---|---|---|
| ID | | |
| NO: | Sequence | Description |
| 1 | ESQVRQQFSKDIEKLLNEQVNKEMQSSNLYMSMSSWCYTHSL | Helicobacter pylori |
| | DGAGLFLFDHAAEEYEHAKKLIIFLNENNVPVQLTSISAPEHKF | ferritin |
| | EGLTQIFQKAYEHEQHISESINNIVDHAIKSKDHATFNFLQWY | |
| | VAEQHEEEVLFKDILDKIELIGNENHGLYLADQYVKGIAKSRK | |
| | S | |
| 2 | MLSERMLKALNDQLNRELYSAYLYFAMAAYFEDLGLEGFAN | Pyrococcus furiosus |
| | WMKAQAEEEIGHALRFYNYIYDRNGRVELDEIPKPPKEWESP | ferritin |
| | LKAFEAAYEHEKFISKSIYELAALAEEEKDYSTRAFLEWFINEQ | |
| | VEEEASVKKILDKLKFAKDSPQILFMLDKELSARAPKLPGLLM | |
| | QGGE | |
| 3 | MATRTPKLVKHTLLTRFKDEITREQIDNYINDYTNLLDLIPSM | Stable Protein 1 |
| | KSFNWGTDLGMESAELNRGYTHAFESTFESKSGLQEYLDSAA | (SP1) |
| | LAAFAEGFLPTLSQRLVIDYFLY | |
| 4 | VDNKFNKEQQNAFYEILHLPNLNEEQRNAFIQSLKDDPSQSAN | Protein A Z (WT) |
| | LLAEAKKLNDAQAPK | |
| 5 | VDNKFNKEQHHAFYEILHLPNLNEEQRNAFIQSLKDDPSQSAN | Protein A Z (HH) |
| | LLAEAKKLNDAQAPK | |
| 6 | VDNKFNKEQHHAFYEILSLPNLNEEQRAAFIQSLKDDPSQSAN | Protein A Z (HH*) |
| | LLAEAKKLNDAQAPK | |
| 7 | VDNKLNKEQQNAFYEILHLPNLNEEQRKAFIQSLIDGGGDING | Protein A Z (Ca) |
| | NGYLDAEESANLLAEAKKLNDARAPK | Zmat-8 |
| 8 | AGSGGSGGSGGSPVPSTPPTPSPSTPPTPSPSPVPSTPPTPSPSTP | Un-glycosylated |
| | PTPSPSPVPSTPPTPSPSTPPTPSPSAS | polyproline-rich |
| | | linker |
| 9 | ASGAPGPGGAGPGGAGPGGAGPGGAGPGGAGPGGAGPGGA | Spider silk derived |
| | GPGGAGAGRSSG | spring linker |
| 10 | ASGASMALEKMALEKMALEKGSMALEKMALEKMALEKMA | Alpha helix linker |
| | LEKMALEKGSMALEKMALEKMALEKPSGRSSG | |
| 11 | ASGAPGPGGAGPGGAGPGGAGPGGAGPGGAGPGGAGPGGA | Alpha helix with |
| | GPGGAGAGRNASMALEKMALEKMALEKGSMALEKMALEK | spring |
| | MALEKMALEKMALEKGSMALEKMALEKMALEKPSGRSSG | |
| 12 | PPPP | Rigid hydrophobic |
| | | linker |
| 13 | EKEKEKEPPPP | Mixed Flexible and |
| | | Rigid |
| 14 | GAAGCTGCTGCTAAAGAAGCTGCTGCTAAAGAAGCTGCTG | Rigid linker |
| | CTAAA | |
| 15 | LFNKEQQNAFYEILHLPNLNEEQRNGFIQSLKDDPSQSANLLA | B Domain of |
| | EAKKLNDAQAAA | Staphylococcal |
| | | Protein A |
| 16 | LAEAAAKEAAAKEAAAKEAAAKEAAAKAAA | Longer alpha helical |
| | | EAK Linker |
| 17 | SGSVTSTSKTTATASKTSTSTSSTS | 75 bp from A. niger |
| | | glucoamylase gene |
| 18 | AVPSGSVTSTSKTTTTASKTSTSTSSTSEF | Mixed Flexible and |
| | | Rigid |
| 19 | GGLNDIFEAQKIEWHEGKKGKGPQPQPKPQPQPQPQPKPQPKP | Mixed Flexible and |
| | EPE | Rigid |
| 20 | GPGPGHHHHHHELGGGGSGGGGSGGGGS | Mixed Flexible and |
| | | Rigid |
| 21 | GSGGGG | Flexible |
| 22 | MHWSAWWIRSNQS | Mixed Spacer |
| | | between nanoparticle |
| | | and carbon nanotube |
| | | binding protein |
| 23 | GSGGGGGSGGGGAEAAAKA | Mixed Flexible and |
| | | Rigid |
| 24 | GGGGS | Flexible |
| 25 | GGGGSGGGGS | Flexible |
| 26 | GSEAAAKEAAAKEAAAKGSGGGG | Hinge-Rigid-Flexible |
| 27 | GSGGGG | Flexible |
| 28 | MGHHHHHHHHGPLEVLFQGPVDNKFNKEQHHAFYEILHLPN | Protein A Z (HH), |
| | LNEEQRNAFIQSLKDDPSQSANLLAEAKKLNDAQAPKAAAGS | GSGGGG (SEQ ID |
| | GGGGESQVRQQFSKDIEKLLNEQVNKEMQSSNLYMSMSSWC | NO: 21), Flexible |
| | YTHSLDGAGLFLFDHAAEEYEHAKKLIIFLNENNVPVQLTSIS | Helicobacter pylori |
| | APEHKFEGLTQIFQKAYEHEQHISESINNIVDHAIKSKDHATFN | ferritin |
| | FLQWYVAEQHEEEVLFKDILDKIELIGNENHGLYLADQYVKGI | |
| | AKSRKS | |
| 29 | MGHHHHHHHHGPLEVLFQGPVDNKFNKEQQNAFYEILHLPN | Protein A Z (WT); |
| | LNEEQRNAFIQSLKDDPSQSANLLAEAKKLNDAQAPKAAAGS | GSGGGG (SEQ ID |
| | GGGGESQVRQQFSKDIEKLLNEQVNKEMQSSNLYMSMSSWC | NO: 21); Flexible |
| | YTHSLDGAGLFLFDHAAEEYEHAKKLIIFLNENNVPVQLTSIS | Helicobacter pylori |
| | APEHKFEGLTQIFQKAYEHEQHISESINNIVDHAIKSKDHATFN | ferritin |
| | FLQWYVAEQHEEEVLFKDILDKIELIGNENHGLYLADQYVKGI | |
| | AKSRKS | |
| 30 | MGHHHHHHHHGPLEVLFQGPVDNKFNKEQQNAFYEILHLPN | Protein A Z (WT); |
| | LNEEQRNAFIQSLKDDPSQSANLLAEAKKLNDAQAPKGSEAA | GSEAAAKEAAAK |
| | AKEAAAKEAAAKGSGGGGESQVRQQFSKDIEKLLNEQVNKE | EAAAKGSGGGG |
| | MQSSNLYMSMSSWCYTHSLDGAGLFLFDHAAEEYEHAKKLII | (SEQ ID NO: 26); |
| | FLNENNVPVQLTSISAPEHKFEGLTQIFQKAYEHEQHISESINNI | Mixed Helicobacter |
| | VDHAIKSKDHATFNFLQWYVAEQHEEEVLFKDILDKIELIGNE | pylori ferritin |
| | NHGLYLADQYVKGIAKSRKS | |
| 31 | MGHHHHHHHHGPLEVLFQGPVDNKFNKEQHHAFYEILHLPN | Protein A Z (HH) |
| | LNEEQRNAFIQSLKDDPSQSANLLAEAKKLNDAQAPKGSEAA | GSEAAAKEAAAK |
| | AKEAAAKEAAAKGSGGGGESQVRQQFSKDIEKLLNEQVNKE | EAAAKGSGGGG |
| | MQSSNLYMSMSSWCYTHSLDGAGLFLFDHAAEEYEHAKKLII | (SEQ ID NO: 26); |
| | FLNENNVPVQLTSISAPEHKFEGLTQIFQKAYEHEQHISESINNI | Mixed Helicobacter |
| | VDHAIKSKDHATFNFLQWYVAEQHEEEVLFKDILDKIELIGNE | pylori ferritin |
| | NHGLYLADQYVKGIAKSRKS | |
| 32 | MGHHHHHHHHGPLEVLFQGPVDNKLNKEQQNAFYEILHLPN | Protein A Z (Ca) |
| | LNEEQRKAFIQSLIDGGGDTNGNGYLDAEESANLLAEAKKLN | Zmat-8 GSGGGG |
| | DARAPKAAAGSGGGGESQVRQQFSKDIEKLLNEQVNKEMQS | (SEQ ID NO: 21); |
| | SNLYMSMSSWCYTHSLDGAGLFLFDHAAEEYEHAKKLIIFLN | Flexible Helicobacter |
| | ENNVPVQLTSISAPEHKFEGLTQIFQKAYEHEQHISESINNIVD | pylori ferritin |
| | HAIKSKDHATFNFLQWYVAEQHEEEVLFKDILDKIELIGNENH | |
| | GLYLADQYVKGIAKSRKS | |
| 33 | MGVDNKFNKEQHHAFYEILSLPNLNEEQRAAFIQSLKDDPSQS | ProA- |
| | ANLLAEAKKLNDAQAPKAAAMHWSAWWIRSNQSMATSTPK | ZHH*MHWSAWWI |
| | LVKHTLLTRFKDEITREQIDNYINDYTNLLDLIPSMKSFNWGT | RSNQS (SEQ ID |
| | DLGMESAELNRGYTHAFESTFESKSGLQEYLDSAALAAFAEG | NO: 22); Mixed |
| | FLPTLSQRLVIDYFLY | Stable Protein 1 |
| | | (SP1) |
| 34 | MGAAAMHWSAWWIRSNQSMATSTPKLVKHTLLTRFKDEITR | MHWSAWWIRSNQ |
| | EQIDNYINDYTNLLDLIPSMKSFNWGTDLGMESAELNRGYTH | S (SEQ ID NO: 22); |
| | AFESTFESKSGLQEYLDSAALAAFAEGFLPTLSQRLVIDYFLY | Mixed Stable |
| | | Protein 1 (SP1) |
| 35 | MGHHHHHHHHGPLEVLFQGPVDNKFNKEQHHAFYEILHLPN | Protein A Z (HH) |
| | LNEEQRNAFIQSLKDDPSQSANLLAEAKKLNDAQAPKVDNKF | GGGGSGGGGS |
| | NKEQHHAFYEILHLPNLNEEQRNAFIQSLKDDPSQSANLLAEA | (SEQ ID NO: 25); |
| | KKLNDAQAPKAAAGGGGSGGGGSESQVRQQFSKDIEKLLNE | Flexible Helicobacter |
| | QVNKEMQSSNLYMSMSSWCYTHSLDGAGLFLFDHAAEEYEH | pylori ferritin |
| | AKKLIIFLNENNVPVQLTSISAPEHKFEGLTQIFQKAYEHEQHIS | |
| | ESINNIVDHAIKSKDHATFNFLQWYVAEQHEEEVLFKDILDKI | |
| | ELIGNENHGLYLADQYVKGIAKSRKS | |
| 36 | MGVDNKFNKEQHHAFYEILSLPNLNEEQRAAFIQSLKDDPSQS | Protein A Z (HH*) |
| | ANLLAEAKKLNDAQAPKVDNKFNKEQHHAFYEILSLPNLNEE | GSGGGG (SEQ ID |
| | QRAAFIQSLKDDPSQSANLLAEAKKLNDAQAPKAAAGSGGG | NO: 21); Flexible |
| | GGSGGGGMLSERMLKALNDQLNRELYSAYLYFAMAAYFEDL | Pyrococcus furiosus |
| | GLEGFANWMKAQAEEEIGHALRFYNYIYDRNGRVELDEIPKP | ferritin |
| | PKEWESPLKAFEAAYEHEKFISKSIYELAALAEEEKDYSTRAF | |
| | LEWFINEQVEEEASVKKILDKLKFAKDSPQILFMLDKELSARA | |
| | PKLPGLLMQGGE | |
| 37 | GCGAGTGGCGCATCTATGGCGTTGGAAAAAATGGCGCTTG | Alpha helix linker |
| | AAAAGATGGCCCTGGAAAAGGGAAGTATGGCTTTGGAAAA | |
| | GATGGCCTTGGAGAAAATGGCACTGGAGAAGATGGCCTTA | |
| | GAGAAGATGGCATTAGAAAAAGGTAGCATGGCTCTTGAAA | |
| | AAATGGCCCTTGAGAAAATGGCGCTGGAAAAACCTTCGGG | |
| | TCGCTCAAGTGGC | |
| 38 | TCCGGCAGCGTGACCTCGACCAGCAAGACCACCGCGACTG | 75 bp from A. niger |
| | CTAGCAAGACCAGCACCAGTACGTCTTCAACCTCC | |
| 39 | GCGGTGCCGAGCGGCAGCGTGACCAGCACCAGCAAAACCA | Mixed Flexible and |
| | CAACCACCGCGAGCAAAACCAGCACCAGCACCAGCAGCAC | Rigid |
| | CAGCGAATTT | |
| 40 | GGCGGCCTGAACGATATTTTTGAAGCGCAGAAAATTGAAT | Mixed Flexible and |
| | GGCATGAAGGCAAGAAAGGCAAAGGCCCGCAGCCGCAGC | Rigid |
| | CGAAACCC | |
| 41 | GGTCCAGGTCCGGGTCATCACCATCACCACCACGAACTGG | Mixed Flexible and |
| | GTGGTGGTGGTTCTGGTGGTGGCGGTTCTGGTGGTGGTGGT | Rigid |
| | TCT | |
| 42 | GGATCTGGTGGTGGAGGG | Flexible |
| 43 | ATGCATTGGAGCGCGTGGTGGATTCGCAGCAACCAGAGC | Mixed Spacer |
| | | between nanoparticle |
| | | and carbon nanotube |
| | | binding protein |
| 44 | GGCTCTGGTGGTGGAGGGGGATCTGGTGGTGGCGGGGCGG | Mixed Flexible and |
| | AGGCGGCCGCAAAAGCG | Rigid |
| 45 | GGTGGTGGAGGGTCT | Flexible |
| 46 | GGAGGCGGTGGTTCTGGGGGCGGTGGCAGC | Flexible |
| 47 | GGCAGTGAAGCAGCCGCGAAGGAAGCCGCAGCAAAGGAG | Hinge-Rigid-Flexible |
| | GCGGCAGCCAAGGGATCTGGTGGTGGAGGG | |
| 48 | GGATCTGGCGGAGGAGGG | Flexible |
| 49 | HHHHHHHH | 8 Histidine Tag |
| 50 | GPLEVLFQGP | HRV3C Histidine |
| | | Tag Cleavage |
| | | Sequence |
| 51 | MGHHHHHHHHGPLEVLFQGP | Affinity tag |
| | | comprising a poly |
| | | histidine tag and |
| | | enterokinase cleavage |
| | | site |
| 52 | ATGGGCCATCACCATCACCACCACCATCATGGACCCCTCGA | Coding Sequence for |
| | AGTATTGTTCCAGGGTCCTGTGGATAACAAATTCAACAAAG | SEQ ID NO: 30 |
| | AACAACAAAATGCTTTCTATGAAATCTTACATTTACCTAAC | optimized for E. coli |
| | TTAAACGAAGAACAACGCAATGCGTTCATCCAAAGCCTGA | expression |
| | AAGATGACCCAAGCCAAAGCGCTAACCTTTTAGCAGAAGC | |
| | TAAAAAGCTGAATGATGCTCAAGCACCAAAAGGCAGTGAA | |
| | GCAGCCGCGAAGGAAGCCGCAGCAAAGGAGGCGGCAGCC | |
| | AAGGGATCTGGTGGTGGAGGGGAAAGCCAAGTCCGCCAGC | |
| | AATTTTCAAAAGATATCGAAAAACTGTTGAACGAGCAAGT | |
| | AAATAAAGAAATGCAGTCCAGTAACCTCTATATGTCCATGT | |
| | CCTCCTGGTGCTATACGCACAGTCTGGACGGCGCGGGTCTT | |
| | TTTCTGTTTGACCATGCCGCTGAAGAGTACGAGCACGCTAA | |
| | GAAATTGATCATCTTTCTGAATGAGAACAATGTCCCTGTTC | |
| | AATTGACCAGTATTTCCGCTCCGGAACATAAGTTTGAGGGA | |
| | TTGACGCAGATTTTTCAGAAAGCCTATGAGCATGAGCAACA | |
| | CATCTCAGAGAGCATCAACAACATCGTGGACCATGCGATC | |
| | AAGTCAAAAGACCACGCTACTTTCAACTTCCTTCAATGGTA | |
| | TGTAGCCGAACAACACGAGGAGGAGGTCCTGTTCAAAGAT | |
| | ATCTTGGATAAAATTGAACTCATTGGAAATGAGAATCATGG | |
| | CCTGTACCTCGCTGACCAGTATGTTAAAGGGATTGCTAAGA | |
| | GTCGCAAGTCA | |

### EXAMPLES

### Example 1: Systems and Methods for End to End Continuous Downstream Purification

### Capture Step

Experiments were performed using a Chinese Hamster Ovary (CHO) cell line expressing a monoclonal antibody IgG 1. Studies were conducted in 3 liter glass stirred tank reactors with a 2 liter working volume (Chemglass Life Sciences, USA) or in a 500 liter perfusion bioreactor. Cell culture was continuously harvested from days 0-14 in Tangential Flow Filtration (TFF) perfusion system using a polyethersulfone (PES) hollow fiber membrane with a 0.2 μm pore size and a 980 cm² membrane area (Repligen Corporation, USA). In the TFF system, a low shear magnetically levitating centrifugal pump was used as the recirculation device (Levitronix, Zurich, Switzerland).

### Affinity Colloidal Capture Step

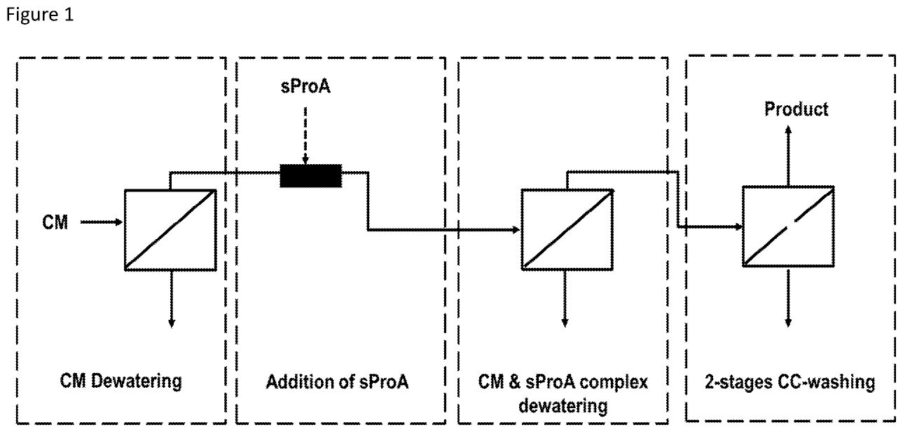

The continuous counter-current affinity colloidal capture step was comprising 4 steps as shown in FIG. 1. First, dewatering of conditioned media (CM); second, addition of soluble Protein A (sProA); third, dewatering of CM & sProA complex; and forth, 2-stages of counter-current washing.

During dewatering stage, 30 kDa Ultracel Pellicon capsule with an area of 0.5 m² was used (Cat #PCC030C05 Millipore-Sigma, USA). Two peristaltic pumps were used to control the feed and retentate flow rate (Watson-Marlow Fluid Technology Group, USA). The transmembrane pressure (TMP) was monitored by placing the PendoTech pressure sensors (Cole-Parmer, USA) on feed, retentate and permeate lines. The critical flux study was conducted using the flux-stepping procedure by Li and Zydney (Li Z, Zydney A L. Effect of zinc chloride and PEG concentrations on the critical flux during tangential flow microfiltration of BSA precipitates. Biotechnology Prog. 2017; 33 (6): 1561-1567). The TMP was evaluated as a function of time during constant flux operation, with the filtrate flux increased stepwise to determine the onset of fouling.

In order to determine the optimum conditions for the mixing of sProA and mAb, a series of studies were conducted in a batch mode using a well-behaved mAb (“mAb1”). The mAb was aliquoted into a 100 mL glass beaker and placed on a magnetic stir plate at a constant mixing rate of 300 RPM at room temperature. The sProA (Cat #10-2001-1M Repligen Corporation, USA) was added to the beaker using a programmable syringe pump at a flow rate of 0.05 mL/min. The optimum binding condition was determined by varying the molar ratios of sProA: mAb and the data were evaluated by dynamic light scattering (DLS), size exclusion chromatography (SEC), and protein A bindable.

In order to concentrate the CM and sProA mixture, a 0.1 m² Pellicon capsule with Biomax 300 kDa Membrane, C screen was used. Two peristaltic pumps were used to control the feed and retentate flow rates (Watson-Marlow Fluid Technology Group, Wilmington, MA) while TMP was monitored by placing the PendoTech pressure sensors on feed, retentate and permeate lines. The retentate from this stage was fed into a 2-stages counter-current washing step in order to further remove the impurities. The area of each filter during washing was 0.1 m², and feed and retentate fluxes of each filter was selected based on the critical flux study conducted using the flux-stepping procedure. The continuous counter-current affinity colloidal capture step is shown in FIG. 1.

### Analytical

The mAb concentration was determined by analytical ProteinA affinity chromatography using a 20×2.1 cm I.D. POROS A/20protein A column (Thermo Fisher Scientific). The antibody concentration was calculated using a standard curve that was created for the molecule of interest according to Beer's Law. The mAb purity was evaluated by size exclusion chromatography (SEC) column using a 30×7.8 cm I.D. TSKgel G2000SWxl column (Tosoh Bioscience LLC). The product purity was calculated from the SEC chromatograms as the ratio of the monomer peak area to the sum of all peak areas. The size of the mAb and sProA complex was evaluated by DLS using a Zetasizer Nano (Malvern Panalytical, UK). HCP levels were determined using enzyme-linked immunosorbent assay (ELISA) kit (Cygnus Technologies, USA). The samples were diluted using sample diluent buffer (Cygnus Technologies) until the HCP concentration in the sample was in the range of 1-100 ng/mL. The ELISA procedure followed the kit manufacturer protocol.

### CM Dewatering

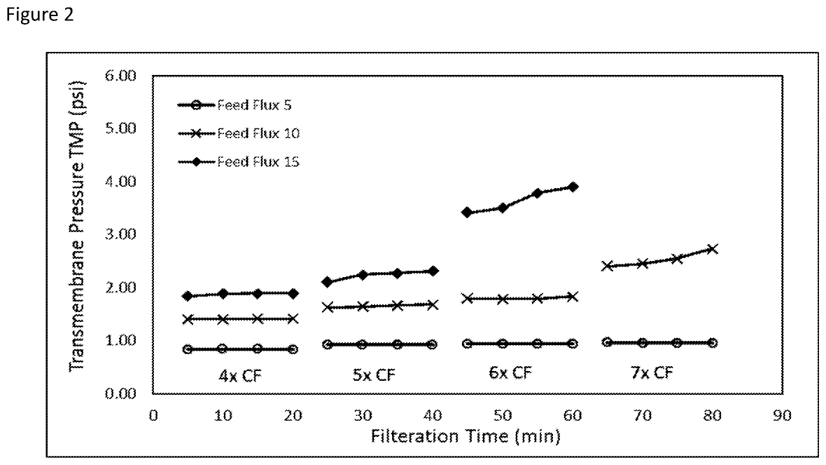

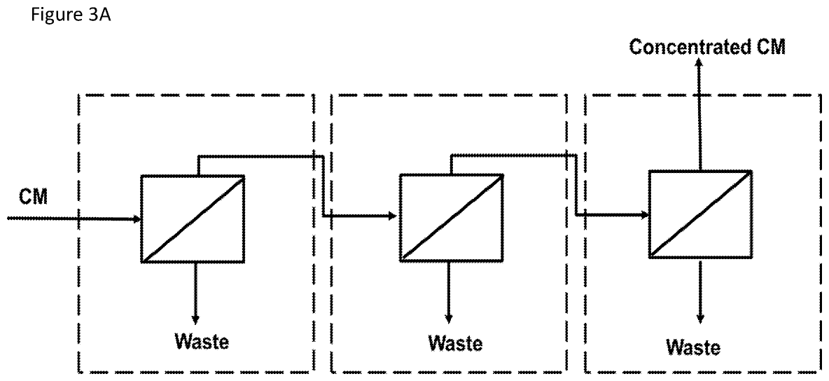

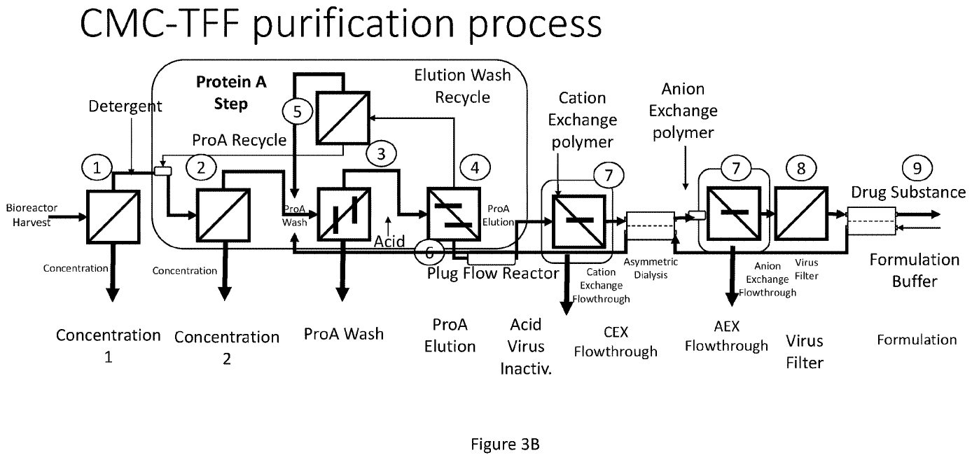

FIG. 2 shows the critical flux study for dewatering stage at a feed flux of 5, 10, and 15 LMH where the concentration of feed material (CM mAb1) was 1 mg/mL. As shown in FIG. 2, the maximum concentration factor of 7 is achieved at feed flux of 5 LMH while TMP is below 1 psi. The data generated here can be used to design the dewatering process based on the flow rates coming out of a perfusion bioreactor. The permeate flow rate can also be adjusted based on the desired final concentration factor, considering the initial concentration of CM that will be used. Based on the critical flux data generated, a 3-stage TFF for dewatering was designed where 670 L CM of mAb1 generated during days 11-13 of a 500-L perfusion bioreactor was concentrated. Two peristaltic pumps were used to control the feed and retentate fluxes on each filter as shown in FIG. 3. The average concentration of CM was 1 mg/mL and it got concentrated 8.3× during the process where the flux and pressures of each filter is described in Table 1. Close to 1-log HCPs reduction was achieved during this step with little to no measurable retention of HCPs on membrane. In addition, by reducing 0.5-1 log HCPs during this step, the final purity of the captured product can also increase mainly due to the interaction of HCPs with the complex of sProA and mAb.

TABLE 1
Flux and concentration factor during 3-stage CM dewatering

| | Filter 1 | Filter 2 | Filter 3 |
|---|---|---|---|
| Feed Flux (LMH) | 16.8 | 5.8 | 2.9 |
| Retentate Flux (LMH) | 5.8 | 2.9 | 2.1 |
| Concentration Factor | 2.9 | 2 | 1.38 |
| TMP (opsi) | 2.1 | 1.8 | 1.9 |

mAb and sProA Mixing

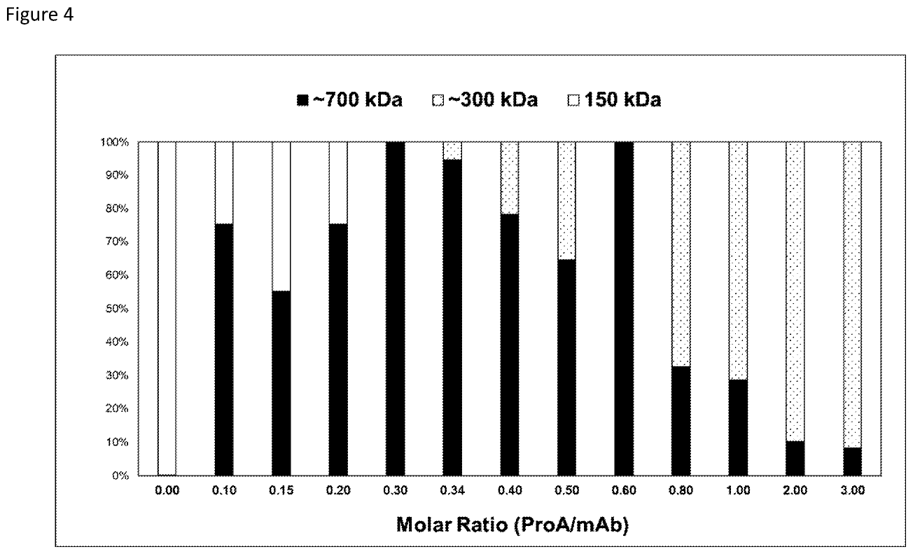

The optimum binding condition for sProA and mAb was determined by varying the molar ratios of sProA: mAb. FIG. 4 demonstrates the size of the complex that is forming at different molar ratios of sProA: mAb for mAb1. A polished mAb at a concentration of 4 mg/mL and pH of 7.4 was used for each study where 40 mg/mL of sProA at pH 7.2 was added to increase the molar ratio. The size of the complex was investigated using DLS and SEC, and based on the data a soluble complex of around 700 kDa is forming at a molar ratio of 0.3 sProA: mAb. In addition to the size of the complex, the yield of binding was also investigated using ProA bindable HPLC. For mAb1 a 100% yield was achieved at molar ratio of 0.3 and higher; where 100% yield means, all mAbs have formed a complex and there is no free mAb in the solution. Based on the data generated in this study, a molar ratio of 0.3 sProA: mAb was used for all the remaining studies reported here.

### 3-Stages Dewatering and CC-Washing

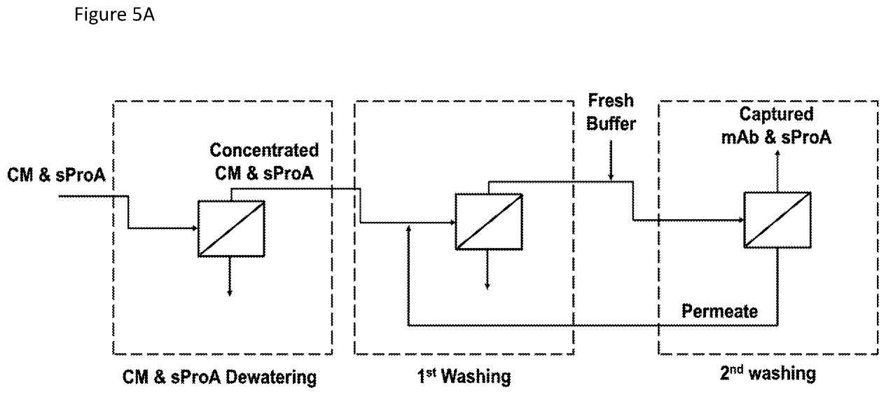

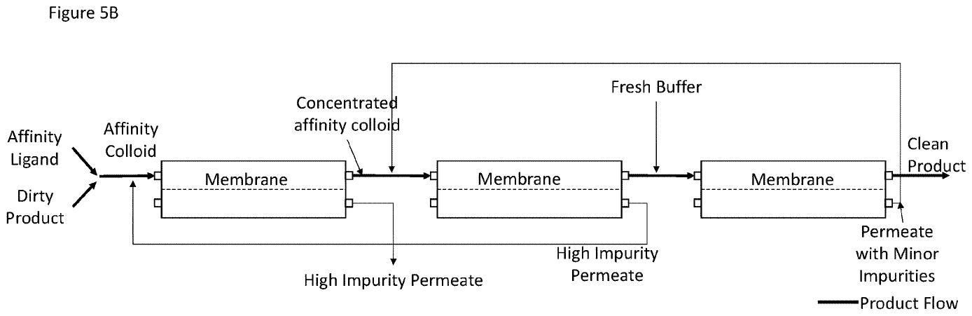

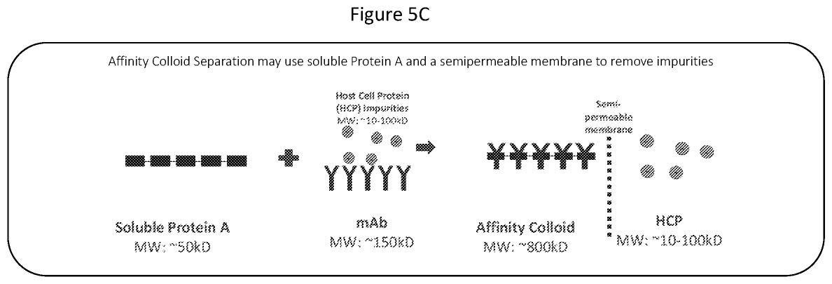

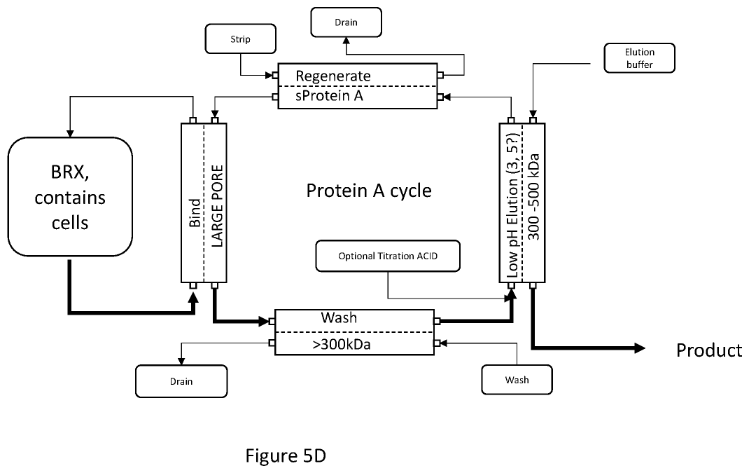

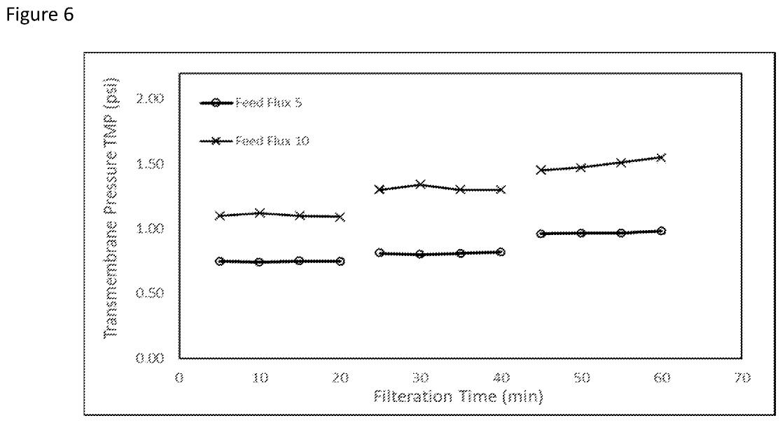

As shown in FIG. 5, a 3-stage dewatering and CC-washing was used where the mAb & sProA complex was first concentrated. Then the retentate from this stage was diluted with the permeate from the second washing stage and concentrated again on the first washing stage. Finally, the retentate from the first washing stage was diluted again using a fresh buffer of 50 mM Tris at pH 7.4, and further concentrated during the second washing stage. Critical flux studies were conducted for the washing stage to determine the optimum flow rates for this step. As shown in FIG. 6, a concentration factor of 5 can be achieved where the feed flux is 5 LMH. So, based on this data the feed and retentate flux of each stage was controlled at 5 and 1 LMH respectively in order to achieve a 5× concentration factor during each stage. At the end of the capture step, a concentration factor of 40 was achieved with the final HCPs level of 590 ppm.

These data demonstrate the HCPs level of 590 ppm with the antibody yield of 90% that operates most efficiently at high protein concentrations to reduce the amount of wash solution per kg of product. The final purity of the product is comparable with a Protein A chromatography column; with the advantage of reducing the process mass index (PMI) by a factor of at least 3. In addition, the final cost of the purification process is further reduced by replacing the expensive Protein A chromatographic resins with soluble form of Protein A, which is recycled and reused at least 10-20 times. The process is shown to be economically viable with commercially available sProA. In addition, sProA can be made by any manufacturer, including internally, thus eliminating supply chain constraints.

The extent of removal (R) for each species on a TFF is given as:

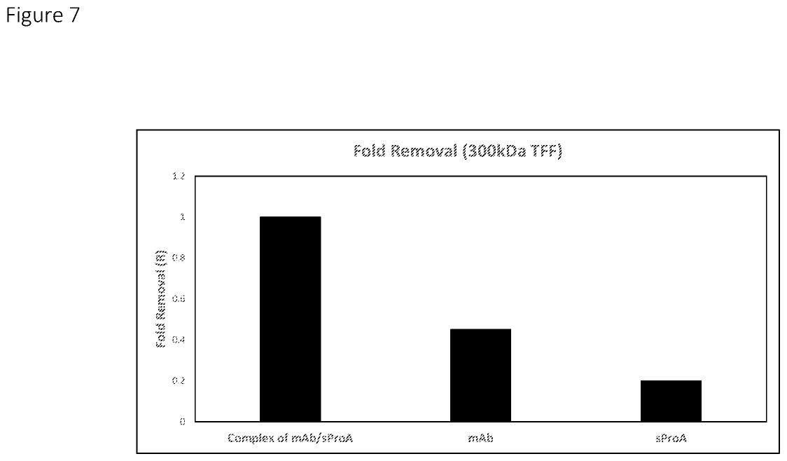

R=C₀/C, where C₀ is the concentration of species in the feed entering the filter, C is the concentration of species in the final retentate leaving the filter. FIG. 7 shows the fold removal for mAb, sProA and complex of mAb & sProA at a molar ratio of 3; where the mAb and sProA pass through the filter while 100% of complex of mAb & sProA retains to the filter.

### CEX Polishing

In order to separate sProA and mAb after capture step, a CEX polishing step was developed. The studies were conducted in batch contacting mode using 96 well-plates (cat #136101, Fisher Scientific) to determine the optimum condition for separation of mAb and sProA. UNOSphere cation exchange resin (Cat #1560111, BioRad) was prepared and washed using a buffer comprising of 50 mM Glycine at pH 3, then it was settled and centrifuged at 1000×g for 10 min in order to determine the volumetric concentration. The complex of mAb & sProA from capture step was titrated to pH 3 using Glycine/HCl, and it got mixed with 20% resin at 20 mg/ml binding capacity. After incubating the sample for 5 minutes, it was centrifuged at 1000×g for 10 min to collect the filtrate containing the unbound proteins. During the next step, in order to elute sProA, first elution buffer was mixed with samples and then it was centrifuged to collect the filtrate containing sProA. During the last step, the second elution buffer was mixed with the samples, and centrifuged to collect the mAb as the filtrate in this step. The volume of the buffer during both elution steps were 20× the volume of the settled resin, and the incubation time was 10 and 20 minutes for the first and second elution step before centrifuging the samples.

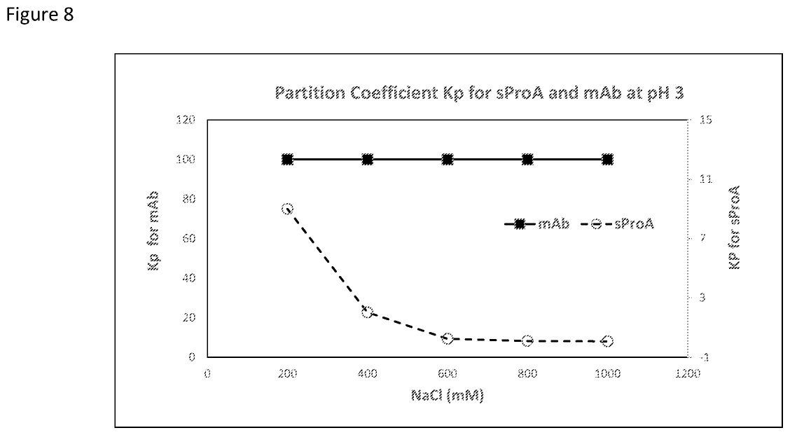

FIG. 8 shows the data generated during the first elution of CEX polishing step where the partition coefficient of sProA and mAb1 is measured at pH 3 and different concentration of NaCl. Based on the data 0.8 M NaCl is able to elute 91% of sProA at pH 3 without eluting any mAb in this step. During the second elution step, 50 mM Tris and 1 M NaCl at pH 7.8 was used which results in elution of 70% mAb.

### AEX Polishing Materials and Methods

A synthetic feed (mimicking neutralized protein A elution pool) containing monoclonal antibody mAb1 and impurities viz. HCP was adjusted to pH 8.5 and salinity of 10 mM NaCl (conductivity, 2.5±0.3 mS/cm). The synthetic feed was prepared by spiking the mAb with HCPs enriched from condition media (mAb1). The enriched HCPs were prepared by loading the mAb1 conditioned media (adjusted to pH 8.5, conductivity 2.5 mS/cm) onto the Capto Q column (20 cm bed height, 1.1 cm diameter) pre-equilibrated with buffer A, 10 mM Tris pH 8.5, 10 mM NaCl. Following a post-load column wash with buffer A, the bound HCPs were recovered by a step elution with 10 mM Tris pH 8.5, 1 M NaCl. The eluted HCP pool was treated with 10 μL of Benzonase endonuclease (EMD Merck, Germany) for 2 hours to digest host cell DNA and RNA. The resulting HCP pool was buffer exchanged by dialysis against buffer A using Slide-A-Lyzer™ Dialysis Flasks, 3.5K MWCO (Thermo Scientific, USA). The mAb concentrations were determined using Solo-VPE (Repligen, USA). Chromatographic separations were performed using Fractogel TMAE (M) anion exchange resin (EMD Merck, Germany) with a 40-90 um particle size range as stated by the manufacturer. The resin was washed and resuspended in binding buffer, 10 mM Tris HCl pH 8.5, 10 mM NaCl. The adsorbent and binding buffer conditions were empirical and described elsewhere (Kelley et al., 2008). The resin slurry concentration (% v/v) was determined from the settled resin volume following centrifugation at 1000×g for 5 mins.

### Batch Binding Experiments

The screening of adsorbents and optimal binding conditions for impurity removal was done using batch binding experiments. This approach allows easier and faster screening while maintaining a functional resemblance to the intended continuous operation. The anion exchange resin, e.g., Fractogel TMAE, was pre-equilibrated and resuspended in buffer A (as stated above) to make a 50% (v/v) resin slurry. A required amount of resin slurry was pipetted into 1.5 mL low protein-binding tubes (Cat. #90411, Thermo Scientific) containing a mAb pool with or without impurities to yield a final resin suspension of 1% or 5%. The mAb pools were pre-conditioned with buffer A. The resultant mixture was incubated for 20 minutes on a rotatory mixer at room temperature (22±2° C.). After incubation, the resin containing samples were filtered through 0.45 μm spin filters to recover the polished mAb. The mAb concentrations were measured on Solo-VPE (Repligen, USA). The HCP and host-cell (CHO) DNA in the unpolished and polished mAb were quantified using in-house assays for HCP ELISA and real-time PCR, respectively.

It should be noted that some of the screening studies were performed in 1 mL 0.45 um 96-well filter plates (Cat #8129, Pall Corporation, USA). The operating conditions (pH and conductivity) were selected based on the weak-partitioning mode (WPC) of operation (Citation). In WPC, differential interactions of the product of interest and impurities with the chromatography resin are represented by partition coefficient, Kp. For a protein (mAb or impurity), Kp is the ratio of the concentration of the solute bound to the resin divided by the concentration in solution at equilibrium (cite).

$${K}_{p} = \frac{[ bound ]}{[ free ]}$$

### Continuous Single-Pass TFF Anion Exchange Chromatography

As shown in FIG. 9, the continuous single-pass TFF anion exchange chromatography system comprises a hollow fiber module with a surface area of 980 cm² (Cat #S04-P20U-10-N, Repligen, USA) and peristaltic pumps. The feed and permeate flow rates were controlled using peristaltic pumps. This arrangement allowed for the regulation of permeate flux given the high normalized water-permeability (400 LMH/psi) of the hollow fiber modules used in this study. The resin slurry (40% v/v) was pre-equilibrated with binding buffer, 10 mM Tris HCl pH 8.5, 10 mM NaCl. The slurry was batch-wise mixed with feed (10 g/L mAb and impurities) manually to yield a load material with desired final resin slurry concentration of 1% to 5%. This mixture was incubated for 5 minutes and was applied to the hollow fiber module at a feed flux of 50 LMH using the feed pump, P1. The flow rates between pumps P1 and P2 were orchestrated to attain the desired permeate flux and slurry concentration factor along the hollow fiber membrane, where P1>P2 and the slurry concentration factor is the ratio between the feed and the retentate flowrates. The choice of slurry concentration factor was dependent on the percentage of slurry in the load material and intended product recovery in a single-pass operation. The chosen slurry concentration factors were targeted for 20× and 5× for initial resin slurry of 1% and 5%, respectively (Table 2), which resulted in a final slurry concentration of nominally 20% and 25% after the SPTFF. The purified mAb product was continuously collected in the permeate, while the impurity-laden concentrated resin was recovered from the retentate.

TABLE 2
Comparison of HCP reduction in the synthetic feed
at different monoclonal antibody concentrations.

| | Resin | Slurry | | Polished | |
|---|---|---|---|---|---|
| | slurry | concentration | Feed | product, | HCP, |
| Feed | in load | factor | HCP | HCP | Log |
| mAb (g/L) | material | in retentate | (ppm) | (ppm) | Reduction |
| 10 mg/mL | 5% | 5x | 600 | 7 | 1.9 |
| | 1% | 20x | 600 | 8 | 1.9 |
| 85 mg/mL* | 1% | 20x | 426 | 76 | 0.75 |

*No Benzonase treatment
Water reclamation and PMI

### Slurry Concentration Factor

Percent product recovery or step yield in a single-pass operation was directly proportional to the slurry concentration factor along with the hollow fiber module. Slurry concentration factors successfully evaluated for 5% resin slurry were 5×, 10×, 15×; for concentration factors beyond 15, a sharp increase in transmembrane pressure up to 25 psi was likely due to occlusion of the hollow fibers.

### Batch Binding Experiments

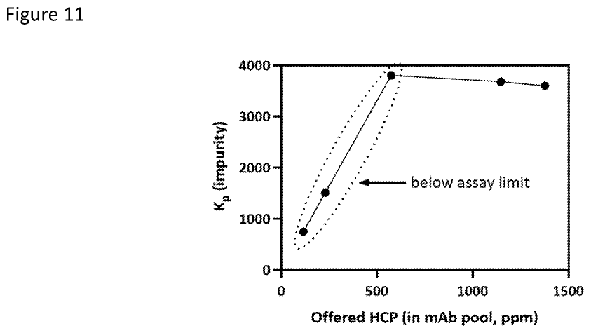

FIG. 11 shows the calculated ‘Kp impurity’ values obtained at various HCP concentrations while keeping the mAb concentration constant at 10 g/L with 5% Fractogel TMAE resin. In the case of the HCP challenge of 600 PPM or less, the residual HCP levels in polished mAb material were below the assay's detection limit. At higher (>600 ppm) HCP challenges, the 2.3 log-reduction in HCP was observed with Kp impurity values for the HCPs were >3500. Similarly, partition coefficients for the purified mAb product were determined and were found to be <1.0 (data not shown) at the salinity of 10 mM (NaCl). From the principles of WPC, the lower product Kp values are preferred as it reduces the potential for product loss, especially at higher mAb loadings, and allows maximum utilization of the effective binding capacity of the adsorbent for HCP and DNA removal.

### Continuous Single-Pass TFF Anion Exchange Chromatography

The functional similarity of the batch binding studies to the TFF system simplified the switch to the TFF mode of operation while leveraging the results from the batch binding. Table 1 shows the HCP reductions observed while operating in the TFF mode. For both 1% and 5% resin slurry at 10 mg/mL mAb feed, the 1.9 log-reduction in HCP was observed that was comparable to batch binding results. The choice 1% resin slurry afforded product recovery of 91% compared to 74% product recovery observed with 5% slurry in TFF mode. It should be noted that the product discussed here is a consequence of the slurry concentration factor and not the product Kp (Kp<1). Interestingly, a marked decrease in HCP log-reduction at 85 mg/mL mAb feed was observed.

In-process water reclamation from the purified product in permeate would offset the PMI of this step. As shown in FIG. 9, mAb feed, 35 g/L was concentrated to 80 g/L using 30 kDa Single-Pass TFF module to recover 56% of water feed with no detectable HCP breakthrough in the reclaimed water (Table 3).

TABLE 3
Water reclamation and host cell protein breakthrough
in reclaimed water from mAb feed

| mAb feed | mAb, Post UF | Feed HCP | HCP, Reclaimed |
|---|---|---|---|
| (mg/mL) | (mg/mL) | (ng/mL) | water (ng/mL) |
| 35 | 80 | 1287 | <LOQ (<30) |

Additional reclamation strategies include but are not limited to: 10 g/L mAb containing permeate concentrated to 150 g/L would recover 93% of the water used in load material; 25 g/L mAb containing permeate concentrated to 150 g/L would recover 83% of the water used in load material; 50 g/L mAb containing permeate concentrated to 150 g/L would recover 66% of the water used in load material; and 100 g/L mAb containing permeate concentrated to 150 g/L would recover 33% of the water used in load material.

As outlined above, 1% and 5% resin slurry in load material was evaluated for slurry concentration factors 20 and 5, respectively. HCP loading on resin for 1% and 5% was 0.6 mg/mL of resin and 0.12 mg/mL of resin respectively. A 2-log HCP removal was observed at feed mAb concentration of 10 g/L.

### Example 2: Design and E. coli Expression of Ferritin Nanoparticle

### Particle Design

A protein nanoparticle could comprise of a monomer that self-assembles into a 24-mer particle via a self-assembling domain derived from Helicobacter pylori ferritin (Hpftn). An n-terminally modified version, Bf-HpFtn, was utilized. The design builds on the n-terminus of Bf-HpFtn by adding a linker domain, an antibody binding domain and an affinity tag. Optimization of particle design occurred primarily by varying linker region resulting in the choice of nanoparticle 384.

The self-assembly domain (Bf-HpFtn) of nanoparticle 384 comprises the peptide according to SEQ ID NO: 1. This is connected to the antibody binding domain by a linker, and one incarnation of this linker comprises SEQ ID NO: 26. The antibody binding domain (Z-domain) comprises SEQ ID NO: 4. The affinity tag comprises poly histidine tag and enterokinase cleavage site described in SEQ ID NO: 50. The entirety of the protein monomer composed of the above parts in nanoparticle 384 is thus SEQ ID NO: 30.

### Construct Design and Generation

The protein sequence of the protein monomer was back translated to a nucleotide sequence in Geneious Prime® software selecting for E. coli optimization with randomized codon usage minus rare codons. The resulting sequence of the designed coding gene can be found in SEQ ID NO: 52. The synthesized nucleotide was ordered from a commercial vendor with vector overlaps designed for cloning into an in-house vector derived from pET28 but with a B-lactamase resistance gene substituted into the vector. The insert was cloned by overlap assembly using a commercial Gibson reaction based cloning kit and transformation into an E. coli cloning strain. The resulting plasmid from single colony isolate was confirmed by Sanger sequencing then the plasmid was transformed into E coli BL21 (De3) for expression to form strain BI_NP0384.

### Expression of Nanoparticle

The protein from strain BI_NP0384, was produced by via fed-batch growth and IPTG induction of the expression strain. The growth medium comprises 20.3 g/kg Yeast extract, 10.1 g/kg sodium sulfate and 7 g/kg dibasic potassium phosphate and supplemented post autoclave to 100 μg/mL carbenicillin. The strain was grown at 37 C in shake flask overnight then inoculated into 30 L of the same medium at 37 C in a bioreactor, and feed cycle was started after initial oxygen rebound. Feeds comprise 550 g/kg glucose supplemented with 5.4 g/kg magnesium sulfate and 333 g/kg yeast extract. Induction was begun at optical density 550 nm of 80 by addition of IPTG to 0.3 mM final, with all available precautions to maintain oxygen supply. The culture was cooled prior to harvest after 3 hours of induction. Cell paste was harvested by centrifugation and stored at −80 C.

### Nanoparticle Purification for Insoluble Fraction (Inclusion Bodies)

For protein purification, cells were suspended to six volumes (ml/g cell paste) in phosphate buffered saline pH 7.4 (PBS). After complete resuspension by agitation, the cell suspension was kept stirring by magnetic stir bar, Proteinase inhibitor (2.3 tablets/L of Roche Complete 04693132001) and DNase (Pierce Universal Nuclease 2656968, 100 uL/L; DNase I Roche 10104159001, 33 mg/L) were added. The cell suspension was stirred then lysozyme (Thermo 89833) was added to 40 mg/L and cells were stirred for ˜40 minutes at room temp. The cell suspension was chilled on ice then cells were passed through an LM20 Microfluidizer at 12,000 psi two times while keeping reaction chamber and cooling loop immersed in an ice water bath. Lysed cells were centrifuged at 3000×g in 1 L centrifuge bottles and the supernatant was decanted. Inclusion body pellets were then suspended in 0.5× B-PER II (Thermo 78260) extraction reagent in PBS at a rate of 5 mL/g pellet with additional Dnase I 33 mg/L added. Suspension was accomplished by agitation, then the suspension was centrifuged in 500 mL centrifuge bottles at 10,000×g for 20 min. Supernatants were decanted and pellets were then washed in PBS (5 mL/g pellet). The suspension was centrifuged at 10,000×g for 20 minutes and decanted.

### Urea Solubilization of Inclusion Bodies

Washed inclusion pellets were then suspended via agitation in 5 ml/g pellet in PBS until fully suspended then three volumes of 8 M/kg Urea, 50 mM Tris pH 7.8 was added to stirring solution of inclusion suspension (6 M Urea/kg final). The solution was kept at 4° C. overnight then warmed to RT and centrifuged at 15,000×g for 20 minutes. The supernatant containing solubilized protein monomer was further purified with immobilized metal affinity chromatography (IMAC).

### Immobilized Metal Affinity Chromatography (IMAC) Purification (2.1×19 cm)

The supernatant containing urea solubilized protein was filtered through a 0.45 μm filter and loaded onto a 77 mL Ni-NTA column (2.1×19 cm) previously equilibrated with 5 mM Imidazole in 50 mM Tris-HCl, 6M Urea, pH 7.8 for 5 column volumes (CVs) at a flow rate of 15.2 mL/min. Post sample loading, the column was washed with 5 mM Imidazole in 50 mM Tris-HCl, 6M Urea, pH 7.8 for CVs to remove weakly bound impurities. Following column wash, the bound protein was eluted with a step gradient of 500 mM imidazole in 50 mM Tris-HCl, 6M Urea, pH 7.8. Post elution, the column was cleaned with 0.5 M sodium hydroxide for 5 CVs followed by 5 CVs of deionized (DI) water and 20% ethanol storage solution. To avoid loss of chelated Nickel on the Ni-NTA resin, the column was recharged with 3 CVs of 0.1M NiSO4 before each run, followed by 5 CV washes of DI water and equilibration buffer. The eluted protein was collected in the AKTA Avant fractions collector and held at 6° C. until refolding step.

### Dialysis for Urea Removal and Refolding

Batch dialysis was carried out principally to remove urea, enabling the simultaneous refolding and self-assembly of the nanoparticle alongside conditioning into a favorable buffer. IMAC eluate equilibrated to ambient temperature was loaded in A 10 kDa MWCO Slide-A-Lyzer™ Dialysis Cassette (Thermo Fisher) and stirred for one wash, overnight in 200 diavolumes (DV) of 50 mM Tris buffer at pH 7.8 and filtered with 0.22 μm filter, then stored at 4° C.

### Preparative SEC

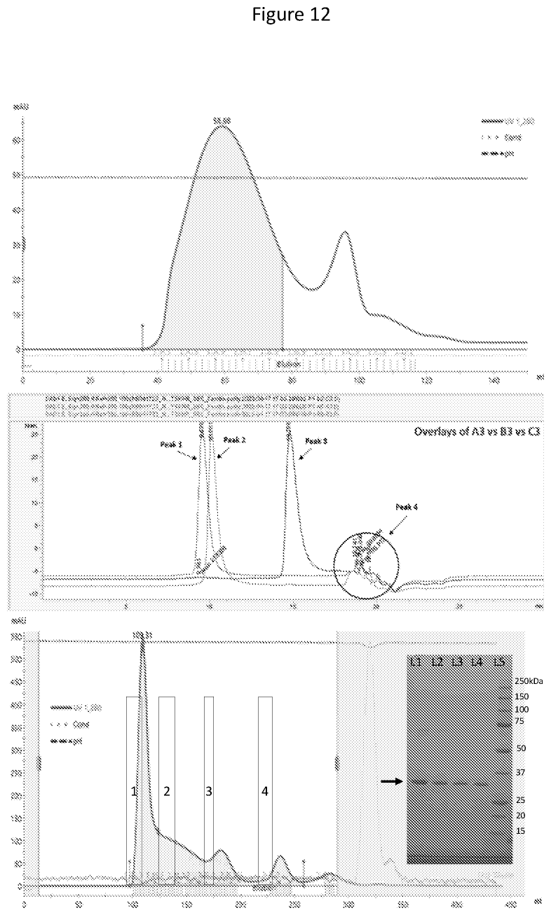

3 mL of 0.22 μm syringe filtered, dialyzed nanoparticle 384 (Ferritin) was loaded, using an AKTA® Pure (Cytiva), onto with a HiPrep™ 16/60 Sephacryl® S-500 HR (120.6 mL column volume (CV); Cytiva) size exclusion chromatography column equilibrated with 50 mM Tris pH 7.8. The column was isocratically eluted at 0.5 ml/min (15 cm/h) with equilibration buffer for at least 2CV collecting fractions A₂₈₀≥5 mAU in a 96-well deep-well plate with a maximum fraction volume of 1 mL. The column was cleaned with 0.5CV of 0.5M NaOH, 1.5CV of Equilibration buffer, 1CV of water, and 1.2CV of 20% ethanol at the same flowrate. All buffers were 0.22 μm filtered before use. Fractions were analyzed for protein concentration (A280, EC1%=7.7) on a Stunner (Unchained Labs, Pleasanton CA) and for purity with the TSKgel SuperSW mAb method described herein. Fractions were pooled to include only those fractions with peak 1 and peak 2 as in FIGS. 12A-12B. Peak 3 contains smaller fragments suspected to be incompletely assembled ferritin species and so were excluded.

### Nanoparticle Binding, Disassociation, and Characterization

When preparing samples containing both the nanoparticle and antibody, precipitation was immediately visible. To resolve this, the precipitated sample containing excess antibody to nanoparticle was added to multiple salt solutions and resolubilization was assessed visually by appearance and through absorbance readings at 600 nm as a measure of light scattering by particles in solution. In addition, the nanoparticle alone and the antibody alone were also added to these salts as a control and to determine if the salts influenced the precipitation of either of these components on their own. The precipitated sample of antibody and nanoparticle was found to have the best resolubilization by being placed in a solution of 150 mM NaCl. In addition, this condition did not result in the precipitation of either of the components by themselves. However, adding salt to the already precipitated sample did not result in complete resolubilization of the precipitated sample.

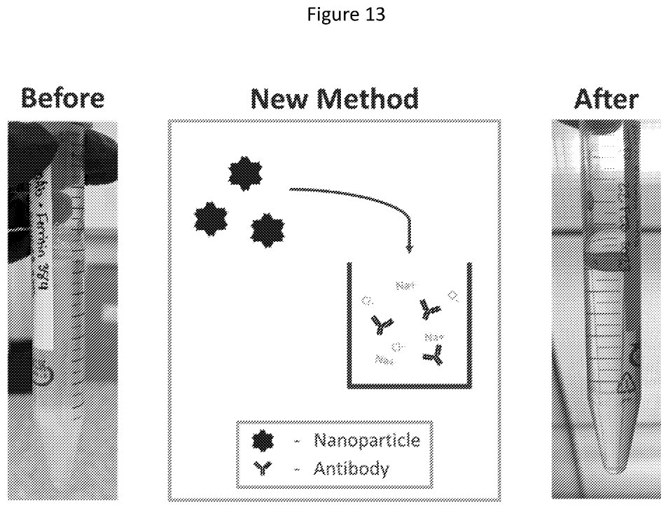

Based on the hypothesis that the precipitation interaction may be difficult to reverse completely, but might be prevented, salt was added to the antibody before adding the nanoparticle. A method of creating the bound nanoparticle and antibody samples was devised where a stock solution of 5M NaCl was added to a solution containing antibody and then nanoparticle was added to this solution. The amount of NaCl stock solution that is added to the antibody was calculated such that the final concentration of the solution was equal to 150 mM NaCl. When this method was tested, it resulted in a clear sample with no visible precipitation, as can be seen in FIG. 13.

### IgG Sepharose 6 Affinity Column (“Reverse Protein a, mAb Column”)

A set of samples containing nanoparticle were loaded onto a 3.9 mL Tricorn 5/20 column (Cytiva, Marlborough, MA) packed to a bed height of 19.7 cm with IgG Sepharose 6 Fast Flow affinity resin (Cytiva) connected to an AKTA® Pure 25 (Cytiva) FPLC system. For all runs the method followed the sequence followed the Table 4 at a set linear velocity of 300 cm/h.

TABLE 4
Chromatogram method for IgG 6 Sepharose
Fast Flow Affinity Chromatography

| | Duration | | Fraction |
|---|---|---|---|
| | (CV) | Buffer | volume (CV¹) |
| CIP² (2x | 2 | 50 mM Tris, 150 mM NaCl, | — |
| repeat) | | 0.05% Tween20, pH 7.4 | |
| | 2 | 100 mM Glycine, pH 3.6 | — |
| Equilibration | 5 | 50 mM Tris, 150 mM NaCl, | — |
| | | 0.05% Tween20, pH 7.4 | |
| Load | Variable | Sample Buffer | 0.5 |
| Wash | 10 | 50 mM Tris, 150 mM NaCl, | 0.5 |
| | | 0.05% Tween20, pH 7.4 | |
| Elution | 4 | 100 mM Glycine, pH 3.6 | 0.25 |
| Equilibration | 5 | 50 mM Tris, 150 mM NaCl, | — |
| | | 0.05% Tween20, pH 7.4 | |
| Storage | 5 | 50 mM Tris, 150 mM NaCl, | — |
| | | 0.05% Tween20, pH 7.4 + | |
| | | 20% (v/v) Ethanol | |

¹Column Volume. ²Clean-in-place sanitization method.

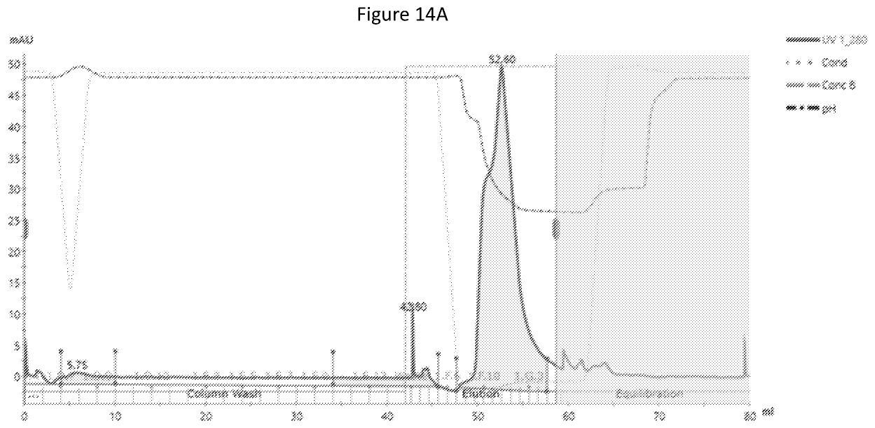

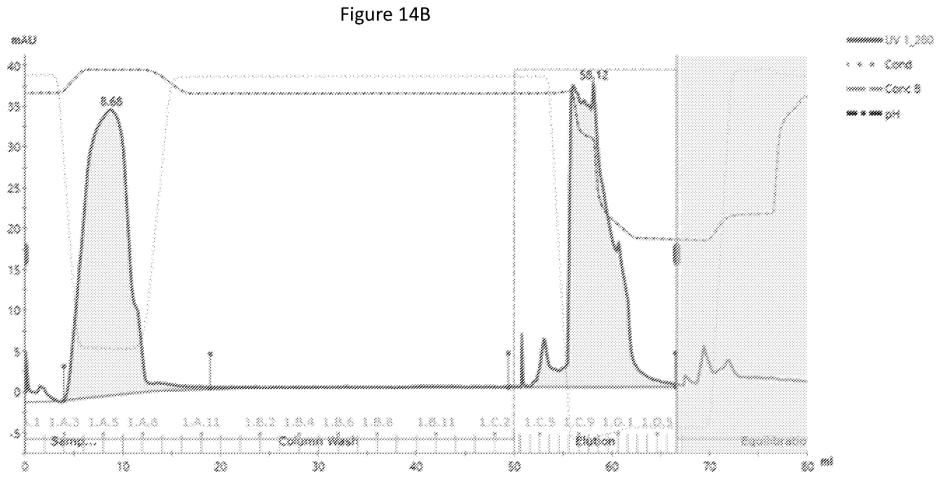

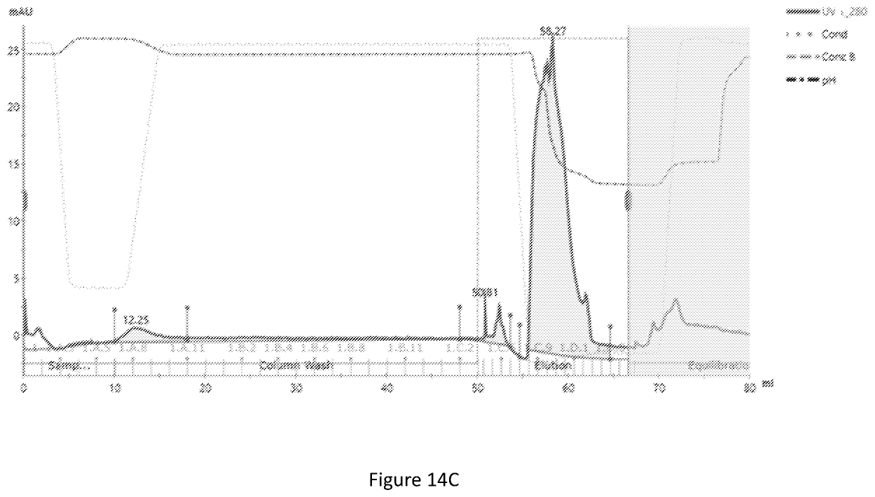

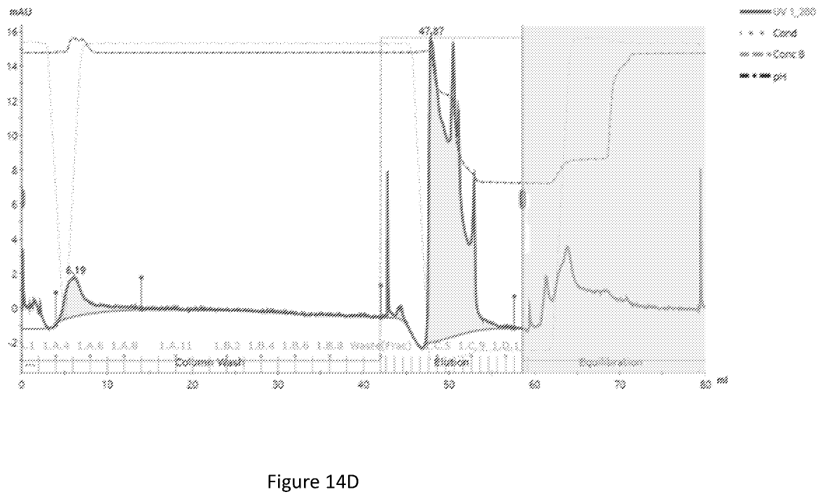

Four chromatography runs were carried out as in Table 5 and results are presented in FIGS. 14A-14D. An engineering run (cycle 0) was first carried out to confirming the binding competence of the resin (i.e. that the resin can indeed bind protein A). Recombinant native protein A ligand from S. aureus (rSPA, Repligen, Waltham, MA) diluted to pH 7.4 with 50 mM tris, pH 7.4 was loaded and, despite 25% losses on the column by mass balance, all the chromatogram area and 75% of the quantified mass is attributed to the eluate suggesting the column was functional. When loaded at less than half the mass of the soluble protein A load (cycle 1), from chromatogram peak area, roughly half of the purified nanoparticle load bound and appeared in the eluate while half appeared in the flowthrough. Cycle 1b shows that population in the flowthrough is also competent to bind to the column suggesting it was overloaded in cycle 1 and so had a lower capacity than soluble protein A, likely due to its much larger size and so lack of access to some ligands. Cycle 2 showed that when the eluate from cycle 1 is titrated to neutral pH with 0.5M Tris, pH 11 and loaded again, it can bind again. Taken together, these data demonstrate that the nanoparticle can bind the mAb, disassociate from mAb (enabling elution), and be regenerated by pH titration to bind the mAb at least once more.

TABLE 5
Affinity chromatography experiments and performance (recovery) metrics.

| | | | | | FT | | | | ELUATE | | | |
|---|---|---|---|---|---|---|---|---|---|---|---|---|
| | | | | Pooled | RECOVERY | | | Pooled | RECOVERY | | | Acc. Mass |
| | Load | | Column | FT | % | | | eluate | % | | | Loss on |
| | mass | Load | load | Mass | Peak | % | Eluate | mass | Peak | % | Quant. | Column |
| CYCLE | (mg) | pH | (mg/mL | (mg) | Area | Mass | pH | (mg) | Area | Mass | method | (%) |
| 0 | 7.6 | 7.4 | 1.90 | n.r.¹ | 0% | n.r.¹ | n.r.¹ | 1.2 | 100% | 76% | Solo VPE | 24% |
| 1 | 3.3 | 7.4 | 0.83 | 1.2 | 51% | 37% | 4.2 | 1.2 | 49% | 36% | Solo VPE | 27% |
| 1B | 1.2 | 7.4 | 0.29 | n.r.¹ | 4% | n.r.¹ | n.r.¹ | n.r.¹ | 96% | n.r.¹ | Solo VPE | n.d.² |
| N | 0.28 | 7.8 | 0.07 | n.r.¹ | 10% | n.r.¹ | 4.2 | 0.45 | 91% | 36% | Stunner | 64% |

¹not recorded,
²not determined

### Combined SEC and SDS-PAGE for Complex Formation and Disassembly

To demonstrate the formation of a complex between antibody and nanoparticle, an experiment using both SEC and SDS-PAGE was performed. First, 4.2 mL of antibody at a concentration of 9.6 mg/mL in buffer containing 50 mM Tris and 215 mM NaCl at pH 7.8 was stirred while 1.8 mL of nanoparticle in 50 mM tris at pH 7.8 and a concentration of 5.1 mg/ml was continuously added at a rate of 0.1 mL/min. The resulting mixture contained antibody at 6.7 mg/mL and nanoparticle at 1.5 mg/mL in buffer containing 50 mM tris and 150 mM NaCl at pH 7.8.

This material was then analyzed using a HiPrep 16/60 Sephacryl S-500 HR column (Cytiva). The column was connected to an AKTA® pure and every step was run at a flow rate of 14.9 cm/hr. From initial storage in 20% ethanol in water, the column was washed with 1 CV of water and then equilibrated with 2 CVs of 50 mM tris at pH 7.8. The antibody and nanoparticle mixture was loaded at a volume of 3 mL, or 2.5% of the column volume, using a 50 mL Superloop by Cytiva, and 2 CV of the same equilibration buffer was run through the column to elute the mixture. The elution peaks, measured at an absorbance of 280 nm, were fractionated in 2 mL increments. The column was then sanitized with 0.75 CVs of 0.5M NaOH, washed with 1 CV of water, and then placed back into the 20% ethanol in water storage buffer by applying 1.2 CVs. The same method was repeated two more times with a 3 mL load of antibody alone at 17.4 mg/mL and with a 3 mL load of nanoparticle alone at a concentration of 5.0 mg/mL.

The fractions from the antibody and nanoparticle mixture run were then analyzed using SDS-PAGE according to the protocol described below.

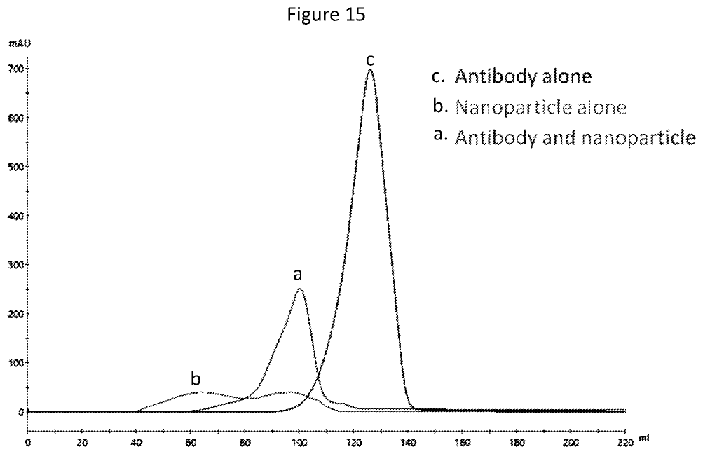

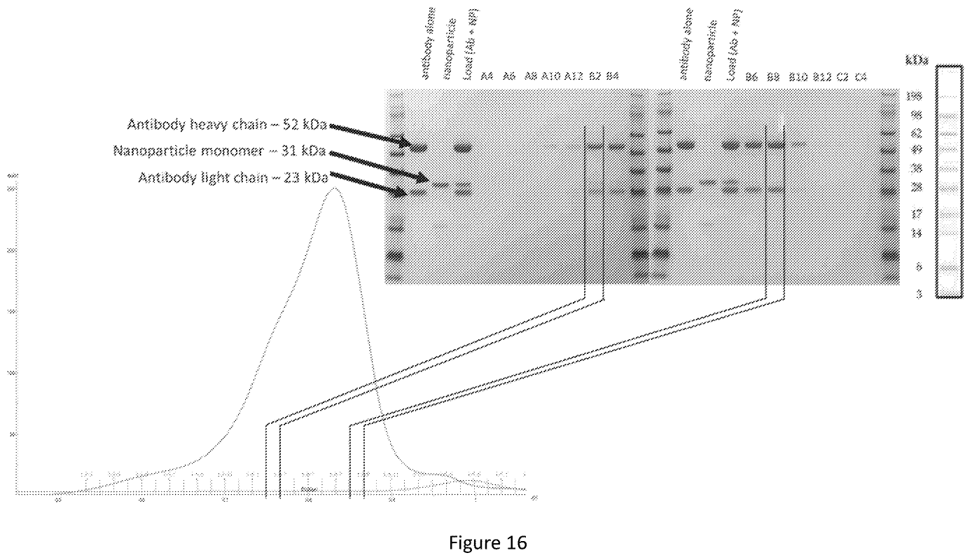

The SDS-PAGE results (FIG. 16) show that the front of the peak contains nanoparticle and antibody, while the back of the peak only includes antibody. This can be seen through comparison of fractions B2 and B8 on the gel, where the line that shows nanoparticle monomer is visible in B2 and not in B8. In addition, evaluation of FIG. 15 indicates that the antibody alone does not elute before ˜100 mL of elution volume corresponding to fraction B6 on the gel, but when antibody and nanoparticle are combined, antibody can be seen as early as 80 mL. Because the antibody is appearing at an earlier elution volume than when it is alone, which indicates a larger size, we conclude that antibody is binding to the nanoparticle.

To demonstrate the low pH disassociation of antibody and nanoparticle, this use of SEC and SDS-PAGE in conjunction with one another was repeated, but at a low pH. Just as performed above, a mixture containing antibody at 6.7 mg/mL and nanoparticle at 1.5 mg/mL in buffer containing 50 mM tris and 150 mM NaCl at pH 7.8 was created. Then, this solution was titrated down to pH 3.5 using 1M Glycine at pH 3.5. 1 mL of low pH buffer was added for every 2 mL of original mixture, diluting the sample to an antibody concentration of 4.5 mg/mL and a nanoparticle concentration of 1.0 mg/mL, all at pH 3.5.

For the SEC procedure, the same HiPrep 16/60 Sephacryl S-500 HR column (Cytiva) was connected to an AKTA® pure, and the same running method was used with two main differences. First, the equilibrium buffer for this run was 100 mM Glycine pH 3.5 such that the column environment could be kept in a low pH condition. Second, an additional wash step with 0.5 CVs of water was added before sanitization with 0.5 M NaOH to prevent any interactions between the acid and base. The mixture of antibody and nanoparticle was loaded at 5.5 mL, or 4.5% of the column volume, which is increased to account for the dilution from the titration procedure. This same procedure was repeated for the controls, where 5.5 mL of the antibody alone was injected at 4.5 mg/mL and 5.5 mL of nanoparticle alone was injected at 1.0 mg/mL.

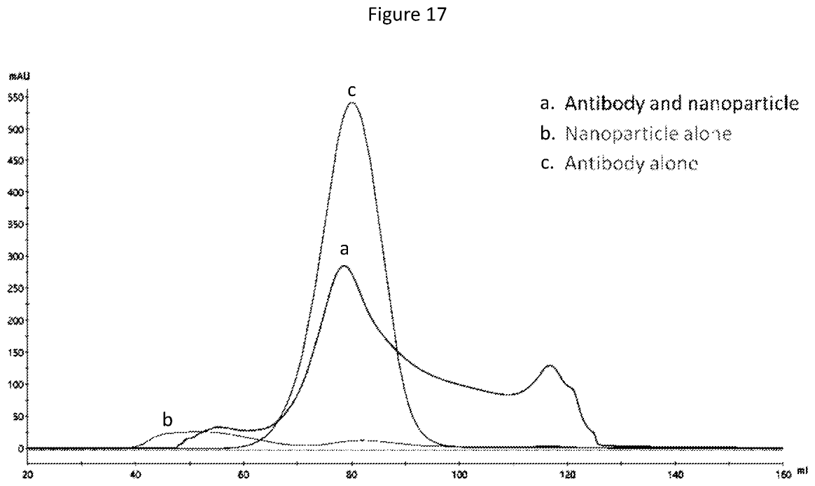

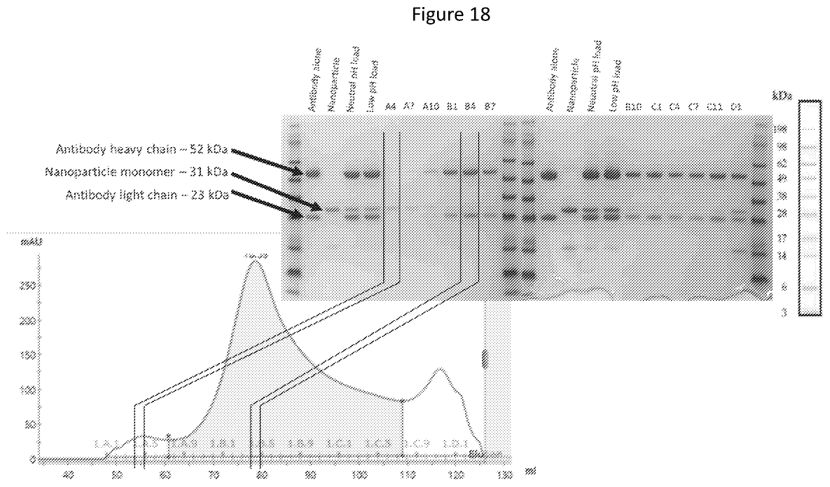

The fractions for the low pH SEC run with the antibody and nanoparticle mixture were then run on a SDS-PAGE gel according to the method presented below. The antibody and nanoparticle mixture resulted in two peaks (FIGS. 17 and 18); the first is entirely nanoparticle and elutes where the nanoparticle alone peak would typically elute, and the second is an antibody peak that elutes where the antibody alone peak would typically elute. Unlike the neutral pH experiment, where the antibody elutes earlier (due to larger size) than it typically would when injected alone, the antibody here does not show an increase in size according to the time it elutes from the SEC column and instead elutes at the same residence time as when there is no nanoparticle mixed in the load. Therefore, this demonstrates that at a low pH, the antibody and the nanoparticle are completely disassociated.

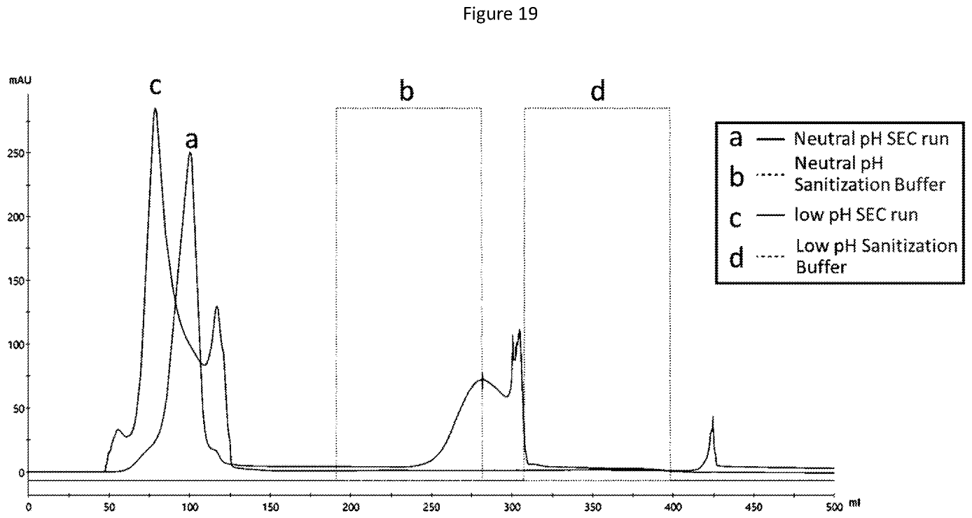

In FIG. 19, the SEC traces of the antibody and nanoparticle mixture load at neutral pH and low pH are compared. The sanitization peaks are located at different points on the x-axis because of the extra water wash in the low pH method. However, it is immediately apparent that the peak following sanitization is much larger in the neutral pH run than in the low pH run. This indicates that more material is bound or retained on the column for the neutral pH run than for the low pH run, which, we speculate, is likely due to the larger proteins complex formed at neutral pH conditions.

### Nanoparticle Filter Performance

To understand the complex filtration performance at small scale under elution conditions, 500 μL volume, 300 kDa MWCO PES centrifugal filters (VS0152; Satorius Stedim, PA) were used.

First, two mixtures of antibody and nanoparticle 384 in 50 mM Tris pH 7.4 and 7.8 respectively were combined to a concentration of 0.86 mg/ml and 0.40 mg/ml for antibody and nanoparticle respectively after adding 5M NaCl (in water) to a final concentration of 150 mM. The mixture was either further diluted to pH 3.5 with 100 mM Glycine, pH 3.0 or 50 mM Tris pH 7.4, 150 mM NaCl to a final concentration of 0.20 and 0.43 for nanoparticle 384 and mAb respectively at pH 7.4 or pH 3.5. Each of the two pH mixtures was complemented by a set of two controls containing either the mAb or nanoparticle alone at equivalent protein, NaCl concentration, and pH as in the mixture but replacing the absent component with buffer. In total, six spin filters were first equilibrated (500 μL, 1 spin, 2000 g, 1 minute) in buffer matching the subsequent load and then samples were loaded and spun at 2000 g for 15 s at ambient temperature (RT).

The volume of load, filtrate, and retentate were recorded and absorbance at 280 nm (A280) was measured on a Nanodrop 2000 spectrophotometer (Thermo-Fisher, Waltham MA) blanked with corresponding sample buffer. Samples of 0.5 mL were titrated to neutral pH units using by adding 2% v/v of a 0.5M Tris base solution whose volumetric addition ratio was applied to lower volume load, filtrate and retentate samples as needed and SDS-PAGE was carried out as below.

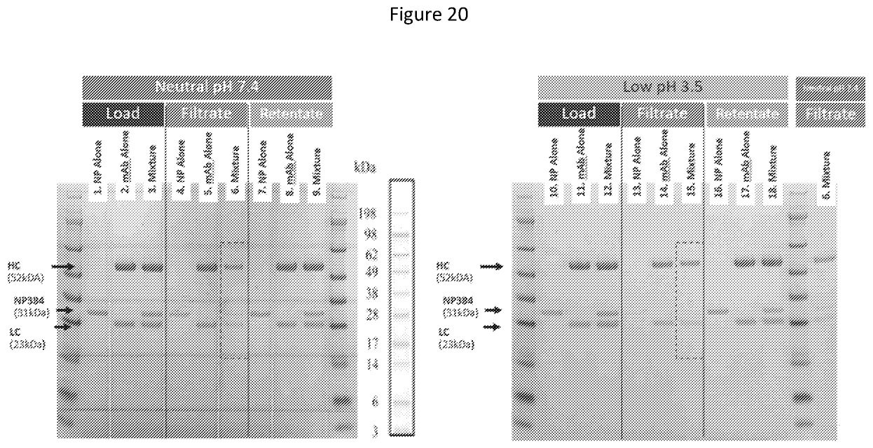

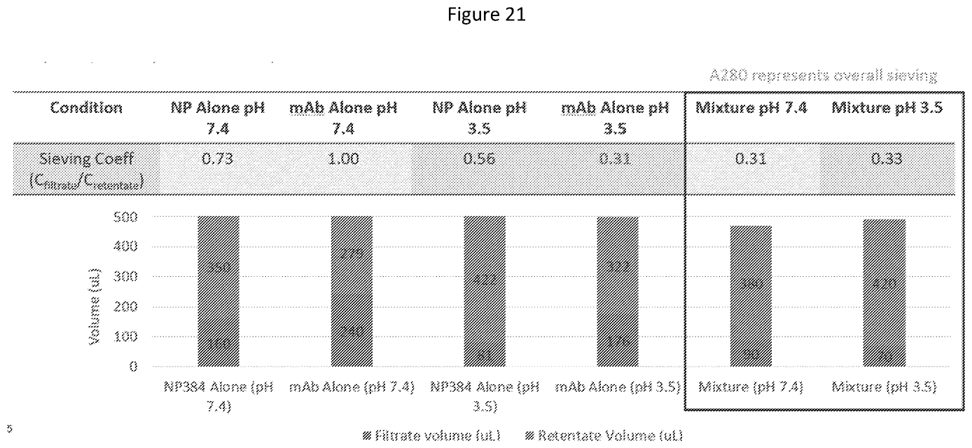

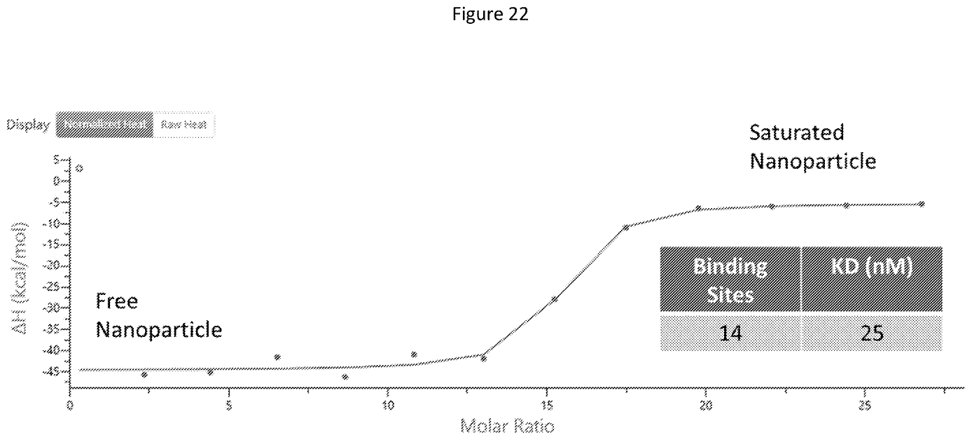

FIG. 20 shows the SDS-PAGE gels for mixtures separated at neutral and low pH. At neutral pH, when the nanoparticle is alone or in the complex it appears both in the filtrate and retentate with less nanoparticle passing the filter when in the complex. The mAb is equally distributed across the filter when alone at neutral pH but is more highly retained when in the complex. At pH 3.5, under elution conditions, a separation is demonstrated where only the mAb passes through the filter when complexed while the nanoparticle is only found in the retentate.

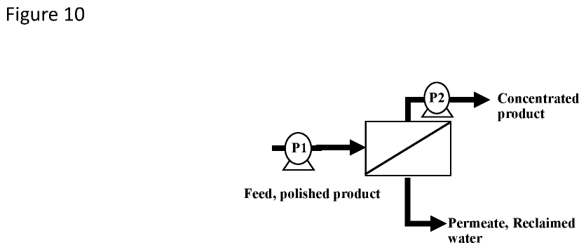

Only small volumes of the load pass through the filter to avoid gel fouling and generate a realistic scale down model of what might occur during TFF (FIG. 10). Negligible volume loss was observed. Sieving for both mAb and nanoparticle decreased with pH while comparable sieving is observed for the mAb complex at each pH.

### Analytical Methods

An Agilent (SantaClara, CA) 1260 Infinity II Bio-inert LC system equipped with an TSKgel SuperSW mAb HR; 4 μm; 7.8 mm×30 cm (Tosoh, King of Prussia, PA) was used to determine purity. Sample was injected and eluted isocratically at 0.5 mL/min for 30 minutes while recording absorbance at 280 nm. An arginine rich mobile phase was used (100 mM Sodium Phosphate Dibasic Anhydrous, 100 mM Sodium Sulfate, 1M Arginine, pH 6.8) and a guard column was not included to avoid material loss.

### SDS PAGE

Sample buffer is prepared by addition Novex™ 2× TrisGlycine SDS (LC2676, Thermo-Fisher) with 5% bME and added at 1:1 volume ratio to the sample, heated to 95° C. for 2 minutes, and cooled to RT. All samples and a SeeBlue™ Plus 2, 3 kDa-198 kDa ladder (LC5925; Thermo-Fisher; not heated/cooled) underwent electrophoresis on a 4-12% bis-tris PAGE with 1×MES running buffer at 200V for 35 min or 150V for 90 min, then was stained with SimplyBlue™ Safestain (LC6060; Thermo-Fisher), and imaged. Low pH samples have an additional 2.5% v/v addition of neat beta-mercapto ethanol after cooling and before loading.

### Example 3: Materials & Methods for TFF and C3ANDO

### Isothermal Calorimetry (ITC)

The titrant material was 28 mg/mL monoclonal antibody (mAb), purified through a bind-elute protein A and flow through anion exchange chromatography to remove impurities. The ITC cell material was 0.5-1.0 g/L nanoparticle and was produced through the methods described in the section. Both resulting materials were buffer exchanged by dialysis using Slide-A-Lyzer™ Dialysis Cassettes, 3.5K MWCO (Thermo Scientific, USA) to a final formulation of 50 mM Tris, pH 7.8, 150 mM NaCl (Tris Saline). This material is degassed using a vacuum degasser. The mAb and nanoparticle concentrations were determined using Solo-VPE (Repligen, USA). A MicroCal PEAQ-ITC Automated (Malvern Panalytical, USA) was used to titrate the ITC cell material (nanoparticle) with a titrant (mab) while measuring the energy released or absorbed by the cell throughout the titration in comparison to a reference cell filled with water for injection (WFI). The titrations were conducted with 12 injections of 3 μL each of titrant, at 2 μL/s, and mixed at 1000 RPM into the ITC sample cell of 200 μL of ITC cell material at 20° C. Control titrations were run with tris saline, nanoparticle, and tris saline as ITC cell materials and mAb, tris saline, and tris saline as titrant material, respectively. The energy released or absorbed by the cell throughout the titration was converted into thermodynamic properties (KD, Enthalpy, Entropy, Free Energy) and molar ratios using the PEAQ-ITC Automated software after inputting concentrations of titrant and ITC cell materials. The three control titrations that were conducted were also uploaded to the software to minimize energy not directly associated with the binding of the nanoparticle with mAb.

### Conditioned Media (CM)-Dewatering

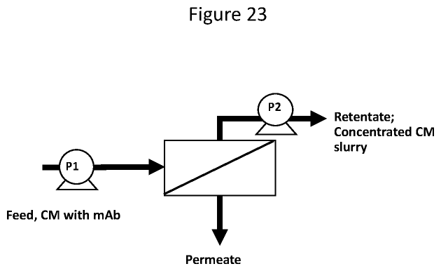

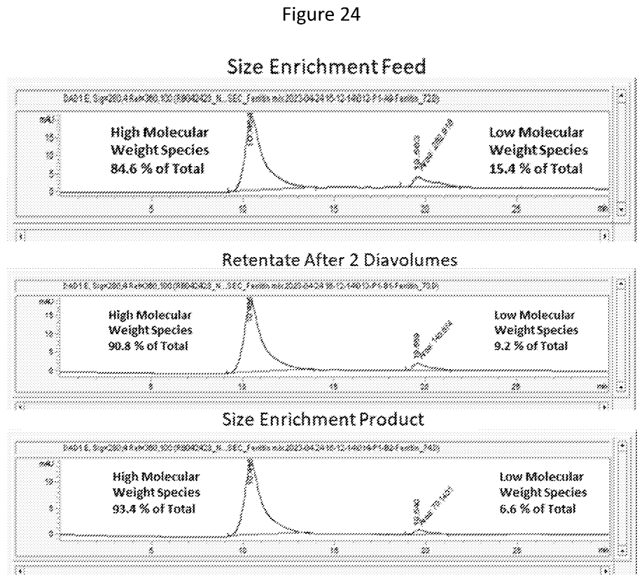

Dewatering conditioned media from perfusion cell culture can significantly reduce the membrane area and water requirements for the subsequent product capture and polishing steps. The dewatering was demonstrated using single-pass TFF operation evaluated in different membrane units/geometries with MWCO of 30-50 kDa. mAb conditioned media (CM) (obtained from perfusion cell culture) containing mAb or mAb fortified CM (mimicking higher titers) was filtered through a 0.22 μm PES filter. The mAb-fortified conditioned media was prepared by spiking purified mAb in mAb-lean CM. As shown in FIG. 23, a continuous single-pass TFF system comprises either a cassette-type membrane or hollow fiber modules and peristaltic pumps.

The feed and retentate flow rates were controlled using peristaltic pumps. This arrangement allows for the regulation of permeate flux and volumetric concentration factor (VCF =Q_{feed}/Qᵣₑₜₑₙₜₐₜₑ). The membrane unit was pre-equilibrated with phosphate-buffered saline, 10 mM sodium phosphate buffer, 150 mM NaCl pH 7.4 by flushing the membranes at a feed flux of 40 LMH for 20 times the hold-up volume of the membrane units. In a typical study, 0.22 μm filtered CM was applied to the TFF module at a feed flux of 10 LMH using the feed pump P1. The flow rates between pumps P1 and P2 are orchestrated to attain the desired permeate flux and VCF along the membrane module, where P1>P2. The choice of VCF is dependent on the mAb concentration in load material and intended dewatering in a single-pass mode. Several excursions of feed flux and mAb concentration were evaluated with respect to process performance measured by transmembrane pressure for membranes ranging from 30-50 kDa MWCO. The concentrated CM was continuously collected at the retentate, while the mAb-depleted CM was collected at the permeate. The mAb concentrations were determined using the analytical protein A column. mAb samples were loaded onto a 0.1 mL POROS™ Prepacked Protein A affinity column (ThermoFisher, Waltham, MA) packed to a bed height of 3.0 cm connected to an Agilent Series Gradient 1200 (Agilent) HPLC system. For all runs, the method had a set linear velocity of 6000 cm/h with a binding buffer of 1×PBS, pH 7.2, and an eluate buffer of 1×PBS, pH 7.2+0.1% H3PO4.

TABLE 6
Summary of flux and feed concentration excursions

| | Concentration | | | HCP in | HCP in |
|---|---|---|---|---|---|
| Flux | of mAb in CM | | HCP in Feed | Retentate | permeate |
| (LMH) | (g/L) | VCF | (ng/mL) | (ng/mL) | (ng/mL) |
| 10 | 1 | 10 | 6.71 × 10⁵ | 4.95 × 10⁴ | 8.58 × 10⁴ |
| 10 | 5 | 10 | 6.11 × 10⁵ | 5.16 × 10⁵ | 1.16 × 10⁵ |
| 10 | 10 | 10 | 6.44 × 10⁵ | 5.65 × 10⁵ | 6.16 10⁵ |

### Nanoparticle Size Enrichment

A feed containing ˜0.5 mg/mL purified nanoparticle (nanoparticle purification methods described earlier) was used in an Ultrafiltration/Diafiltration (UF/DF) step to buffer exchange and remove small-sized (<300 kDa) impurities, specifically monomers and partially formed nanoparticles. The UF/DF was conducted with a 300 kDa PES Pellicon® 2 (Millipore, USA) membrane with a 0.11 m2. Before start of the UF/DF, the membrane storage solution was flushed with 0.25 M NaOH, followed by water (WFI) flush, and then equilibrated with 50 mM Tris, pH 7.8, 150 mM NaCl (Tris Saline). The enrichment was performed at a feed flux of 260 LMH, with ultrafiltration to a concentration of ˜2 mg/mL, followed by 6 DV of diafiltration of tris saline buffer, and a tris saline membrane chase to final enriched nanoparticle at 1.0 mg/mL. Nanoparticle concentrations were determined using Solo-VPE (Repligen, USA) with an extinction coefficient of 0.77 M-1 cm-1. Used membranes were cleaned with 0.25 M NaOH and stored in 0.1 M NaOH for later use.

TABLE 7
Summary of nanoparticle size enrichment

| | Concentration | Yield | Size Purity by |
|---|---|---|---|
| | (g/L) | (%) | SEC (%) |
| Enriched nanoparticle | 1.0 | 71 | 94% |

### Continuous Counter-Current TFF-Capture, HCP Removal, and Elution

Partitioning of a solute during membrane filtration is expressed as the sieving coefficient(S). S is a measure of a solute's ability to pass through a membrane and is calculated by the following equation where Cp is the concentration of solute in permeate, and Cf is the concentration of solute in the retentate.

$$S = \frac{{C}_{p}}{{C}_{f}}$$

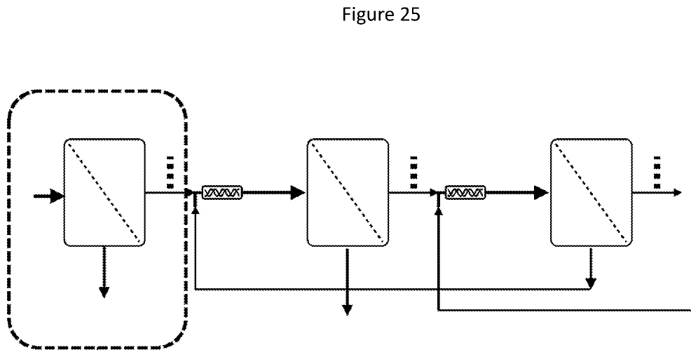

A sieving coefficient of 1 indicates unrestricted transport of the solute through the membrane, while a sieving coefficient of 0 indicates complete retention of the solute by the membrane. Experimental S determination allows the prediction of the ability to separate, recover, or eliminate molecules of interest in the retentate or permeate using a reasonable number of stages in a multi-stage single-pass TFF (SPTFF) setup, as shown in FIG. 25.

### Sieving Coefficient for Pure mAb

Sieving coefficients for pure mAb in tris saline buffer were determined at for various MWCO membranes using an experimental setup shown in FIG. 23 and are show in Table 8.

TABLE 8
Sieving coefficient, S for pure mAb

| | mAb | | | |
|---|---|---|---|---|
| Flux, | concentration, | | Membrane | Sieving |
| LMH | g/L | VCF | MWCO | Coe., S |
| 10 | 5 | 10 | 300 | 1 |
| 10 | 5 | 10 | 500 | 1 |
| 10 | 5 | 10 | 750 | 1 |
| 10 | 5 | 10 | 1000 | 1 |

### Sieving Coefficient for Pure Nanoparticle

Sieving coefficients for pure nanoparticle at pH 7.4 and 3.5 were determined at for 300 kDa MWCO membrane using an experimental setup shown in FIG. 23 and are shown in Table 9.

TABLE 9
Sieving coefficient, S for pure nanoparticle

| Flux, | Nanoparticle, | | | Sieving |
|---|---|---|---|---|
| LMH | g/L | VCF | pH | Coe., S |
| 10 | 0.5 | 10 | 7.8 | 0 |
| 10 | 0.5 | 10 | 3.5 | 0.3 |

### Single-Pass TFF-Binding And Mab-Nanoparticle Complex Retention

In a typical binding step, polished mAb at a concentration of ˜1 mg/mL was extemporaneously mixed equal volume of nanoparticle at 0.5 g/L to attain a (mAb: nanoparticle) molar ratio ˜10. Before mixing, mAb and nanoparticles were exchanged with tris saline buffer at pH 7.4 to avoid any pH/salinity drifts during mixing. The resulting mAb-nanoparticle mixture was then used as feed for a one-stage SPTFF setup with a 300 kDa MWCO membrane module, as shown in FIG. 23. The retentate flow rate was adjusted to attain a VCF of 3 throughout the operation while monitoring the cartridge's TMP and exit flow rates. The free or unbound mAb in the permeate was quantified using ProA bindable HPLC (Table 10).

TABLE 10
Summary of nanoparticle complex retention on TFF

| Flux, | | | % mAb, | % mAb, | TMP, |
|---|---|---|---|---|---|
| LMH | VCF | MWCO | permeate | retentate | psi |
| 10 | 3 | 300 | 15 | 85 | 9 |

As shown in Table 10, 15% of mAb was detected in the permeate, while the remainder of the mAb was retained on the retentate site on a 300 kDa membrane. This observation aligns with the mAb and nanoparticle sieving coefficient results discussed earlier. At pH 7.4, pure mAb shows a S=1, indicating unrestricted partitioning across the membrane, while pure nanoparticle transport is completely restricted, i.e., S=0. Upon binding to the nanoparticle, the resultant affinity complex (>900 kDa) leads to the retention of 85% of the mAb on the retentate side of the membrane while allowing free mAb to be partitioned into permeate.

### Single-Pass TFF HCP Removal

HCP removal is another aspect of any mAb capture process. After successfully demonstrating the formation and retention of the mAb-nanoparticle complex in a pure buffer system, the study was repeated with conditioned media (CM). Typically, perfusion CM with ˜1 mg/mL mAb titer was extemporaneously mixed with equal volume of 0.5 g/L nanoparticle in tris saline pH 7.8 to attain a (mAb: nanoparticle) molar ratio ˜10. The one-stage SPTFF setup with a 300 and 1000 kDa MWCO membrane module was pre-charged with CM by flushing membranes at 40 LMH feed flow rate for 20× hold-up volume of the system. Following pre-charging, the mAb-nanoparticle mixture was used as feed for the SPTFF setup as shown in FIG. 23. The retentate flow rate was adjusted to attain a VCF of 3 throughout the operation while monitoring the cartridge's TMP and exit flow rates. The free or unbound mAb in the permeate was quantified using ProA bindable HPLC (Table 11).

TABLE 11
HCP removal in TFF for 300 and 1000 kDa MWCO membranes

| | | | | | HCP | HCP | HCP | |
|---|---|---|---|---|---|---|---|---|
| Flux, | | MWCO, | % mAb, | % mAb, | Feed, | Permeate, | Retentate, | TMP, |
| LMH | VCF | kDa | permeate | retentate | ng/mL | ng/mL | ng/mL | psi |
| 10 | 3 | 300 | 11 | 89 | 193,000 | 148,000 | 320,000 | 5 |
| 10 | 3 | 1000 | 5 | 95 | 193,000 | 183,000 | 286,000 | 1 |

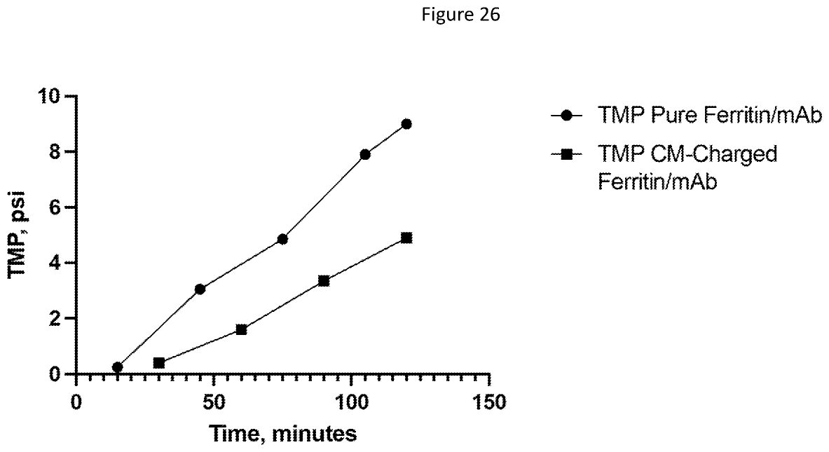

Similar to earlier results, 11% of mAb was detected in the permeate, while the remainder of the mAb was retained on the retentate side on a 300 kDa membrane. The extent of HCP removal observed can be further improved by multistage SPTFF setup with appropriate wash buffers. For 300 kDa membrane, a distinctly lower operating TMP of 5 psi was observed (FIG. 26) when compared to 9 psi for mAb-nanoparticle in pure buffer system.

### Single-Pass TFF-Elution

To assess the retrieval of mAb during elution, we employed the setup shown in FIG. 23. The procedure comprises gradually adjusting the pH of the solution containing the mAb-nanoparticle complex to 3.5 from 7.8 using 2M Glycine HCl pH 3.0 buffer. The acidified solution served as feed for a one-stage single-pass TFF setup, which employed a 300 kDa MWCO membrane module. Throughout the operation, the retentate flow rate was adjusted to achieve a VCF of 3 while closely monitoring the cartridge's TMP and exit flow rates. Samples collected at retentate and permeate were reduced and denatured at 70° C. for 5 min, then loaded on an SDS-PAGE gel for electrophoresis with 1× NuPAGE™ MES SDS buffer supplemented with 1 mM Sodium metabisulfite. Following electrophoresis, the gel was transferred to a fixing solution, and Coomassie blue staining was applied, followed by destaining with deionized water. The mAb recovery in the permeate with respect to feed was calculated using image densitometry using ImageJ software.

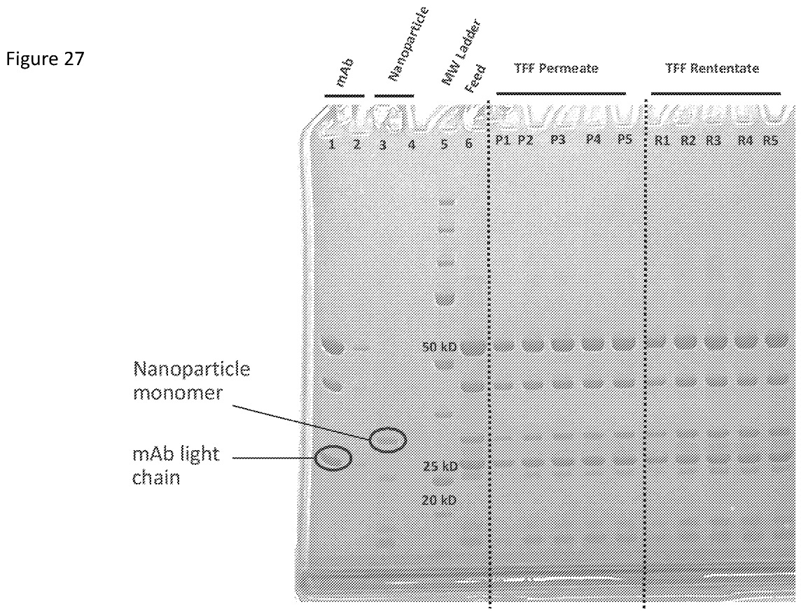

The analysis of the SDS-PAGE gel image (FIG. 27) reveals that the mAb recovery in permeate was 64%, which is comparable to the maximum theoretical recovery (66%) for a molecule with S=1 on a 300 kDa membrane at VCF of 3 for a one-stage setup. However, a multistage setup has the potential to increase the mAb recovery up to 90%. On the other hand, in the case of nanoparticles, approximately 50% of the nanoparticle was found to be partitioned into permeate, which is higher than the 20% partitioning observed with pure nanoparticles at pH 3.5.

TABLE 12
Results of mAb elution in TFF from image densitometry

| Flux, | | MWCO, | % mAb, | % mAb, | % Nanoparticle, | % Nanoparticle, |
|---|---|---|---|---|---|---|
| LMH | VCF | kDa | permeate | retentate | permeate | permeate |
| 10 | 3 | 300 | 64 | 34 | 47 | 32 |

### Example 4: Continuous Counter-Current Affinity Nanoparticle Dialysis Operation (C3ANDo)

### Diffusion Coefficient (De) for mAb

Convection and diffusion are two mechanisms that drive molecules' movement across a semipermeable membrane. Unlike convection, diffusion is driven by differences in concentration gradient across the membrane. A membrane's effectiveness in facilitating diffusion depends on the membrane's properties and the proteins' size and shape. Therefore, the measurement of real-world mAb diffusivity or diffusion coefficient allows the estimation of mass transfer in a diffusion-dominated system. The following text depicts a typical C3 ANDo setup using a hollow fiber membrane that is operated in a counter-current manner. The C3ANDo setup discussed here was used to evaluate diffusivities for mAb and other model proteins, as shown in Table 13 below, with multiple membranes followed by a demonstration of mAb capture and elution.

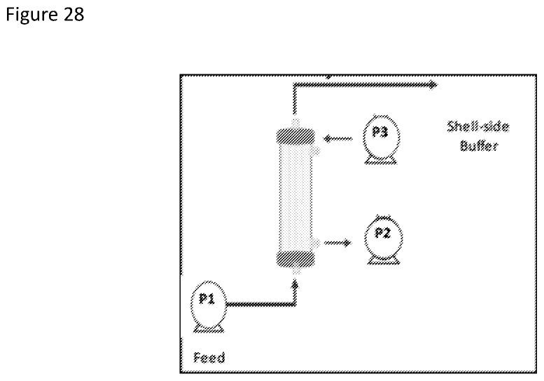

A hollow fiber module was mounted in a vertical orientation where the feed was introduced from the bottom port on the lumen side through pump P1 (FIG. 28). The shell-side port proximal to the feed port (shell-outlet) was attached to pump P2. The distal shell-side (shell-inlet) port was attached to pump P3. The shell-side buffer was introduced into the module using pump P3, while pump P2 modulated the flow rate at the shell outlet. The depleted stream was collected at the lumen outlet. The peristaltic pumps, P1, P2, and P3 were equipped with appropriate pump heads and tubing. Pressures were monitored using pressure sensors placed immediately before and after the inlet/outlet ports. The shell and lumen compartments of the hollow fiber module were flushed with buffer prior to the experiment. The relationship between the feed and shell side flow rate was dictated using α, where:

$$α = \frac{S h ell - side buffer flow rate}{Feed flow rate}$$

In a typical experimental setup with feed flow (P1) of 0.05 ml/min (Flux 0.12 LMH), both shell-side pumps were adjusted to 0.112 mL/min for an a value of 2.25. For some experiments, the values ranging from 0.2 to 2.25 were evaluated. The setup was operated in single-pass or feed recirculation mode. Small samples were collected periodically from the lumen outlet and shell-outlet for offline analysis.

TABLE 13
Experimentally measured diffusion coefficient (De) for proteins

| Membrane MWCO; | | |
|---|---|---|
| wall thickness | Protein/Conditions | De (m²/s) |
| ~500 kDa; 40 μm | mAb | 1.0 to 1.4 × 10⁻¹¹ |
| ~500 kDa; 40 μm | Myoglobin | 6.6 × 10⁻¹¹ |
| ~500 kDa; 40 μm | mAb in conditioned media | 0.5 × 10⁻¹¹ |
| 300 kDa; 200 μm | Myoglobin | 2.5 × 10⁻¹¹ |
| 300 kDa; 200 μm | Ovalbumin | 1.6 × 10⁻¹¹ |

### C3ANDo Capture

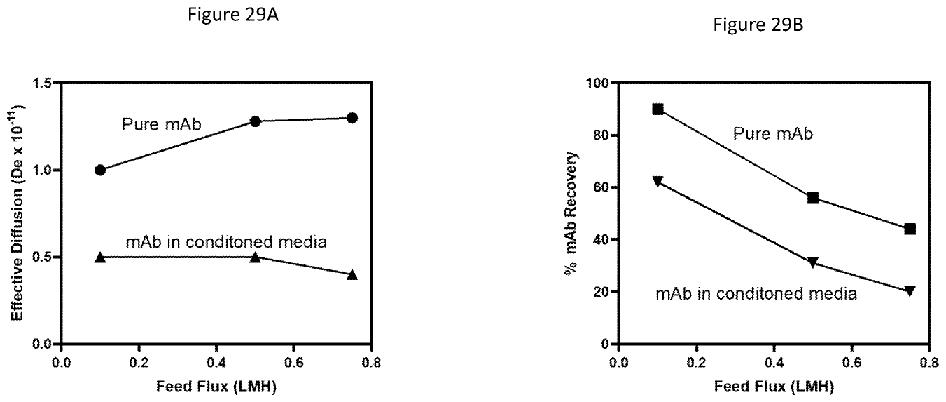

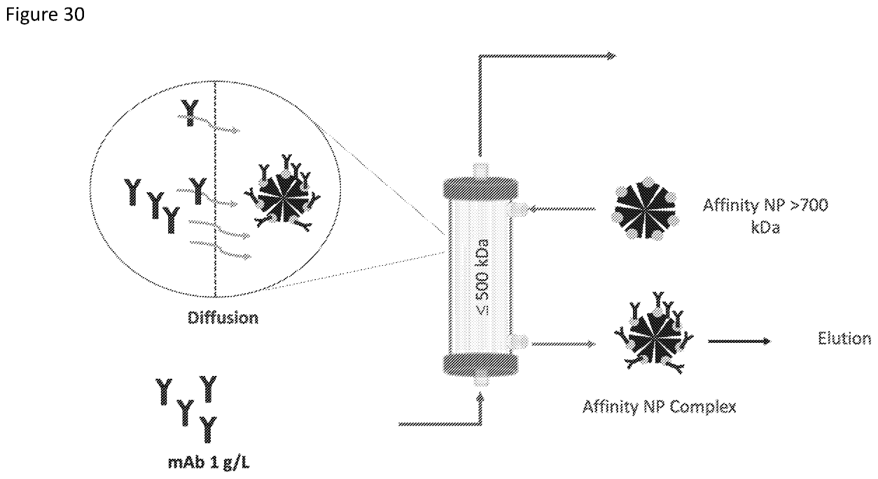

In a typical mAb capture study, 1 g/L polished mAb in tris saline buffer pH 7.8 was used as the feed, while tris saline buffer pH 7.8 was simultaneously introduced into the shell-side inlet. The feed and shell-side flow rates were kept at 0.05 ml/min and 0.01 mL/min resulting in a of 0.2. To evaluate the effect of affinity nanoparticle on mAb recovery, a similar experiment was conducted wherein the shell-side inlet was supplied with nanoparticle in tris saline buffer pH 7.8, as shown in FIGS. 29A and 29B. The mAb recovery on the shell side was calculated by quantifying the mAb in the lumen outlet using Solo-VPE (Repligen, USA).

As shown in Table 14 below, the presence of nanoparticle on the shell-side increases the mAb recovery compared to the buffer alone. The formed mAb-nanoparticle complex was collected at the shell-outlet and was then utilized for evaluating mAb recovery during the C3ANDo elution step.

TABLE 14
Effect of shell-side nanoparticle on mAb recovery during capture

| | mAb Recovery | % Increase |
|---|---|---|
| Shell-side compartment | on shell-side | in recovery |
| Tris Saline pH 7.8 | 25% | N/A |
| Nanoparticle + Tris saline pH 7.8 | 40% | 60% |

### C3ANDo Elution

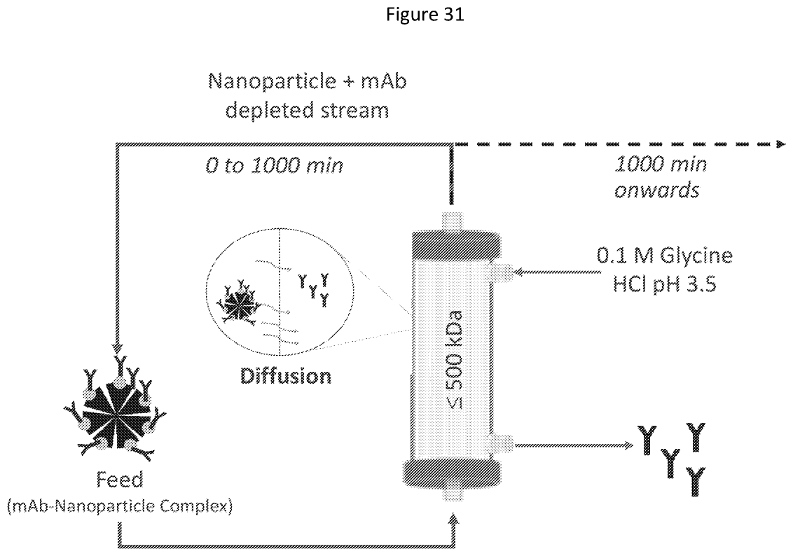

The feasibility of the diffusion-driven elution approach was evaluated using the scheme shown in FIG. 31. To demonstrate C3ANDo elution, the mAb-nanoparticle complex (pH 7.8) obtained from the capture step was dialyzed against 0.1 M glycine HCl pH 3.5 buffer. As the feed solution travels along the membrane, the solution pH changes from 7.8 to 3.5 due to buffer exchange resulting in the dissociation of the mAb (150 kDa) from the nanoparticle (750 kDa). Owing to its relatively small size, the dissociated mAb preferentially diffuses across the membrane to the shell-side compartment.

The lumen-outlet stream was recirculated through the membrane. During the feed re-circulation, the eluted mAb was collected at the shell-side outlet. After 1000 minutes of runtime, the feed recirculation was stopped, the operation was continued in single-pass mode, and the lumen-outlet stream was collected. Wherever applicable, the samples were neutralized to pH 7.4 using 0.5 M Tris base. These samples were reduced and denatured at 70° C. for 5 min, then loaded on an SDS-PAGE gel for electrophoresis with 1× NuPAGE™ MES SDS buffer supplemented with 1 mM Sodium metabisulfite. Following electrophoresis, the gel was transferred to a fixing solution, and Coomassie blue staining was applied, followed by destaining with deionized water. The mAb recovery in the permeate with respect to feed was estimated using image densitometry using ImageJ software.

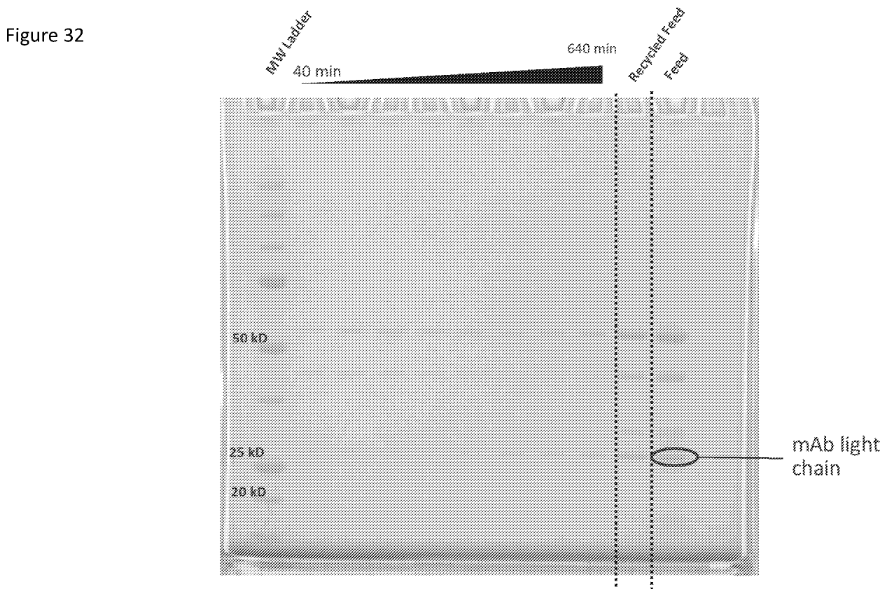

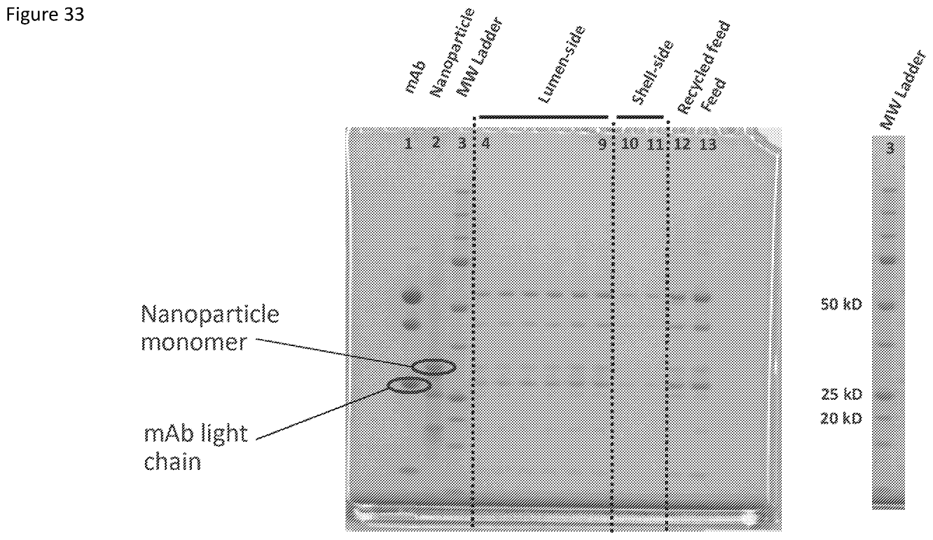

As described above, the C3ANDO elution study was split into two operational modes, a recirculation mode (0-1000 min; FIG. 32) and single-pass mode (1000-1600 min; FIG. 33). From FIG. 32, it can be observed that an increasing amount of mAb diffuses in the shell compartment of the hollow fiber (40-640 min) without any detectable nanoparticle when compared to feed. The presence of low pH on the shell side causes mAb to dissociate/elute from the nanoparticle, followed by diffusion across the membrane to the shell-side compartment. Compared to the feed, a noticeable reduction in mAb concentration can be seen in the recirculated feed sample (FIG. 32). In FIG. 33, wells 4-9 correspond to lumen-outlet samples collected at time points 1040, 1120, 1240, 1320, 1440, and 1520 min after switching to the single-pass mode at 1000 min. These samples show relative enrichment of the nanoparticle as the system reaches a steady state. The eluted mAb from the re-circulation phase, 600 and 640 min time points, was loaded alongside the single-pass samples in wells 10 and 11 (FIG. 33), respectively, for comparison.

Preferred aspects of the disclosure are described herein. Variations of those preferred aspects may become apparent to those of ordinary skill in the art upon reading the foregoing description. Accordingly, this disclosure includes any combination of the above-described elements in all possible variations thereof is encompassed by the invention unless otherwise indicated herein or otherwise clearly contradicted by context.

1. A method for purifying a protein product of interest using counter-current flow filtration, comprising:
(a) contacting a first solution comprising the protein product of interest and impurities with a binding molecule to form a complex, wherein the complex comprises the protein product of interest bound to the binding molecule and wherein the binding molecule comprises Protein A, Protein G, cation exchange resin, or anion exchange resin;
(b) contacting a first flow solution comprising the complex with a first side of a semi-permeable membrane, wherein the complex has a molecular weight that exceeds the molecular weight cutoff of the semi-permeable membrane such that the complex is retained on the first side of the membrane;
(c) passing the impurities through the semi-permeable membrane, wherein the impurities have a molecular weight below the molecular weight cutoff of the semi-permeable membrane and are retained on the second side of the semi-permeable membrane in a second flow solution that is counter-current to the first flow solution; and
(d) dissociating the complex to form free protein product of interest and free binding molecule; wherein the protein product of interest comprises an antibody, an antigen binding fragment, a fusion protein, a naturally occurring protein, a chimeric protein, or any combination thereof and wherein steps (a) through (c) are repeated.

2. (canceled)

3. A method for purifying a protein product of interest using counter-current flow filtration, comprising:
(a) contacting a first flow solution comprising the protein product of interest and impurities with a first side of a semi-permeable membrane, wherein the protein product of interest passes through the semi-permeable membrane to form a complex with a binding molecule on a second side of a semi-permeable membrane, wherein the complex has a molecular weight that exceeds the molecular weight cutoff of the semi-permeable membrane such that the complex is retained on the second side of the semi-permeable membrane and wherein the binding molecule comprises Protein A, Protein G, cation exchange resin, or anion exchange resin;
(b) optionally retaining the impurities on the first side of a semi-permeable membrane or wherein the impurities flow through the semi-permeable membrane, wherein the impurities have a molecular weight below the molecular weight cutoff of the semi-permeable membrane and are either retained on the first side of the semi-permeable membrane in the first flow solution that is counter-current to the second flow solution, or pass through the semi-permeable membrane to the second side of the semi-permeable membrane; wherein the second flow solution flow rate is lower than first flow solution flow rate, and
(c) dissociating the complex to form free protein product of interest and free binding molecule;
wherein unbound binding molecules diffuse through the semi-permeable membrane into the second flow solution, and wherein steps (a) through (c) are repeated.

4. (canceled)

5. The method of claim 1, further comprising:
(e) regenerating the binding molecule, wherein the regenerated binding molecule is capable of forming a complex upon contact with the protein product of interest in the first flow solution or the second flow solution, wherein the regenerated binding molecule is again passed through the second side of the semi-permeable membrane in the second flow solution; and wherein unbound binding molecules diffuse through the semi-permeable membrane into the second flow solution.

6. The method of claim 1, wherein the second flow solution comprises a second binding molecule that can bind to impurities in the first solution and/or second flow solution.

7. (canceled)

8. (canceled)

9. (canceled)

10. (canceled)

11. (canceled)

12. The method of claim 1, wherein the second flow solution comprises a positively charged polymer, wherein the positively charged polymer binds low molecular weight species that have diffused through the semi-permeable membrane.

13. (canceled)

14. (canceled)

15. (canceled)

16. (canceled)

17. (canceled)

18. (canceled)

19. (canceled)

20. The method of claim 1, wherein the binding molecule comprises an assembled ferritin nanoparticle comprising 24 fusion protein monomers;
wherein each fusion protein monomer comprises i) a self-assembling nanoparticle monomer; ii) a linker; and iii) an immunoglobulin binding domain; wherein the immunoglobulin binding domain is a protein A Z-domain comprising an amino acid sequence at least 90%, 95%, 98% or 100% identical to one of SEQ ID NOs: 4-7.

21. (canceled)

22. The method of claim 20, wherein the fusion protein monomer comprises
i. an amino acid sequence at least 90%, 95%, 98% or 100% identical to one of SEQ ID NOs: 1-3;
ii. an amino acid sequence at least 90%, 95%, 98% or 100% identical to one of SEQ ID NOs: 28-36′
iii. an amino acid sequence at least 90%, 95%, 98% or 100% identical to SEQ ID NO: 29, SEQ ID NO: 30 or SEQ ID NO: 32; or
iv. an amino acid sequence encoded by a nucleic acid comprising SEQ ID NO 52.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The method of claim 1, wherein the permeate from step (b) and/or step (c) is recycled into the first flow solution and/or the second and/or an upstream flow solution.

40. (canceled)

41. (canceled)

42. The method of claim 1, wherein the first flow solution and/or second flow solution has a flow rate of about 30 to about 60 mL/minute.

43. The method of claim 42, wherein the first flow solution and second flow solution have the same flow rate.

44. The method of claim 42, wherein the first flow solution and second flow solution have different flow rates.

45. The method of claim 1, wherein the first flow solution and/or second solution are pulsed.

46. (canceled)

47. The method of claim 45, wherein the pulse volume is less than the volume of a pore of the semi-permeable membrane.

48. (canceled)

49. (canceled)

50. (canceled)

51. (canceled)

52. (canceled)

53. (canceled)

54. (canceled)

55. (canceled)

56. (canceled)

57. The method of claim 1, wherein the free protein product of interest is concentrated to about 50 g/L to about I 00 g/L.

58. (canceled)

59. The method of any claim 1, wherein steps (a) through (c) are performed a second time and wherein the second flow solution of the second time is added to the first flow solution of the first time, and wherein the MWCO of the filter used the second time has a MWCO the same size or larger than steps (a)-(c) performed the first time.

60. The method of claim 3, wherein steps (a) through (c) are performed a second time and wherein the first flow solution of the second time is added to the first flow solution of the first time, and wherein the MWCO of the filter used the second time has a MWCO the same size or larger than steps (a)-(c) performed the first time.

61. The method of claim 59, wherein steps (a) through (c) are performed a third time and wherein the second flow solution of the third time is added to the first flow solution of the second time, and wherein the MWCO of the filter used the third time has a MWCO the same size or larger than steps (a)-(c) performed the first time or the second time.

62. The method of claim 60, wherein steps (a) through (c) are performed a third time and wherein the first flow solution of the third time is added to the first flow solution of the second time, and wherein the MWCO of the filter used the third time has a MWCO the same size or larger than steps (a)-(c) performed the first time or the second time.

63. (canceled)

64. The method of claim 59, wherein the first flow solution or the second flow solution of the third time flows into the first flow solution of the second time, and/or wherein the first flow solution or the second flow solution of the second time flows into the first flow solution of the first time.

65. A method of using a solution effluent from the filtrate or dialysate or the retentate of a continuous downstream step as a wash for an upstream step, wherein
i. when the effluent or dialysate is the wash, the upstream step has a filter with a MWCO the same size or larger than the downstream step that generated the effluent; and
ii. when the retentate is the wash, the upstream step has a filter with a MWCO the same size or smaller than the downstream step that generated the effluent, and the filtrate is directed downstream.

66. (canceled)


SEQUENCE LISTING (52 sequences)

SEQ ID NO: 1 - protein, 173 aa, Helicobacter pylori
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 2 - protein, 174 aa, Pyrococcus furiosus
MLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 3 - protein, 108 aa, synthetic construct
MATRTPKLVKHTLLTRFKDEITREQIDNYINDYTNLLDLIPSMKSFNWGTDLGMESAELNRGYTHAFESTFESKSGLQEYLDSAALAAFAEGFLPTLSQRLVIDYFLY

SEQ ID NO: 4 - protein, 58 aa, Staphylococcus aureus [sequence omitted]

SEQ ID NO: 5 - protein, 58 aa, synthetic construct
VDNKFNKEQHHAFYEILHLPNLNEEQRNAFIQSLKDDPSQSANLLAEAKKLNDAQAPK

SEQ ID NO: 6 - protein, 58 aa, synthetic construct
VDNKFNKEQHHAFYEILSLPNLNEEQRAAFIQSLKDDPSQSANLLAEAKKLNDAQAPK

SEQ ID NO: 7 - protein, 69 aa, synthetic construct
VDNKLNKEQQNAFYEILHLPNLNEEQRKAFIQSLIDGGGDTNGNGYLDAEESANLLAEAKKLNDARAPK

SEQ ID NO: 8 - protein, 74 aa, synthetic construct
AGSGGSGGSGGSPVPSTPPTPSPSTPPTPSPSPVPSTPPTPSPSTPPTPSPSPVPSTPPTPSPSTPPTPSPSAS

SEQ ID NO: 9 - protein, 52 aa, synthetic construct
ASGAPGPGGAGPGGAGPGGAGPGGAGPGGAGPGGAGPGGAGPGGAGAGRSSG

SEQ ID NO: 10 - protein, 71 aa, synthetic construct
ASGASMALEKMALEKMALEKGSMALEKMALEKMALEKMALEKMALEKGSMALEKMALEKMALEKPSGRSSG

SEQ ID NO: 11 - protein, 118 aa, synthetic construct
ASGAPGPGGAGPGGAGPGGAGPGGAGPGGAGPGGAGPGGAGPGGAGAGRNASMALEKMALEKMALEKGSMALEKMALEKMALEKMALEKMALEKGSMALEKMALEKMALEKPSGRSSG

SEQ ID NO: 12 - protein, 4 aa, synthetic construct
PPPP

SEQ ID NO: 13 - protein, 25 aa, synthetic construct
SGSVTSTSKTTATASKTSTSTSSTS

SEQ ID NO: 14 - DNA, 45 bp, synthetic construct
  source 1..45: mol_type = other DNA
gaagctgctgctaaagaagctgctgctaaagaagctgctgctaaa

SEQ ID NO: 15 - protein, 55 aa, synthetic construct
LFNKEQQNAFYEILHLPNLNEEQRNGFIQSLKDDPSQSANLLAEAKKLNDAQAAA

SEQ ID NO: 16 - protein, 30 aa, synthetic construct
LAEAAAKEAAAKEAAAKEAAAKEAAAKAAA

SEQ ID NO: 17 - protein, 25 aa, synthetic construct
SGSVTSTSKTTATASKTSTSTSSTS

SEQ ID NO: 18 - protein, 30 aa, synthetic construct
AVPSGSVTSTSKTTTTASKTSTSTSSTSEF

SEQ ID NO: 19 - protein, 46 aa, synthetic construct
GGLNDIFEAQKIEWHEGKKGKGPQPQPKPQPQPQPQPKPQPKPEPE

SEQ ID NO: 20 - protein, 28 aa, synthetic construct
GPGPGHHHHHHELGGGGSGGGGSGGGGS

SEQ ID NO: 21 - protein, 6 aa, synthetic construct
GSGGGG

SEQ ID NO: 22 - protein, 13 aa, synthetic construct
MHWSAWWIRSNQS

SEQ ID NO: 23 - protein, 19 aa, synthetic construct
GSGGGGGSGGGGAEAAAKA

SEQ ID NO: 24 - protein, 5 aa, synthetic construct
GGGGS

SEQ ID NO: 25 - protein, 10 aa, synthetic construct
GGGGSGGGGS

SEQ ID NO: 26 - protein, 23 aa, synthetic construct
GSEAAAKEAAAKEAAAKGSGGGG

SEQ ID NO: 27 - protein, 6 aa, synthetic construct
GSGGGG

SEQ ID NO: 28 - protein, 260 aa, synthetic construct
MGHHHHHHHHGPLEVLFQGPVDNKFNKEQHHAFYEILHLPNLNEEQRNAFIQSLKDDPSQSANLLAEAKKLNDAQAPKAAAGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 29 - protein, 260 aa, synthetic construct
MGHHHHHHHHGPLEVLFQGPVDNKFNKEQQNAFYEILHLPNLNEEQRNAFIQSLKDDPSQSANLLAEAKKLNDAQAPKAAAGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 30 - protein, 274 aa, synthetic construct
MGHHHHHHHHGPLEVLFQGPVDNKFNKEQQNAFYEILHLPNLNEEQRNAFIQSLKDDPSQSANLLAEAKKLNDAQAPKGSEAAAKEAAAKEAAAKGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 31 - protein, 274 aa, synthetic construct
MGHHHHHHHHGPLEVLFQGPVDNKFNKEQHHAFYEILHLPNLNEEQRNAFIQSLKDDPSQSANLLAEAKKLNDAQAPKGSEAAAKEAAAKEAAAKGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 32 - protein, 271 aa, synthetic construct
MGHHHHHHHHGPLEVLFQGPVDNKLNKEQQNAFYEILHLPNLNEEQRKAFIQSLIDGGGDTNGNGYLDAEESANLLAEAKKLNDARAPKAAAGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 33 - protein, 184 aa, synthetic construct
MGVDNKFNKEQHHAFYEILSLPNLNEEQRAAFIQSLKDDPSQSANLLAEAKKLNDAQAPKAAAMHWSAWWIRSNQSMATSTPKLVKHTLLTRFKDEITREQIDNYINDYTNLLDLIPSMKSFNWGTDLGMESAELNRGYTHAFESTFESKSGLQEYLDSAALAAFAEGFLPTLSQRLVIDYFLY

SEQ ID NO: 34 - protein, 126 aa, synthetic construct
MGAAAMHWSAWWIRSNQSMATSTPKLVKHTLLTRFKDEITREQIDNYINDYTNLLDLIPSMKSFNWGTDLGMESAELNRGYTHAFESTFESKSGLQEYLDSAALAAFAEGFLPTLSQRLVIDYFLY

SEQ ID NO: 35 - protein, 322 aa, synthetic construct
MGHHHHHHHHGPLEVLFQGPVDNKFNKEQHHAFYEILHLPNLNEEQRNAFIQSLKDDPSQSANLLAEAKKLNDAQAPKVDNKFNKEQHHAFYEILHLPNLNEEQRNAFIQSLKDDPSQSANLLAEAKKLNDAQAPKAAAGGGGSGGGGSESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 36 - protein, 307 aa, synthetic construct
MGVDNKFNKEQHHAFYEILSLPNLNEEQRAAFIQSLKDDPSQSANLLAEAKKLNDAQAPKVDNKFNKEQHHAFYEILSLPNLNEEQRAAFIQSLKDDPSQSANLLAEAKKLNDAQAPKAAAGSGGGGGSGGGGMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 37 - DNA, 213 bp, synthetic construct
  source 1..213: mol_type = other DNA
gcgagtggcgcatctatggcgttggaaaaaatggcgcttgaaaagatggccctggaaaagggaagtatggctttggaaaagatggccttggagaaaatggcactggagaagatggccttagagaagatggcattagaaaaaggtagcatggctcttgaaaaaatggcccttgagaaaatggcgctggaaaaaccttcgggtcgctcaagtggc

SEQ ID NO: 38 - DNA, 75 bp, synthetic construct
  source 1..75: mol_type = other DNA
tccggcagcgtgacctcgaccagcaagaccaccgcgactgctagcaagaccagcaccagtacgtcttcaacctcc

SEQ ID NO: 39 - DNA, 90 bp, synthetic construct
  source 1..90: mol_type = other DNA
gcggtgccgagcggcagcgtgaccagcaccagcaaaaccacaaccaccgcgagcaaaaccagcaccagcaccagcagcaccagcgaattt

SEQ ID NO: 40 - DNA, 87 bp, synthetic construct
  source 1..87: mol_type = other DNA
ggcggcctgaacgatatttttgaagcgcagaaaattgaatggcatgaaggcaagaaaggcaaaggcccgcagccgcagccgaaaccc

SEQ ID NO: 41 - DNA, 84 bp, synthetic construct
  source 1..84: mol_type = other DNA
ggtccaggtccgggtcatcaccatcaccaccacgaactgggtggtggtggttctggtggtggcggttctggtggtggtggttct

SEQ ID NO: 42 - DNA, 18 bp, synthetic construct
  source 1..18: mol_type = other DNA
ggatctggtggtggaggg

SEQ ID NO: 43 - DNA, 39 bp, synthetic construct
  source 1..39: mol_type = other DNA
atgcattggagcgcgtggtggattcgcagcaaccagagc

SEQ ID NO: 44 - DNA, 57 bp, synthetic construct
  source 1..57: mol_type = other DNA
ggctctggtggtggagggggatctggtggtggcggggcggaggcggccgcaaaagcg

SEQ ID NO: 45 - DNA, 15 bp, synthetic construct
  source 1..15: mol_type = other DNA
ggtggtggagggtct

SEQ ID NO: 46 - DNA, 30 bp, synthetic construct
  source 1..30: mol_type = other DNA
ggaggcggtggttctgggggcggtggcagc

SEQ ID NO: 47 - DNA, 69 bp, synthetic construct
  source 1..69: mol_type = other DNA
ggcagtgaagcagccgcgaaggaagccgcagcaaaggaggcggcagccaagggatctggtggtggaggg

SEQ ID NO: 48 - DNA, 18 bp, synthetic construct
  source 1..18: mol_type = other DNA
ggatctggcggaggaggg

SEQ ID NO: 49 - protein, 8 aa, synthetic construct
HHHHHHHH

SEQ ID NO: 50 - protein, 10 aa, synthetic construct
GPLEVLFQGP

SEQ ID NO: 51 - protein, 20 aa, synthetic construct
MGHHHHHHHHGPLEVLFQGP

SEQ ID NO: 52 - DNA, 822 bp, synthetic construct
  source 1..822: mol_type = other DNA
atgggccatcaccatcaccaccaccatcatggacccctcgaagtattgttccagggtcctgtggataacaaattcaacaaagaacaacaaaatgctttctatgaaatcttacatttacctaacttaaacgaagaacaacgcaatgcgttcatccaaagcctgaaagatgacccaagccaaagcgctaaccttttagcagaagctaaaaagctgaatgatgctcaagcaccaaaaggcagtgaagcagccgcgaaggaagccgcagcaaaggaggcggcagccaagggatctggtggtggaggggaaagccaagtccgccagcaattttcaaaagatatcgaaaaactgttgaacgagcaagtaaataaagaaatgcagtccagtaacctctatatgtccatgtcctcctggtgctatacgcacagtctggacggcgcgggtctttttctgtttgaccatgccgctgaagagtacgagcacgctaagaaattgatcatctttctgaatgagaacaatgtccctgttcaattgaccagtatttccgctccggaacataagtttgagggattgacgcagatttttcagaaagcctatgagcatgagcaacacatctcagagagcatcaacaacatcgtggaccatgcgatcaagtcaaaagaccacgctactttcaacttccttcaatggtatgtagccgaacaacacgaggaggaggtcctgttcaaagatatcttggataaaattgaactcattggaaatgagaatcatggcctgtacctcgctgaccagtatgttaaagggattgctaagagtcgcaagtca